SEQUENCE LISTING (277 sequences)

SEQ ID NO: 1 - protein, 124 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
GFTFRDYA

SEQ ID NO: 4 - protein, 8 aa, synthetic construct
ISGSGGNT

SEQ ID NO: 5 - protein, 18 aa, synthetic construct
AKDRLSITIRPRYYGLDV

SEQ ID NO: 6 - protein, 11 aa, synthetic construct
QSLLYSIGYNY

SEQ ID NO: 7 - skipped

SEQ ID NO: 8 - protein, 9 aa, synthetic construct
MQALQTPYT

SEQ ID NO: 9 - protein, 452 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 219 aa, synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 207 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 12 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 13 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 14 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 15 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 16 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 17 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 18 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 19 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 20 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 21 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 22 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 23 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 24 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 25 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 26 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 27 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 28 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 29 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 30 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 31 - protein, 109 aa, synthetic construct
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 32 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 35 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 36 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 37 - protein, 115 aa, synthetic construct
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, synthetic construct
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, synthetic construct
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 40 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 41 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 45 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 46 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 47 - protein, 115 aa, synthetic construct
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, synthetic construct
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, synthetic construct
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, synthetic construct
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 51 - protein, 115 aa, synthetic construct
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 52 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 53 - protein, 115 aa, synthetic construct
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 54 - protein, 115 aa, synthetic construct
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, synthetic construct
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, synthetic construct
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 57 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 58 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 59 - protein, 115 aa, synthetic construct
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, synthetic construct
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, synthetic construct
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 63 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 64 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 65 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 66 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 67 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 68 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 69 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 70 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 71 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 72 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 73 - protein, 116 aa, synthetic construct; identical to SEQ ID NO: 69

SEQ ID NO: 74 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 75 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 76 - protein, 111 aa, synthetic construct; identical to SEQ ID NO: 74

SEQ ID NO: 77 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 78 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 79 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 80 - protein, 111 aa, synthetic construct; identical to SEQ ID NO: 74

SEQ ID NO: 81 - protein, 116 aa, synthetic construct; identical to SEQ ID NO: 79

SEQ ID NO: 82 - protein, 111 aa, synthetic construct; identical to SEQ ID NO: 78

SEQ ID NO: 83 - protein, 116 aa, synthetic construct; identical to SEQ ID NO: 69

SEQ ID NO: 84 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 85 - protein, 116 aa, synthetic construct; identical to SEQ ID NO: 75

SEQ ID NO: 86 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 87 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 88 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 89 - protein, 116 aa, synthetic construct; identical to SEQ ID NO: 87

SEQ ID NO: 90 - protein, 111 aa, synthetic construct; identical to SEQ ID NO: 84

SEQ ID NO: 91 - protein, 116 aa, synthetic construct; identical to SEQ ID NO: 75

SEQ ID NO: 92 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 93 - protein, 116 aa, synthetic construct; identical to SEQ ID NO: 69

SEQ ID NO: 94 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 95 - protein, 116 aa, synthetic construct; identical to SEQ ID NO: 69

SEQ ID NO: 96 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 97 - protein, 116 aa, synthetic construct; identical to SEQ ID NO: 69

SEQ ID NO: 98 - protein, 111 aa, synthetic construct; identical to SEQ ID NO: 78

SEQ ID NO: 99 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 100 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 101 - protein, 116 aa, synthetic construct; identical to SEQ ID NO: 99

SEQ ID NO: 102 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 103 - protein, 116 aa, synthetic construct; identical to SEQ ID NO: 99

SEQ ID NO: 104 - protein, 111 aa, synthetic construct; identical to SEQ ID NO: 78

SEQ ID NO: 105 - protein, 116 aa, synthetic construct; identical to SEQ ID NO: 77

SEQ ID NO: 106 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 107 - protein, 116 aa, synthetic construct
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 108 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 109 - protein, 116 aa, synthetic construct; identical to SEQ ID NO: 77

SEQ ID NO: 110 - protein, 111 aa, synthetic construct
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 111 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 112 - protein, 111 aa, synthetic construct
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 113 - protein, 116 aa, synthetic construct
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 114 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 115 - protein, 116 aa, synthetic construct
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 116 - protein, 111 aa, synthetic construct
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 117 - protein, 116 aa, synthetic construct; identical to SEQ ID NO: 77

SEQ ID NO: 118 - protein, 111 aa, synthetic construct; identical to SEQ ID NO: 114

SEQ ID NO: 119 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 120 - protein, 111 aa, synthetic construct
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 121 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 122 - protein, 111 aa, synthetic construct
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 123 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 124 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 125 - protein, 116 aa, synthetic construct; identical to SEQ ID NO: 77

SEQ ID NO: 126 - protein, 111 aa, synthetic construct
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 127 - protein, 116 aa, synthetic construct; identical to SEQ ID NO: 77

SEQ ID NO: 128 - protein, 111 aa, synthetic construct
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 129 - protein, 116 aa, synthetic construct; identical to SEQ ID NO: 77

SEQ ID NO: 130 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 131 - protein, 116 aa, synthetic construct
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 132 - protein, 111 aa, synthetic construct
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 133 - protein, 116 aa, synthetic construct
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 134 - protein, 111 aa, synthetic construct; identical to SEQ ID NO: 114

SEQ ID NO: 135 - protein, 116 aa, synthetic construct; identical to SEQ ID NO: 77

SEQ ID NO: 136 - protein, 111 aa, synthetic construct; identical to SEQ ID NO: 124

SEQ ID NO: 137 - protein, 116 aa, synthetic construct; identical to SEQ ID NO: 107

SEQ ID NO: 138 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 139 - protein, 116 aa, synthetic construct
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 140 - protein, 111 aa, synthetic construct
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 141 - protein, 116 aa, synthetic construct; identical to SEQ ID NO: 107

SEQ ID NO: 142 - protein, 111 aa, synthetic construct; identical to SEQ ID NO: 114

SEQ ID NO: 143 - protein, 116 aa, synthetic construct; identical to SEQ ID NO: 113

SEQ ID NO: 144 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 145 - protein, 116 aa, synthetic construct; identical to SEQ ID NO: 77

SEQ ID NO: 146 - protein, 111 aa, synthetic construct; identical to SEQ ID NO: 138

SEQ ID NO: 147 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 148 - protein, 111 aa, synthetic construct
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 149 - protein, 116 aa, synthetic construct
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 150 - protein, 111 aa, synthetic construct
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 151 - protein, 116 aa, synthetic construct
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 152 - protein, 111 aa, synthetic construct
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 153 - protein, 116 aa, synthetic construct
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 154 - protein, 121 aa, synthetic construct
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 155 - protein, 127 aa, synthetic construct
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 156 - protein, 116 aa, synthetic construct
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 157 - protein, 116 aa, synthetic construct
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 158 - protein, 116 aa, synthetic construct
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 159 - protein, 116 aa, synthetic construct
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 160 - protein, 116 aa, synthetic construct
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 161 - protein, 116 aa, synthetic construct
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 162 - protein, 116 aa, synthetic construct
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 163 - protein, 116 aa, synthetic construct
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 164 - protein, 116 aa, synthetic construct
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 165 - protein, 116 aa, synthetic construct
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 166 - protein, 116 aa, synthetic construct
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 167 - protein, 116 aa, synthetic construct
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 168 - protein, 110 aa, synthetic construct
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 169 - protein, 110 aa, synthetic construct
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 170 - protein, 110 aa, synthetic construct
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 171 - protein, 110 aa, synthetic construct
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 172 - protein, 110 aa, synthetic construct
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 173 - protein, 110 aa, synthetic construct
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 174 - protein, 110 aa, synthetic construct
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 175 - protein, 110 aa, synthetic construct
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 176 - protein, 110 aa, synthetic construct
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 177 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 178 - protein, 110 aa, synthetic construct; identical to SEQ ID NO: 177

SEQ ID NO: 179 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 180 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 181 - protein, 110 aa, synthetic construct; identical to SEQ ID NO: 177

SEQ ID NO: 182 - protein, 111 aa, synthetic construct
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 183 - protein, 111 aa, synthetic construct
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 184 - protein, 111 aa, synthetic construct
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 185 - protein, 111 aa, synthetic construct
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 186 - protein, 111 aa, synthetic construct
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 187 - protein, 111 aa, synthetic construct
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 188 - protein, 117 aa, synthetic construct
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 189 - protein, 128 aa, synthetic construct
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 190 - protein, 122 aa, synthetic construct
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 191 - protein, 128 aa, synthetic construct
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 192 - protein, 117 aa, synthetic construct
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 193 - protein, 124 aa, synthetic construct
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 194 - protein, 117 aa, synthetic construct
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 195 - protein, 116 aa, synthetic construct
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 196 - protein, 117 aa, synthetic construct
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 197 - protein, 122 aa, synthetic construct
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 198 - protein, 107 aa, synthetic construct
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 199 - protein, 107 aa, synthetic construct
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 200 - protein, 107 aa, synthetic construct
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 201 - protein, 107 aa, synthetic construct
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 202 - protein, 107 aa, synthetic construct
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 203 - protein, 107 aa, synthetic construct
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 204 - protein, 107 aa, synthetic construct
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 205 - protein, 107 aa, synthetic construct
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 206 - protein, 107 aa, synthetic construct
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 207 - protein, 107 aa, synthetic construct
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK

SEQ ID NO: 208 - protein, 120 aa, synthetic construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVKVGYRGGMDVWGQGTTVTVSS

SEQ ID NO: 209 - protein, 120 aa, synthetic construct
EVQLLESGGGLVQPGGSLRLSCAASGFTLSSYAMHWVRQAPGKGLEYVSGISPSGSSTYYANSVKGRFTISRDNPKNTLFLQMSSLRAEDTAVYYCVRSKVRYRGGMDVWGQGTTVTVSS

SEQ ID NO: 210 - protein, 120 aa, synthetic construct
EVQLLESGGGLVQPGGSLRLSCAASGFTLSSYAMHWVRQAPGKGLEYVSGISPSGVSTYYANSVKGRFTISRDNPKNTLFLQMSSLRAEDTAVYYCVRVKVKYRGGMDVWGQGTTVTVSS

SEQ ID NO: 211 - protein, 120 aa, synthetic construct
EVQLLESGGGLVQPGGSLRLSCAASGFTLSSYAMHWVRQAPGKGLEYVSGISPTSGSTYYANSVKGRFTISRDNPKNTLFLQMSSLRAEDTAVYYCVRVKVRYRGGMDVWGQGTTVTVSS

SEQ ID NO: 212 - protein, 120 aa, synthetic construct
EVQLLESGGGLVQPGGSLRLSCAASGFTLSSYAMHWVRQAPGKGLEYVSGISPTGTSTYYANSVKGRFTISRDNPKNTLFLQMSSLRAEDTAVYYCVRVKGAYRGGMDVWGQGTTVTVSS

SEQ ID NO: 213 - protein, 120 aa, synthetic construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKVAYRGGMDVWGQGTTVTVSS

SEQ ID NO: 214 - protein, 120 aa, synthetic construct
EVQLLESGGGLVQPGGSLRLSCAASGFTLSSYAMHWVRQAPGKGLEYVSGISPSSTSTYYANSVKGRFTISRDNPKNTLFLQMSSLRAEDTAVYYCVRVKVLYRGGMDVWGQGTTVTVSS

SEQ ID NO: 215 - protein, 120 aa, synthetic construct
EVQLLESGGGLVQPGGSLRLSCAASGFTLSSYAMHWVRQAPGKGLEYVSGISPSSASTYYANSVKGRFTISRDNPKNTLFLQMSSLRAEDTAVYYCVRVKSKYRGGMDVWGQGTTVTVSS

SEQ ID NO: 216 - protein, 120 aa, synthetic construct
EVQLLESGGGLVQPGGSLRLSCAASGFTLSSYAMHWVRQAPGKGLEYVSGISGNSASTYYANSVKGRFTISRDNPKNTLFLQMSSLRAEDTAVYYCVRVKLKYRGGMDVWGQGTTVTVSS

SEQ ID NO: 217 - protein, 120 aa, synthetic construct
EVQLLESGGGLVQPGGSLRLSCAASGFTLSSYAMHWVRQAPGKGLEYVSGISHSGTSTYYANSVKGRFTISRDNPKNTLFLQMSSLRAEDTAVYYCVRVRVLYRGGMDVWGQGTTVTVSS

SEQ ID NO: 218 - protein, 120 aa, synthetic construct; identical to SEQ ID NO: 210

SEQ ID NO: 219 - protein, 120 aa, synthetic construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVFVRYRGGMDVWGQGTTVTVSS

SEQ ID NO: 220 - protein, 120 aa, synthetic construct
EVQLLESGGGLVQPGGSLRLSCAASGFTLSSYAMHWVRQAPGKGLEYVSGISPTSASTYYANSVKGRFTISRDNPKNTLFLQMSSLRAEDTAVYYCVRVKGRYRGGMDVWGQGTTVTVSS

SEQ ID NO: 221 - protein, 120 aa, synthetic construct
EVQLLESGGGLVQPGGSLRLSCAASGFTLSSYAMHWVRQAPGKGLEYVSGISPTGGSTYYANSVKGRFTISRDNPKNTLFLQMSSLRAEDTAVYYCVRVKGRYRGGMDVWGQGTTVTVSS

SEQ ID NO: 222 - protein, 120 aa, synthetic construct
EVQLLESGGGLVQPGGSLRLSCAASGFTLSSYAMHWVRQAPGKGLEYVSGISHSGNSTYYANSVKGRFTISRDNPKNTLFLQMSSLRAEDTAVYYCVRVKRRYRGGMDVWGQGTTVTVSS

SEQ ID NO: 223 - protein, 120 aa, synthetic construct
EVQLLESGGGLVQPGGSLRLSCAASGFTLSSYAMHWVRQAPGKGLEYVSGISPSSNSTYYANSVKGRFTISRDNPKNTLFLQMSSLRAEDTAVYYCVRVKVRYRGGMDVWGQGTTVTVSS

SEQ ID NO: 224 - protein, 120 aa, synthetic construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKPAYRGGMDVWGQGTTVTVSS

SEQ ID NO: 225 - protein, 120 aa, synthetic construct
EVQLLESGGGLVQPGGSLRLSCAASGFTLSSYAMHWVRQAPGKGLEYVSGISYSSASTYYANSVKGRFTISRDNPKNTLFLQMSSLRAEDTAVYYCVRVKVRYRGGMDVWGQGTTVTVSS

SEQ ID NO: 226 - protein, 108 aa, synthetic construct
ETTLTQSPDTLPLSPGDRASLSCRASQSVSSAYLAWYQQKPGQAPRLLIYGTSRRATGVPGRFSGSGSGTDFTLTISRLEPEDFAVYYCQLYGSSSVTFGQGTKLEIK

SEQ ID NO: 227 - protein, 108 aa, synthetic construct
EIVLTQSPGTLSLSPGERATLSCRASQGISSAYLAWYQQKPGQAPRLLIYGTSRRATGIPDRFSGSGSGTDFTLTISRLEPEDFAVYYCQLYGATSVTFGQGTKLEIK

SEQ ID NO: 228 - protein, 108 aa, synthetic construct; identical to SEQ ID NO: 227

SEQ ID NO: 229 - protein, 108 aa, synthetic construct
EIVLTQSPGTLSLSPGERATLSCRASQSVSSAYLAWYQQKPGQAPRLLIYGTSRRATGIPDRFSGSGSGTDFTLTISRLEPEDFAVYYCQLYGASSVTFGQGTKLEIK

SEQ ID NO: 230 - protein, 108 aa, synthetic construct; identical to SEQ ID NO: 229

SEQ ID NO: 231 - protein, 108 aa, synthetic construct; identical to SEQ ID NO: 227

SEQ ID NO: 232 - protein, 108 aa, synthetic construct; identical to SEQ ID NO: 229

SEQ ID NO: 233 - protein, 108 aa, synthetic construct
EIVLTQSPGTLSLSPGERATLSCRASQSISTAYLAWYQQKPGQAPRLLIYGTSRRATGIPDRFSGSGSGTDFTLTISRLEPEDFAVYYCQLYGASSVTFGQGTKLEIK

SEQ ID NO: 234 - protein, 108 aa, synthetic construct
EIVLTQSPGTLSLSPGERATLSCRASQDISSAYLAWYQQKPGQAPRLLIYGTSRRATGIPDRFSGSGSGTDFTLTISRLEPEDFAVYYCQLYGATSVTFGQGTKLEIK

SEQ ID NO: 235 - protein, 108 aa, synthetic construct
EIVLTQSPGTLSLSPGERATLSCRASQDVSSAYLAWYQQKPGQAPRLLIYGTSRRATGIPDRFSGSGSGTDFTLTISRLEPEDFAVYYCQLYGATSVTFGQGTKLEIK

SEQ ID NO: 236 - protein, 108 aa, synthetic construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGATSVTFGQGTKLEIK

SEQ ID NO: 237 - protein, 108 aa, synthetic construct
EIVLTQSPGTLSLSPGERATLSCRASQDASNAYLAWYQQKPGQAPRLLIYGTSRRATGIPDRFSGSGSGTDFTLTISRLEPEDFAVYYCQLYGSSSVTFGQGTKLEIK

SEQ ID NO: 238 - protein, 108 aa, synthetic construct
EIVLTQSPGTLSLSPGERATLSCRASQGVSSAYLAWYQQKPGQAPRLLIYGTSRRATGIPDRFSGSGSGTDFTLTISRLEPEDFAVYYCQLYGRSSVTFGQGTKLEIK

SEQ ID NO: 239 - protein, 108 aa, synthetic construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGTSSVTFGQGTKLEIK

SEQ ID NO: 240 - protein, 108 aa, synthetic construct
EIVLTQSPGTLSLSPGERATLSCRASQSVSTAYLAWYQQKPGQAPRLLIYGTSRRATGIPDRFSGSGSGTDFTLTISRLEPEDFAVYYCQLYGATSVTFGQGTKLEIK

SEQ ID NO: 241 - protein, 108 aa, synthetic construct; identical to SEQ ID NO: 234

SEQ ID NO: 242 - protein, 108 aa, synthetic construct
EIVLTQSPGTLSLSPGERATLSCRASQGVSTAYLAWYQQKPGQAPRLLIYGTSRRATGIPDRFSGSGSGTDFTLTISRLEPEDFAVYYCQLYGATSVTFGQGTKLEIK

SEQ ID NO: 243 - protein, 108 aa, synthetic construct
EIVLPQSPGTLSLSPGERATLSCRASQGVSSAYLAWYQQKPGQAPRLLIYGTSRRATGIPDRFSGSGSGTDFTLTISRLEPEDFAVYYCQLYGSTSVTFGQGTKLEIK

SEQ ID NO: 244 - protein, 118 aa, synthetic construct
EVQLVESGGGLVQPKGSLKLSCAASGFTFNTYGMHWVRQAPGKGLEWVAHIRSKSSNYATYYADSVKDRFTISRDDSQSMLYLQMNNLKTEDTAMYYCVRWFRAMDYWGQGTSVTVSS

SEQ ID NO: 245 - protein, 120 aa, synthetic construct
EVQLIESGGGLVQPKGSLKLSCAASGFTFNMYAMDWVRQAPGKGLEWVARIRSKGSNFETNYADSVKDRFTISRDDSQSMVYLQMINLKTEDTAMYYCVRHRGGAWFAYWGQGTLVSVSA

SEQ ID NO: 246 - protein, 121 aa, synthetic construct
QVQLVETGGGLVRPGNSLKLSCVTSGFTFSNYRMHWLRQPPGKRLEWIAVITVKSNNYGANYAESVKGRFAISRDDSKSSVYLEMNRLREEDTATYFCSRERAYGNPFDYWGQGTTLTVSS

SEQ ID NO: 247 - protein, 118 aa, synthetic construct
EVQLVESGGGLVQPKGSLKLSCAASGFTFNMYAMNWVRQAPGQGLEWVARIRSKSNNYATYYADSVKDRFIISRDDSESMVYLQMSNLRAADTAMYYCVRHLRAMDYWGQGTSVTVSS

SEQ ID NO: 248 - protein, 118 aa, synthetic construct
EVQLVESGGGLVQPKGSLKLSCAASGFSFNMYAMNWVRQAPGKGLEWVARIRTKSNHYSTYYADSVKDRFTISRDDSASMFYLQMNNLKTEDTAMYFCVRHLRAMDYWGQGTSVTVSS

SEQ ID NO: 249 - protein, 120 aa, synthetic construct
EVQLIESGGGLVQPKGSLKLSCAASGFTFNMYAMDWVRQAPGKGLEWVARIRSKGSNFETNYADSVKDRFTISRDDSQSMVYLQMNNLKTEDTAMYYCVRHRGGAWFAYWGQGTLVTVSA

SEQ ID NO: 250 - protein, 118 aa, synthetic construct
EVQLVESGGGLVRPKGSLKLSCAASGFSFNTYAMNWVRQAPGKGLEWIVWIRSKSHNYATYYADSVKDRFTISRDDSESMLYLQMNNLKTEDTAMYYCVRHLRAMDYWGQGTSVTVSS

SEQ ID NO: 251 - protein, 118 aa, synthetic construct
EVRLVESGGGLVQPKGSLKLSCEASGFSFNMYAMNWVRQAPGKGLEWITHIRSKSNNYATYYADSVKDRFIISRDDSESMVYLQMNNLKTEDTAMYYCVRLLRALDYWGQGTSVTVSS

SEQ ID NO: 252 - protein, 118 aa, synthetic construct
EVQLVESGGGLVQPKGSLKLSCAASGFTFNMYGMHWVRQAPGKGLEWVAHIRSKSSNYATYYADSVKDRLTISRDDSQSMLYLQMNNLKTEDTAMYYCVRWFRAMDYWGQGTSVTVSS

SEQ ID NO: 253 - protein, 118 aa, synthetic construct
EVQLVESGGGLVKPGGSLRLSCAASGFTFSMYGMHWVRQAPGKGLEWVGHIRSKSSNYATYYADSVKDRFTISRDDSKNTLYLQMNSLKTEDTAVYYCTTWFRAMDYWGQGTLVTVSS

SEQ ID NO: 254 - protein, 118 aa, synthetic construct
EVQLVESGGGLVQPGGSLRLSCAASGFTFSMYGMHWVRQAPGKGLEWVSHIRSKSSNYATYYADSVKDRFTISRDNAKNSLYLQMNSLRAEDTAVYYCARWFRAMDYWGQGTLVTVSS

SEQ ID NO: 255 - protein, 118 aa, synthetic construct
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTLVTVSS

SEQ ID NO: 256 - protein, 118 aa, synthetic construct
EVQLVESGGGLVQPGGSLRLSCAASGFTFSMYGMHWVRQAPGKGLEWVGHIRSKSSNYATYYADSVKDRFTISRDDSKNSLYLQMNSLKTEDTAVYYCARWFRAMDYWGQGTLVTVSS

SEQ ID NO: 257 - protein, 118 aa, synthetic construct; identical to SEQ ID NO: 254

SEQ ID NO: 258 - protein, 118 aa, synthetic construct
EVQLVESGGGLEQPGGSLRLSCAGSGFTFRMYGMHWVRQAPGKGLEWVSHIRSKSSNYATYYADSVKDRFTISRDNSKNTLYLQMNSLRAEDTAVYYCAKWFRAMDYWGQGTTVTVSS

SEQ ID NO: 259 - protein, 118 aa, synthetic construct
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTTVTVSS

SEQ ID NO: 260 - protein, 107 aa, synthetic construct
DIVMTQSHKFMSTSVGDRVSITCKASQDVSTAVAWYQEKPGQSPKLLIYWASTRHTGVPDRFTGSGSGTDYTLTISSVQAEDLALYYCQQHYSTPLTFGAGTKLELK

SEQ ID NO: 261 - protein, 111 aa, synthetic construct
DIVVTQSPASLAVSLGQRATISCRASKSVSTSGYSYMHWYQQKPGQPPKLLIYLASNLESGVPARFSGSGSGTDFTLNIHPVEEEDVAIYYCQHSRELPLTFGAGTKLELK

SEQ ID NO: 262 - protein, 107 aa, synthetic construct
DIQMTQSPSSLSASLGERVSLTCRASQEISGYLSWLQQKPDGTIKRLIYAASTLDSGVPKRFSGSRSGSDYSLTISSLESEDFADYYCLQYGSYPYTFGGGTKLEIK

SEQ ID NO: 263 - protein, 111 aa, synthetic construct
DIVLTQSPASLTVSLGQRATISCRASKSVSTSGYSYMHWYQQKPGQPPKLLIYLASNLESGVPARFSGSGSGTDFTLNIHPVEEEDAATYYCQHSRELPITFGSGTKLEIK

SEQ ID NO: 264 - protein, 111 aa, synthetic construct
DIVLTQSPASLVVSLGQRATISCRASQSVSTSGYSYMHWYQQKPGQPPKLLIYLASNVQSGVPARFSGSGSGTDFTLNIHPVEEEDVATYYCHHNRDLPFTFGSGTKLEIK

SEQ ID NO: 265 - protein, 111 aa, synthetic construct; identical to SEQ ID NO: 261

SEQ ID NO: 266 - protein, 111 aa, synthetic construct
DIVLTQSPASLAVSLGQRATISCRASKSVSASGYSYMHWYQQKPGQPPKLLIYLASNLQSGVPARFSGSGSGTDFTLNIHPVEEEDAATYYCQHSRELPPTFGGGTKLEIK

SEQ ID NO: 267 - protein, 111 aa, synthetic construct
DIVLTQSPASLAVFLGQRATISCRASKSVSTSGYSYMHWYQQKAGQPPKLLIYLASNLESGVPARFSGSGSGTDFTLNIHPVEEEDAATYYCHHSRELPITFGSGTKLEMK

SEQ ID NO: 268 - protein, 107 aa, synthetic construct; identical to SEQ ID NO: 260

SEQ ID NO: 269 - protein, 107 aa, synthetic construct
EIVLTQSPGTLSLSPGERATLSCKASQDVSTAVAWYQQKPGQAPRLLIYWASTRHTGIPDRFSGSGSGTDFTLTISRLEPEDFAVYYCQQHYSTPLTFGQGTKVEIK

SEQ ID NO: 270 - protein, 107 aa, synthetic construct
DIVMTQSPLSLPVTPGEPASISCKASQDVSTAVAWYLQKSGQSPQLLIYWASTRHTGVPDRFSGSGSGTDFTLKISRVEAEDVGFYYCQQHYSTPLTFGQGTKLEIK

SEQ ID NO: 271 - protein, 107 aa, synthetic construct
DIVMTQSPDSLAVSLGERATINCKASQDVSTAVAWYQQKPGQPPKLLIYWASTRHTGVPDRFSGSGSGTDFTLTISSLQAEDVAVYYCQQHYSTPLTFGGGTKVEIK

SEQ ID NO: 272 - protein, 107 aa, synthetic construct
DIQMTQSPSSLSASVGDRVTITCKASQDVSTAVAWYQQKPGKVPKLLIYWASTRHTGVPSRFSGSGSGTDFTLTISSLQPEDVATYYCQQHYSTPLTFGGGTKVEIK

SEQ ID NO: 273 - protein, 107 aa, synthetic construct
DIQLTQSPSFLSASVGDRVTITCKASQDVSTAVAWYQQKPGKAPKLLIYWASTRHTGVPSRFSGSGSGTEFTLTISSLQPEDFATYYCQQHYSTPLTFGGGTKVEIK

SEQ ID NO: 274 - protein, 107 aa, synthetic construct
DIQMTQSPSSLSASVGDRVTITCKASQDVSTAVAWYQQKPGKAPKLLLYWASTRHTGVPSRFSGSGSGTDYTLTISSLQPEDFATYYCQQHYSTPLTFGGGTKVEIK

SEQ ID NO: 275 - protein, 107 aa, synthetic construct
AIRMTQSPFSLSASVGDRVTITCKASQDVSTAVAWYQQKPAKAPKLFIYWASTRHTGVPSRFSGSGSGTDYTLTISSLQPEDFATYYCQQHYSTPLTFGGGTKVEIK

SEQ ID NO: 276 - protein, 117 aa, synthetic construct
EVQLQQSGPELVKPGASVRMSCKASGYTFTDYNIHWVKQSHGKSLEWIGYIYPNNGDNGYNQKFRGKATLTVDKSSSTAYMELRSLTSDDSAVYYCARGRLRYFDVWGTGTTVTVSS

SEQ ID NO: 277 - protein, 111 aa, synthetic construct
NIVLTQSPASLAVSLGQRATISCRASESVDNYGHSFMHWYQQKPGQPPKLLIYLASNLESGVPARFSGSGSRTDFTLTLDPVEADDAATYYCQQYNEDPPTFGSGTKLEIK

(19) United States
(12) Patent Application Publication
(10) Pub. No.: US 2024/0350626 A1
(43) Pub. Date: Oct. 24, 2024

(54) METHODS FOR TREATING EOSINOPHILIC GASTROENTERITIS BY ADMINISTERING AN IL-4R ANTAGONIST

(71) Applicant: REGENERON PHARMACEUTICALS, INC., Tarrytown, NY (US)

(72) Inventors: Seblewongel ASRAT, New York, NY (US); Siddhesh A. KAMAT, Jersey City, NJ (US); Xia LIU, Pleasantville, NY (US); Jennifer MALONEY, Hastings-on-Hudson, NY (US); Eilish C. MCCANN, Sloatsburg, NY (US); Jamie M. ORENGO, Cortlandt Manor, NY (US); George C. SCOTT, Glen Ridge, NJ (US); Arsalan Q. SHABBIR, Mount Kisco, NY (US)

(21) Appl. No.: 18/617,240

(22) Filed: Mar. 26, 2024

Related U.S. Application Data
(60) Provisional application No. 63/492,425, filed on Mar. 27, 2023.

(51) Int. Cl. A61K 39/395; A61K 31/569; A61K 31/573; A61K 31/58; A61P 1/00; A61P 37/06; C07K 16/28; A61K 39/00

(52) U.S. Cl. CPC A61K 39/3955; A61K 31/569; A61K 31/573; A61K 31/58; A61P 1/00; A61P 37/06; C07K 16/2866; A61K 2039/505

(57) ABSTRACT

Methods for treating eosinophilic gastritis or eosinophilic gastroenteritis are provided. In one aspect, the methods comprise administering to the subject one or more doses of an interleukin-4 receptor (IL-4R) antagonist, such as an anti-IL-4R antibody or antigen-binding fragment thereof.

### REFERENCE TO A SEQUENCE LISTING XML

This application contains a Sequence Listing which has been submitted electronically in XML format. The Sequence Listing XML is incorporated herein by reference. Said XML file, created on Mar. 14, 2024, is named 40848_0120USU1_SL.xml and is 269,767 bytes in size.

### CROSS-REFERENCE TO RELATED APPLICATIONS

This application claims priority to U.S. Provisional Patent Application No. 63/492,425, filed Mar. 27, 2023, the entire contents of which are incorporated by reference herein.

### FIELD OF THE INVENTION

The present disclosure relates to the use of interleukin-4 receptor (IL-4R) inhibitors to treat or prevent eosinophilic gastroenteritis, such as eosinophilic gastritis with or without eosinophilic duodenitis, in a subject in need thereof.

### BACKGROUND

Eosinophilic gastrointestinal disorders (EGIDs) are rare, chronic, allergic/immune mediated conditions that affect the gastrointestinal (GI) tract. EGIDs can occur in pediatric and adult patients and may either involve single regions of the gastrointestinal tract (i.e., esophagus, stomach, small bowel, and colon) or a combination of regions (Rossi, et al., Clin Trans/Allergy, 2022, 12:e12146).

Eosinophilic gastritis (EoG) is a rare disease of the stomach, distinct from eosinophilic esophagitis (EoE). EoG is characterized by patchy or diffuse infiltration of eosinophils in the stomach (Collins, et al., Front Med, 2017, 4:261). Patients with EoG may also have co-morbid eosinophilic duodenitis (EoD), which is characterized by patchy or diffuse infiltration of eosinophils in the small intestine. Mucosal biopsies from patients with EoG and with EoD show≥30 eosinophils (eos) per 5 high powered fields (hpfs) in the stomach and ≥30 eosinophils per 3 hpf in the small intestine, respectively; these are also used as diagnostic criteria for EoG and EoD, respectively (Dellon, et al., N Engl J Med, 2020, 383:1624-1634). Eosinophilic gastroenteritis (EGE) occurs within both the stomach and the small intestine and encompasses EoG and EoD.

Diagnosis of EoG with or without EoD is made based on clinical symptoms combined with eosinophilic infiltration in biopsy specimens from the stomach with or without eosinophilic inflammation in the duodenum and absence of other causes for the eosinophilia (e.g., malignancy, parasitic infections). The signs and symptoms of EoG with and without EoD are dependent on location, extent, and layer(s) of bowel involved with eosinophilic infiltration (Mendez Sànchez, et al., Dig Dis Sci, 2007, 52:2904-2911). The most frequent symptoms of eosinophilic infiltration of the mucosa are nausea, early satiety, vomiting, bloating, abdominal pain, abdominal cramping, loss of appetite, diarrhea, and weight loss. Patients with eosinophilic infiltration of the muscular layer may have symptoms of intestinal obstruction with nausea, vomiting, and abdominal distension. Patients with subserosal involvement may present with isolated ascites or ascites in combination with symptoms characteristic of mucosal and/or muscular disease. The majority of patients have a chronic course. The disease may involve deeper layers of the affected gastrointestinal tract, resulting in complications (e.g., intestinal obstruction) (Pineton de Chambrun, et al., Clin Gastroenterol Hepatol, 2011, 9:950-956).

Approximately 70% to 90% of patients with EoG (with or without EoD) have a history of an atopic/allergic disease which may include asthma, food allergies, atopic dermatitis, urticaria, allergic conjunctivitis, and/or allergic rhinitis/sinusitis (Chehade, et al., J Allergy Clin Immunol Pract, 2021, 9:2050-2059). Approximately 25% of patients have co-morbid EoE. Peripheral eosinophil counts are elevated in approximately 80% of patients, with peripheral eosinophil counts ranging from 5% to 35% with an average peripheral absolute eosinophil count of 1000 cells/μL (Chang, et al., Clin Gastroenterol Hepatol, 2010, 8:669-675).

Patients with EoG (with or without EoD) have a significantly impaired quality of life (QoL), and experience psychological, social, financial, and body image impacts. Patients were found to experience depression following development of their disease, emotional distress related to dietary restrictions, and financial impact due to their disease, either directly through cost of special foods, or indirectly through inability to work or go to school (Bedell, et al., Dig Sic Sci, 2018, 63:1148-1157).

There are no approved treatments for EoG (with or without EoD) in the US, Europe, or Japan. There are two main strategies for disease management of EoG (with or without EoD): diet modification and off-label pharmacologic interventions, predominantly systemic corticosteroids. Diet modification and/or pharmacological therapies may be effective for the treatment of some patients. However, maintenance of efficacy requires strict adherence to therapy and a restricted diet, and pharmacological therapies such as systemic corticosteroids are not a solution for long-term treatment due to their numerous systemic side effects. According, there remains an unmet need for safe and effective long-term therapies, which address the underlying inflammation of EoG (with or without EoD), prevent progression of disease, and improve clinical symptoms.

### SUMMARY

In one aspect, the present disclosure provides methods of treating, preventing, or ameliorating at least one symptom of eosinophilic gastroenteritis. In some embodiments, the method comprises administering to a subject having eosinophilic gastroenteritis one or more doses of an interleukin-4 receptor (IL-4R) antagonist.

In another aspect, the present disclosure provides methods of treating, preventing, or ameliorating at least one symptom of eosinophilic gastritis. In some embodiments, the method comprises administering to a subject having eosinophilic gastritis one or more doses of an IL-4R antagonist.

In another aspect, the present disclosure provides an interleukin-4 receptor (IL-4R) antagonist for use in treating, preventing, or ameliorating at least one symptom of eosinophilic gastroenteritis in a subject.

In another aspect, the present disclosure provides an IL-4R antagonist for use in treating, preventing, or ameliorating at least one symptom of eosinophilic gastritis in a subject.

In another aspect, the present disclosure provides an interleukin-4 receptor (IL-4R) antagonist for use in the preparation of a medicament for treating, preventing, or ameliorating at least one symptom of eosinophilic gastroenteritis in a subject.

In another aspect, the present disclosure provides an IL-4R antagonist for use in the preparation of a medicament for treating, preventing, or ameliorating at least one symptom of eosinophilic gastritis in a subject.

In another aspect, the present disclosure provides methods of reducing the use of a systemic corticosteroid or a swallowed topical corticosteroid in a subject having eosinophilic gastroenteritis by administering to the subject one or more doses of an interleukin-4 receptor (IL-4R) antagonist. In some embodiments, at the start of treatment with the IL-4R antagonist, the subject is on a stable dose of a maintenance systemic corticosteroid or swallowed topical corticosteroid. In some embodiments, treatment with the IL-4R antagonist reduces the subject's dependence on the systemic corticosteroid or swallowed topical corticosteroid. In some embodiments, treatment with the IL-4R antagonist eliminates the need for the systemic corticosteroid or swallowed topical corticosteroid.

In some embodiments, the subject has eosinophilic gastritis (EoG) with eosinophilic duodenitis (EoD). In some embodiments, the subject has EoG without EoD. In some embodiments, the subject has EoD without EoG. In some embodiments, the subject has eosinophilic gastroenteritis (e.g., EoG, EoG with EoD, or EoD) with esophageal involvement. In some embodiments, the subject has eosinophilic gastroenteritis (e.g., EoG, EoG with EoD, or EoD) and does not have eosinophilic esophagitis (EoE).

In some embodiments, the subject has been previously treated with a systemic corticosteroid or a swallowed topical corticosteroid. In some embodiments, the subject is unresponsive, inadequately responsive, or intolerant to treatment with a systemic corticosteroid or a swallowed topical corticosteroid, or wherein standard of care treatment is contraindicated.

In some embodiments, the subject is 12 years old. In some embodiments, the subject is an adult. In some embodiments, the subject is an adolescent.

In some embodiments, the subject has a concomitant atopic disease. In some embodiments, the concomitant atopic disease is a food allergy, atopic dermatitis, asthma, chronic rhinosinusitis, allergic rhinitis, or allergic conjunctivitis. In some embodiments, the subject has a concomitant atopic disease that is not eosinophilic esophagitis.

In some embodiments, prior to the onset of treatment with the IL-4R antagonist the subject has an eosinophil count 30 eos/hpf as measured by endoscopic biopsy in at least five distinct regions of the stomach, and/or an eosinophil count≥30 eos/hpf as measured by endoscopic biopsy in at least three distinct regions of the small intestine. In some embodiments, prior to the onset of treatment with the IL-4R antagonist the subject has a baseline total symptom score (TSS) of 20 as measured by the EoG/EoD Symptom Questionnaire. In some embodiments, prior to the onset of treatment with the IL-4R antagonist the subject has a baseline average severity score of 4 per week for at least two weeks for at least two components of the EoG/EoD-SQ, wherein the components are selected from the group consisting of stomach pain, stomach cramping, nausea, bloating, early satiety, and loss of appetite. In some embodiments, prior to the onset of treatment with the IL-4R antagonist the subject has a history of at least two episodes of EoG symptoms per week for at least 8 weeks, wherein the symptoms are selected from the group consisting of stomach pain, stomach cramping, nausea, bloating, early satiety, and loss of appetite.

In some embodiments, the IL-4R antagonist is an anti-IL-4R antibody or an antigen-binding fragment thereof. In some embodiments, the IL-4R antagonist is an anti-IL-4R antibody or an antigen-binding fragment thereof having one or more CDR, HCVR, and/or LCVR sequences listed in Table 1. In some embodiments, the IL-4R antagonist is an anti-IL-4R antibody, or an antigen-binding fragment thereof, that comprises three HCDRs (HCDR1, HCDR2 and HCDR3) and three LCDRs (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence of LGS, and the LCDR3 comprises the amino acid sequence of SEQ ID NO:8. In some embodiments, the anti-IL-4R antibody or antigen-binding fragment thereof comprises a heavy chain variable region (HCVR) comprising the amino acid sequence of SEQ ID NO:1 and comprises a light chain variable region (LCVR) comprising the amino acid sequence of SEQ ID NO:2. In some embodiments, wherein the anti-IL-4R antibody comprises a heavy chain comprising the amino acid sequence of SEQ ID NO:9 and a light chain comprising the amino acid sequence of SEQ ID NO:10. In some embodiments, the IL-4R antagonist is dupilumab.

In some embodiments, the IL-4R antagonist is administered at a dose of about 50 mg to about 600 mg. In some embodiments, the IL-4R antagonist is administered once a week, once every two weeks, once every three weeks, or once every four weeks. In some embodiments, the IL-4R antagonist is administered at a dose of about 300 mg QW. In some embodiments, the IL-4R antagonist is administered at a dose of about 300 mg Q2W. In some embodiments, the IL-4R antagonist is administered subcutaneously.

In some embodiments, the IL-4R antagonist is administered in combination with a second therapeutic agent or therapy.

In some embodiments, the IL-4R antagonist is contained in a container selected from the group consisting of a glass vial, a syringe, a pre-filled syringe, a pen delivery device, and an autoinjector. In some embodiments, the IL-4R antagonist is contained in a pre-filled syringe. In some embodiments, the pre-filled syringe is a single-dose pre-filled syringe. In some embodiments, the IL-4R antagonist is contained in an autoinjector. In some embodiments, the IL-4R antagonist is contained in a pen delivery device.

In another aspect, the present disclosure provides methods of treating a subject having eosinophilic gastroenteritis (e.g., EoG and/or EoD) by administering a combination therapy. In some embodiments, the combination therapy comprises (i) an interleukin-4 receptor (IL-4R) antagonist, and (ii) a systemic corticosteroid or a swallowed topical corticosteroid. In some embodiments, the combination therapy comprises an IL-4R antagonist as disclosed herein (e.g., an anti-IL-4R antibody or an antigen-binding fragment thereof, e.g., dupilumab) and a systemic corticosteroid (e.g., prednisone or prednisolone). In some embodiments, the combination therapy comprises an IL-4R antagonist as disclosed herein (e.g., an anti-IL-4R antibody or an antigen-binding fragment thereof, e.g., dupilumab) and a swallowed topical corticosteroid (e.g., budesonide or fluticasone).

In another aspect, the present disclosure provides a combination for treating a subject having eosinophilic gastroenteritis (e.g., EoG and/or EoD). In some embodiments, the combination comprises (i) an IL-4R antagonist, and (ii) a systemic corticosteroid or a swallowed topical corticosteroid. In some embodiments, the combination comprises an IL-4R antagonist as disclosed herein (e.g., an anti-IL-4R antibody or an antigen-binding fragment thereof, e.g., dupilumab) and a systemic corticosteroid (e.g., prednisone or prednisolone). In some embodiments, the combination comprises an IL-4R antagonist as disclosed herein (e.g., an anti-IL-4R antibody or an antigen-binding fragment thereof, e.g., dupilumab) and a swallowed topical corticosteroid (e.g., budesonide or fluticasone).

Other embodiments will be apparent from a review of the ensuing detailed description.

### BRIEF DESCRIPTION OF THE FIGURES

FIG. 1 shows that treatment with an IL-4R antagonist significantly reduced the frequency of eosinophils in stomach tissue of the mouse model of eosinophilic gastritis. Female C57BL/6J mice (n=5-7/group at study onset) were administered a single dose of 25 μg mIL-25 plasmid DNA or vector control plasmid DNA using HDD via IV injection on day 0. On days −4, 1, and 3, mice were administered 25 mg/kg REGN1103, isotype control antibody, or no Ab control via SC injection. Data are expressed as the group mean±SD, and symbols show data from individual animals. Normality was tested using Shapiro-Wilk test. Statistical significance was determined using Brown-Forsythe and Welch ANOVA tests and multiple comparisons done using Dunnett's T3 multiple comparison test. Across the study, 3 mice, no more than 2 per group, were excluded from analysis as they died prior to the conclusion of the experiment. *, p≤0.05; **, p≤0.01; ***, p≤0.001.

FIGS. 2A and 2B show that treatment with an IL-4R antagonist significantly reduced levels of Ccl11 and Ccl24 mRNA in the stomach tissue of a mouse model of eosinophilic gastritis. Female C57BL/6J mice (n=5-7/group at study onset) were administered a single dose of 25 μg mIL-25 plasmid DNA or vector control plasmid DNA using HDD via IV injection on day 0. On days −4, 1, and 3, mice were administered 25 mg/kg REGN1103, isotype control antibody, or no Ab control via SC injection. Samples were collected on day 8. Levels of Ccl11 (FIG. 2A) and Ccl24 (FIG. 2B) mRNA over Actb mRNA levels in stomach tissue were measured. Data are expressed as the group mean±SD, and symbols show data from individual animals. Normality was tested using Shapiro-Wilk test. P-values were determined using Kruskal-Wallis test and multiple comparisons done using Dunn's multiple comparisons test. Across the study, 3 mice, no more than 2 per group, were excluded from analysis as they died prior to the conclusion of the experiment. *, p≤0.05; **, p≤0.01.

FIGS. 3A and 3B show that treatment with an IL-4R antagonist significantly prevented pathology in a mouse model of eosinophilic gastritis. Female C57BL/6J mice (n=5-7/group at study onset) were administered a single dose of 25 μg mIL-25 plasmid DNA or vector control plasmid DNA using HDD via IV injection on day 0. On days −4, 1, and 3, mice were administered 25 mg/kg REGN1103, isotype control antibody, or no-antibody control via SC injection. Samples were collected on day 8, sectioned, and stained with hematoxylin and eosin. The group mean of the total pathology score from non-glandular and glandular stomach tissue (FIG. 3A) and representative images (FIG. 3B) for each group are shown. Data in panel A are expressed as the group mean of total pathology scores±SD, and symbols show data from individual animals. Statistical significance was determined using one-way ANOVA followed by Tukey's HSD test. The vertical horizontal line in images in panel B is a scale bar representing 2 mm in length. Across the study, 3 mice, no more than 2 per group, were excluded from analysis as they died prior to the conclusion of the experiment. **, p≤0.01; ***, p≤0.001; ****, p≤0.0001.

### DETAILED DESCRIPTION

### Definitions

Before the present invention is described, it is to be understood that the invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

“Eosinophilic gastritis,” or “EoG,” refers to an inflammatory disease characterized by abnormal eosinophilic inflammation in the stomach. The primary symptoms of EoG include, but are not limited to, nausea, early satiety, vomiting, bloating, abdominal pain, abdominal cramping, loss of appetite, diarrhea, and weight loss. EoG is typically diagnosed based on clinical symptoms combined with eosinophilic infiltration in biopsy specimens from the stomach. The current diagnostic criteria for EoG is 30 eosinophils (eos) per high powered fields (hpfs) in the stomach.

“Eosinophilic duodenitis,” or “EoD,” refers to an inflammatory disease characterized by patchy or diffuse infiltration of eosinophils in the small intestine. The primary symptoms of EoD include, but are not limited to, nausea, early satiety, vomiting, bloating, abdominal pain, abdominal cramping, loss of appetite, diarrhea, and weight loss. EoD is typically diagnosed based on clinical symptoms combined with eosinophilic infiltration in biopsy specimens from the small intestine. The current diagnostic criteria for EoD is 30 eosinophils (eos) per high powered fields (hpfs) in the small intestine.

As used herein, the term “subject in need thereof” refers to a human or a non-human animal that exhibits one or more symptoms or indications of eosinophilic gastroenteritis, and/or who has been diagnosed with eosinophilic gastroenteritis, e.g., EoG and/or EoD. In certain embodiments, the term includes subjects that show elevated levels of one or more EGE-associated biomarkers (described elsewhere herein). For example, in some embodiments a subject to be treated according to the methods of the disclosure is a subject with elevated levels of IgE, serum TARC, eotaxin-3, CCL11, or CCL24. As used herein, the terms “subject” and “patient” are used interchangeably.

The term “subject in need thereof” may also include, e.g., subjects who, prior to treatment, exhibit (or have exhibited) one or more indications of EGE such as eosinophilic infiltration of the gastrointestinal tract, nausea, early satiety, vomiting, bloating, abdominal pain, abdominal cramping, loss of appetite, diarrhea, weight loss, and/or an elevated level of a EGE-associated biomarker. The term also includes subjects with elevated peripheral eosinophil counts (e.g., ≥100, ≥150, ≥200, or ≥300 cells/μL) or elevated serum IgE (e.g., >150 kU/L).

The term “eosinophilic infiltration” refers to the presence of eosinophils in an organ or tissue including blood, esophagus, stomach, duodenum, jejunum, ileum, and colon of a subject. In the context of the present disclosure, the term “eosinophilic infiltration” refers to presence of eosinophils in the mucosal lining of a region of the gastrointestinal tract including, but not limited to, stomach and small intestine (e.g., in the mucosal lining of such region or regions).

Eosinophilic infiltration is analyzed, for example, using a tissue biopsy. According to some embodiments, “eosinophilic infiltration” refers to the presence of 30 eosinophils per high power field in the stomach, or in two, three, four, five or more distinct regions of the region (e.g., stomach or small intestine). The term “high power field” refers to a standard total magnification of 400× by a microscope used to view eosinophils in a tissue, e.g., from the stomach or small intestine of a subject. Thus, in some embodiments, a “subject in need thereof” refers to a subject who shows the presence of 30 eosinophils (“eos”) per high power field (“hpf”) in the gastrointestinal tract, e.g., in two, three, four, five or more of the proximal, mid, and distal regions of the stomach or small intestine. In certain embodiments, “eosinophilic infiltration” includes infiltration into a tissue by leukocytes, for example, lymphocytes, neutrophils and mast cells. The leukocyte infiltration into, e.g., gastrointestinal tissue can be detected by cell surface markers such macrophage-specific markers (e.g., CD11b⁺, F4/80⁺, CD14⁺, EMR1⁺, and CD68⁺), neutrophil-specific markers (e.g., CD11b⁺, Ly6G⁺, Ly6C⁺, CD11b⁺, and CD66b⁺), and T-cell-specific markers (e.g., CD3⁺, CD4⁺, and CD8⁺).

Although any methods and materials similar or equivalent to those described herein can be used in the practice of the disclosure, the typical methods and materials are now described. All publications mentioned herein are incorporated herein by reference in their entirety.

### Therapeutic Methods

In one aspect, methods for treating, preventing, or ameliorating one or more symptoms of eosinophilic gastroenteritis (EGE) in a subject are provided. In some embodiments, the subject to be treated has eosinophilic gastritis (EoG). In some embodiments, the subject to be treated has EoG with eosinophilic duodenitis (EoD). In some embodiments, the subject to be treated has EoG without EoD. In some embodiments, the subject to be treated has EoD without EoG. In some embodiments, the subject to be treated has EGE (e.g., EoG and/or EoD) with esophageal involvement. In some embodiments, the subject has concomitant eosinophilic esophagitis (EoE). In some embodiments, the subject does not have EoE.

In some embodiments, the subject is 12 years of age. In some embodiments, the subject is an adolescent. In some embodiments, the subject is an adult. In some embodiments, the subject is a pediatric subject<18 years of age, e.g., <12 years of age. In some embodiments, the subject has a body weight≥40 kg. In some embodiments, the subject is 12 years of age and has a body weight≥40 kg.

In some embodiments, a subject to be treated according to the methods disclosed herein has a history of documented diagnosis of EoG by endoscopic biopsy, as demonstrated by intraepithelial eosinophilic infiltration from at least 2, 3, 4, or 5 distinct regions of the stomach. In some embodiments, the subject has a baseline eosinophil count≥30 eos/hpf in at least 5 distinct regions of the stomach. “Eosinophil count,” as used herein, refers to the number of eosinophils contained within one high power field (hpf), e.g., at 400×. In some embodiments, the subject has a mean eosinophil count of ≥35 eos/hpf, ≥40 eos/hpf, ≥45 eos/hpf, ≥50 eos/hpf, ≥55 eos/hpf, ≥60 eos/hpf, ≥65 eos/hpf, ≥70 eos/hpf, ≥75 eos/hpf, ≥80 eos/hpf, ≥85 eos/hpf, or ≥90 eos/hpf.

In some embodiments, a subject to be treated according to the methods disclosed herein has a history of documented diagnosis of EoD by endoscopic biopsy, as demonstrated by intraepithelial eosinophilic infiltration from at least 2, 3, 4, or 5 distinct regions of the small intestine. In some embodiments, the subject has a baseline eosinophil count 30 eos/hpf in at least 3 distinct regions of the small intestine. In some embodiments, the subject has a mean eosinophil count of ≥35 eos/hpf, 40 eos/hpf, ≥45 eos/hpf, 50 eos/hpf, ≥55 eos/hpf, ≥60 eos/hpf, 65 eos/hpf, 70 eos/hpf, ≥75 eos/hpf, 80 eos/hpf, 85 eos/hpf, or ≥90 eos/hpf.

In some embodiments, a subject to be treated according to the methods disclosed herein has a history of one or more symptoms of EGE, EoG, or EoD, such as but not limited to, nausea, early satiety, vomiting, bloating, abdominal pain, abdominal cramping, loss of appetite, diarrhea, and weight loss. In some embodiments, the subject has a history of one or more symptoms of EGE for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 50 weeks, or longer. In some embodiments, the subject has a history of one or more symptoms of EGE, EoG, or EoD for at least 6 months, at least 1 year, at least 2 years, or longer.

In some embodiments, a subject to be treated according to the methods disclosed herein has a history of prior treatment with one or more standard-of-care therapies for EGE, such as but not limited to dietary modification (e.g., food-elimination diets), systemic corticosteroids, swallowed topical corticosteroids (e.g., oral beclomethasone, budesonide, or fluticasone), antihistamines, or immunomodulators. In some embodiments, a subject to be treated is a subject who is non-responsive, inadequately responsive, intolerant, or resistant to one or more of the current standard-of-care therapies for EGE, EoG, or EoD. In some embodiments, a subject to be treated has a contraindication for one or more standard-of-care therapies.

In some embodiments, a subject to be treated is on a stable dose of a maintenance systemic corticosteroid or swallowed topical corticosteroid. In some embodiments, a subject to be treated is on a stable dose of a PPI. In some embodiments, a subject to be treated is on a stable dose of a leukotriene inhibitor. In some embodiments, a subject to be treated is on a stable food elimination diet.

In some embodiments, a subject to be treated has, or has had, at least one comorbidity. In some embodiments, a subject to be treated has, or has had, a concomitant type 2 inflammatory condition. As used herein, a “type 2 inflammation condition” is a disease, disorder, or condition associated with a T helper 2 (T_{H}2)-mediated immune response (Gandhi, et al., Nat Rev Drug Discov., 2016, 15(1):35-50). Non-limiting examples of type 2 inflammatory conditions include asthma, chronic rhinosinusitis, allergic rhinitis, allergic fungal rhinosinusitis, chronic sinusitis, allergic bronchopulmonary aspergillosis (ABPA), unified airway disease, eosinophilic granulomatosis with polyangiitis (EGPA, formerly known as Churg-Strauss syndrome), atopic conjunctivitis, atopic dermatitis, vasculitis, cystic fibrosis (CF), chronic obstructive pulmonary disease (COPD), chronic rhinosinusitis with nasal polyps (CRSwNP), aspirin hypersensitivity, non-steroidal anti-inflammatory drug (NSAID) hypersensitivity (e.g., NSAIDs Exacerbated Respiratory Disease, or NSAID-ERD), perennial allergic rhinitis (PAR), chronic eosinophilic pneumonia (CEP) and exercise induced bronchospasm. In some embodiments, the subject has a concomitant atopic disease or condition selected from the group consisting of food allergy, atopic dermatitis, asthma, chronic rhinosinusitis, allergic rhinitis, or allergic conjunctivitis. In some embodiments, the subject has a concomitant atopic disease or condition that is not eosinophilic esophagitis.

In some embodiments, a subject to be treated is a subject who is susceptible to an allergen, e.g., a subject having a food allergy or oral allergy syndrome. For example, in some embodiments, the subject may exhibit one of the following characteristics: (a) is prone to allergic reactions or responses when exposed to one or more allergens; (b) has previously exhibited an allergic response or reaction to one or more allergens; (c) has a known history of allergies; and/or (d) exhibits a sign or symptom of an allergic response or anaphylaxis. As used herein, the phrases “allergic response,” “allergic reaction,” “allergic symptom,” and the like, include one or more signs or symptoms selected from the group consisting of urticaria (e.g., hives), angioedema, rhinitis, asthma, vomiting, sneezing, runny nose, sinus inflammation, watery eyes, wheezing, bronchospasm, reduced peak expiratory flow (PEF), gastrointestinal distress, flushing, swollen lips, swollen tongue, reduced blood pressure, anaphylaxis, and organ dysfunction/failure. An “allergic response,” “allergic reaction,” “allergic symptom,” etc., also includes immunological responses and reactions such as, e.g., increased IgE production, increased allergen-specific immunoglobulin production and/or eosinophilia. In some embodiments, the allergen is contained within or derived from a food item such as, e.g., dairy products (e.g., cow's milk), egg, wheat, soy, corn, rye, fish, shellfish, peanuts, tree nuts. In some embodiments, the allergen is contained within or derived from a non-food item such as, e.g., dust (e.g., containing dust mite), pollen, insect venom (e.g., venom of bees, wasps, mosquitoes, etc.), mold, animal dander, latex, medication, drugs, ragweed, grass, or birch.

In some embodiments, a subject to be treated is selected on the basis of exhibiting one or more inclusion criteria disclosed in Example 2. In some embodiments, a subject to be treated is further selected on the basis of not exhibiting one or more exclusion criteria disclosed in Example 2.

### Interleukin-4 Receptor Antagonists

In some embodiments, the methods of the present disclosure comprise administering to a subject in need thereof (e.g., a subject having EGE, EoG, or EoD) an interleukin-4 receptor (IL-4R) antagonist or a pharmaceutical composition comprising an IL-4R antagonist. As used herein, an “IL-4R antagonist” (also referred to herein as an “IL-4R inhibitor”, an “IL-4R blocker,” or an “IL-4Ra antagonist”) is any agent that binds to or interacts with IL-4Ra or an IL-4R ligand, and inhibits or attenuates the normal biological signaling function of a type 1 and/or a type 2 IL-4 receptor. Human IL-4Ra has the amino acid sequence of SEQ ID NO:11. A type 1 IL-4 receptor is a dimeric receptor comprising an IL-4Ra chain and a γc chain. A type 2 IL-4 receptor is a dimeric receptor comprising an IL-4Ra chain and an IL-13Rα1 chain. Type 1 IL-4 receptors interact with and are stimulated by IL-4, while type 2 IL-4 receptors interact with and are stimulated by both IL-4 and IL-13. Thus, the IL-4R antagonists that can be used in the methods of the present disclosure may function by blocking IL-4-mediated signaling, IL-13-mediated signaling, or both IL-4- and IL-13-mediated signaling. The IL-4R antagonists of the present disclosure may thus prevent the interaction of IL-4 and/or IL-13 with a type 1 or type 2 receptor.

Non-limiting examples of categories of IL-4R antagonists include small molecule IL-4R inhibitors, anti-IL-4R aptamers, peptide-based IL-4R inhibitors (e.g., “peptibody” molecules), “receptor-bodies” (e.g., engineered molecules comprising the ligand-binding domain of an IL-4R component), and antibodies or antigen-binding fragments of antibodies that specifically bind human IL-4Ra. As used herein, IL-4R antagonists also include antigen-binding proteins that specifically bind IL-4 and/or IL-13.

### Anti-IL-4Ra Antibodies and Antigen-Binding Fragments Thereof

In certain exemplary embodiments of the present disclosure, the IL-4R antagonist is an anti-IL-4Ra antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the anti-IL-4R antibody (or antigen-binding portion thereof) are identical to the human germline sequences. In some embodiments, one or more FRs of the anti-IL-4R antibody (or antigen-binding portion thereof) are naturally or artificially modified.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add, or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed by the term “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}—C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The constant region of an antibody is important in the ability of an antibody to fix complement and mediate cell-dependent cytotoxicity. Thus, in some embodiments the isotype of an antibody may be selected on the basis of whether it is desirable for the antibody to mediate cytotoxicity.

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, in some embodiments the methods of the present disclosure comprise the use of bispecific antibodies wherein one arm of an immunoglobulin is specific for IL-4Ra or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane, et al., J. Am. Chem. Soc., [Epub: Dec. 4, 2012]).

In some embodiments, the antibodies used in the methods of the present disclosure are human antibodies. The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present disclosure may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor, et al., (1992) Nucl. Acids Res., 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

According to certain embodiments, the antibodies used in the methods of the present disclosure specifically bind IL-4Ra. The term “specifically binds,” as used herein, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. In some embodiments, an antibody that “specifically binds” IL-4Rα binds to IL-4Rα or a portion thereof with an equilibrium dissociation constant (K_{D}) of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 0.5 nM, less than about 0.25 nM, less than about 0.1 nM or less than about 0.05 nM, as measured in a surface plasmon resonance assay (e.g., BIAcore™, Biacore Life Sciences division of GE Healthcare, Piscataway, NJ). In some embodiments, an antibody that specifically binds to a target antigen (e.g., IL-4Rα) can also specifically bind to another antigen, e.g., an ortholog of the target antigen. For example, in some embodiments, an isolated antibody that specifically binds human IL-4Rα exhibits cross-reactivity to other antigens, such as IL-4Rα molecules from other (non-human) species.

In some embodiments, the IL-4R antagonist is an anti-IL-4Rα antibody, or antigen-binding fragment thereof, comprising a heavy chain variable region (HCVR), light chain variable region (LCVR), and/or complementarity determining regions (CDRs) comprising any of the amino acid sequences of the anti-IL-4R antibodies as set forth in U.S. Pat. No. 7,608,693, incorporated by reference herein. In some embodiments, the IL-4R antagonist is an anti-IL-4Rα antibody or antigen-binding fragment thereof that comprises the heavy chain complementarity determining regions (HCDRs) of a heavy chain variable region (HCVR) comprising the amino acid sequence of SEQ ID NO:1 and the light chain complementarity determining regions (LCDRs) of a light chain variable region (LCVR) comprising the amino acid sequence of SEQ ID NO:2. In some embodiments, the IL-4R antagonist is an anti-IL-4Rα antibody or antigen-binding fragment thereof that comprises three HCDRs (HCDR1, HCDR2, and HCDR3) and three LCDRs (LCDR1, LCDR2, and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence of LGS, and the LCDR3 comprises the amino acid sequence of SEQ ID NO:8.

In some embodiments, the anti-IL-4R antibody or antigen-binding fragment thereof comprises the HCDR1, HCDR2, HCDR3, LCDR1, LCDR2, and LCDR3 of SEQ ID NO:3, SEQ ID NO:4, SEQ ID NO:5, SEQ ID NO:6, LGS, and SEQ ID NO:8, respectively, and further comprises an HCVR having at least 85% sequence identity (e.g., at least 90%, 91%, 92%, 93%, 94%, 95%, 96%, 97%, 98%, or 99% sequence identity) to the amino acid sequence of SEQ ID NO:1 and an LCVR having at least 85% sequence identity (e.g., at least 90%, 91%, 92%, 93%, 94%, 95%, 96%, 97%, 98%, or 99% sequence identity) to the amino acid sequence of SEQ ID NO:2. In some embodiments, the anti-IL-4R antibody or antigen-binding fragment thereof comprises an HCVR comprising SEQ ID NO:1 and an LCVR comprising SEQ ID NO:2.

In some embodiments, the anti-IL-4R antibody comprises a heavy chain comprising the amino acid sequence of SEQ ID NO:9. In some embodiments, the anti-IL-4R antibody comprises a light chain comprising the amino acid sequence of SEQ ID NO:10.

An exemplary antibody comprising a heavy chain comprising the amino acid sequence of SEQ ID NO:9 and a light chain comprising the amino acid sequence of SEQ ID NO:10 is the fully human anti-IL-4R antibody known as dupilumab. According to certain exemplary embodiments, the methods of the present disclosure comprise the use of dupilumab. As used herein, “dupilumab” also includes bioequivalents of dupilumab. The term “bioequivalent,” as used herein with reference to dupilumab, refers to anti-IL-4R antibodies or IL-4R-binding proteins or fragments thereof that are pharmaceutical equivalents or pharmaceutical alternatives whose rate and/or extent of absorption do not show a significant difference with that of dupilumab when administered at the same molar dose under similar experimental conditions, either single dose or multiple doses. In some embodiments, the term refers to antigen-binding proteins that bind to IL-4R which do not have clinically meaningful differences with dupilumab in their safety, purity and/or potency.

Other anti-IL-4Rα antibodies that can be used in the context of the methods of the present disclosure include, e.g., the antibody referred to and known in the art as AMG317 (Corren, et al., 2010, Am J Respir Crit Care Med., 181(8):788-796), or MEDI 9314, or any of the anti-IL-4Rα antibodies as set forth in U.S. Pat. Nos. 7,186,809, 7,605,237, 7,638,606, 8,092,804, 8,679,487, 8,877,189, 10,774,141, or International Patent Publication Nos. WO2020/096381, WO 2020/182197, WO2020/239134, WO 2021/213329, WO2022/052974, WO2022/136669, or WO2022/136675, the contents of each of which are incorporated by reference herein.

In some embodiments, an anti-IL-4Rα antibody or antigen-binding fragment thereof for use in the methods of the present disclosure comprises one or more CDR, HCVR, and/or LCVR sequences set forth in Table 1 below.

In some embodiments, an anti-IL-4Rα antibody comprises (i) an HCVR comprising the amino acid sequence of SEQ ID NO:32 (SCB-VH-59), SEQ ID NO:33 (SCB-VH-60), SEQ ID NO:34 (SCB-VH-61), SEQ ID NO:35 (SCB-VH-62), SEQ ID NO:36 (SCB-VH-63), SEQ ID NO:37 (SCB-VH-64), SEQ ID NO:38 (SCB-VH-65), SEQ ID NO:39 (SCB-VH-66), SEQ ID NO:40 (SCB-VH-67), SEQ ID NO:41 (SCB-VH-68), SEQ ID NO:42 (SCB-VH-69), SEQ ID NO:43 (SCB-VH-70), SEQ ID NO:44 (SCB-VH-71), SEQ ID NO:45 (SCB-VH-72), SEQ ID NO:46 (SCB-VH-73), SEQ ID NO:47 (SCB-VH-74), SEQ ID NO:48 (SCB-VH-75), SEQ ID NO:49 (SCB-VH-76), SEQ ID NO:50 (SCB-VH-77), SEQ ID NO:51 (SCB-VH-78), SEQ ID NO:52 (SCB-VH-79), SEQ ID NO:53 (SCB-VH-80), SEQ ID NO:54 (SCB-VH-81), SEQ ID NO:55 (SCB-VH-82), SEQ ID NO:56 (SCB-VH-83), SEQ ID NO:57 (SCB-VH-84), SEQ ID NO:58 (SCB-VH-85), SEQ ID NO:59 (SCB-VH-86), SEQ ID NO:60 (SCB-VH-87), SEQ ID NO:61 (SCB-VH-88), SEQ ID NO:62 (SCB-VH-89), SEQ ID NO:63 (SCB-VH-90), SEQ ID NO:64 (SCB-VH-91), SEQ ID NO:65 (SCB-VH-92), or SEQ ID NO:66 (SCB-VH-93); and (ii) an LCVR comprising the amino acid sequence of SEQ ID NO:12 (SCB-VL-39), SEQ ID NO:13 (SCB-VL-40), SEQ ID NO:14 (SCB-VL-41), SEQ ID NO:15 (SCB-VL-42), SEQ ID NO:16 (SCB-V_{L}-43), SEQ ID NO:17 (SCB-VL-44), SEQ ID NO:18 (SCB-VL-45), SEQ ID NO:19 (SCB-VL-46), SEQ ID NO:20 (SCB-VL-47), SEQ ID NO:21 (SCB-VL-48), SEQ ID NO:22 (SCB-VL-49), SEQ ID NO:23 (SCB-VL-50), SEQ ID NO:24 (SCB-VL-51), SEQ ID NO:25 (SCB-VL-52), SEQ ID NO:26 (SCB-VL-53), SEQ ID NO:27 (SCB-VL-54), SEQ ID NO:28 (SCB-VL-55), SEQ ID NO:29 (SCB-VL-56), SEQ ID NO:30 (SCB-VL-57), or SEQ ID NO:31 (SCB-VL-58). In some embodiments, the anti-IL-4Rα antibody comprises an HCVR comprising the amino acid sequence of SEQ ID NO:64 (SCB-VH-91) and an LCVR comprising the amino acid sequence of SEQ ID NO:17 (SCB-VL-44), SEQ ID NO:27 (SCB-VL-54), or SEQ ID NO:28 (SCB-VL-55).

In some embodiments, an anti-IL-4Rα antibody comprises an amino acid sequence pair selected from the group consisting of: SEQ ID NOs:67/68 (MEDI-1-VH/MEDI-1-VL); SEQ ID NOs:69/70 (MEDI-2-VH/MEDI-2-VL); SEQ ID NOs:71/72 (MEDI-3-VH/MEDI-3-VL); SEQ ID NOs:73/74 (MEDI-4-VH/MEDI-4-VL); SEQ ID NOs:75/76 (MEDI-5-VH/MEDI-5-VL); SEQ ID NOs:77/78 (MEDI-6-VH/MEDI-6/VL); SEQ ID NOs:79/80 (MEDI-7-VH/MEDI-7-VL); SEQ ID NOs:81/82 (MEDI-8-VH/MEDI-8-VL); SEQ ID NOs:83/84 (MEDI-9-VH/MEDI-9-VL); SEQ ID NOs:85/86 (MEDI-10-VH/MEDI-10-VL); SEQ ID NOs:87/88 (MEDI-11-VH/MEDI-11/VL); SEQ ID NOs:89/90 (MEDI-12-VH/MEDI-12-VL); SEQ ID NOs:91/92 (MEDI-13-VH/MEDI-13-VL); SEQ ID NOs:93/94 (MEDI-14-VH/MEDI-14-VL); SEQ ID NOs:95/96 (MEDI-15-VH/MEDI-15-VL); SEQ ID NOs:97/98 (MEDI-16-VH/MEDI-16/VL); SEQ ID NOs:99/100 (MEDI-17-VH/MEDI-17-VL); SEQ ID NOs:101/102 (MEDI-18-VH/MEDI-18-VL); SEQ ID NOs:103/104 (MEDI-19-VH/MEDI-19-VL); SEQ ID NOs:105/106 (MEDI-20-VH/MEDI-20-VL); SEQ ID NOs:107/108 (MEDI-21-VH/MEDI-21-VL); SEQ ID NOs:109/110 (MEDI-22-VH/MEDI-22-VL); SEQ ID NOs:111/112 (MEDI-23-VH/MEDI-23-VL); SEQ ID NOs:113/114 (MEDI-24-VH/MEDI-24-VL); SEQ ID NOs:115/116 (MEDI-25-VH/MEDI-25-VL); SEQ ID NOs:117/118 (MEDI-26-VH/MEDI-26-VL); SEQ ID NOs:119/120 (MEDI-27-VH/MEDI-27-VL); SEQ ID NOs:121/122 (MEDI-28-VH/MEDI-28-VL); SEQ ID NOs:123/124 (MEDI-29-VH/MEDI-29-VL); SEQ ID NOs:125/126 (MEDI-30-VH/MEDI-30-VL); SEQ ID NOs:127/128 (MEDI-31-VH/MEDI-31-VL); SEQ ID NOs:129/130 (MEDI-32-VH/MEDI-32-VL); SEQ ID NOs:131/132 (MEDI-33-VH/MEDI-33-VL); SEQ ID NOs:133/134 (MEDI-34-VH/MEDI-34-VL); SEQ ID NOs:135/136 (MEDI-35-VH/MEDI-35-VL); SEQ ID NOs:137/138 (MEDI-36-VH/MEDI-36-VL); SEQ ID NOs:139/140 (MEDI-37-VH/MEDI-37-VL); SEQ ID NOs:141/142 (MEDI-38-VH/MEDI-38-VL); SEQ ID NOs:143/144 (MEDI-39-VH/MEDI-39-VL); SEQ ID NOs:145/146 (MEDI-40-VH/MEDI-40-VL); SEQ ID NOs:147/148 (MEDI-41-VH/MEDI-41-VL); SEQ ID NOs:149/150 (MEDI-42-VH/MEDI-42-VL); and SEQ ID NOs:151/152 (MEDI-37GL-VH/MEDI-37GL-VL).

In some embodiments, an anti-IL-4Rα antibody comprises (i) an HCVR comprising the amino acid sequence of SEQ ID NO:153 (AJOU-1-VH), SEQ ID NO:154 (AJOU-2-VH), SEQ ID NO:155 (AJOU-3-VH), SEQ ID NO:156 (AJOU-4-VH), SEQ ID NO:157 (AJOU-5-VH), SEQ ID NO:158 (AJOU-6-VH), SEQ ID NO:159 (AJOU-7-VH), SEQ ID NO:160 (AJOU-8-VH), SEQ ID NO:161 (AJOU-9-VH), SEQ ID NO:162 (AJOU-10-VH), SEQ ID NO:163 (AJOU-69-VH), SEQ ID NO:164 (AJOU-70-VH), SEQ ID NO:165 (AJOU-71-VH), SEQ ID NO:166 (AJOU-72-VH), or SEQ ID NO:167 (AJOU-83-VH); and (ii) an LCVR comprising the amino acid sequence of SEQ ID NO:168 (AJOU-33-VL), SEQ ID NO:169 (AJOU-34-VL), SEQ ID NO:170 (AJOU-35-VL), SEQ ID NO:171 (AJOU-36-VL), SEQ ID NO:172 (AJOU-37-VL), SEQ ID NO:173 (AJOU-38-VL), SEQ ID NO:174 (AJOU-39-VL), SEQ ID NO:175 (AJOU-40-VL), SEQ ID NO:176 (AJOU-41-VL), SEQ ID NO:177 (AJOU-42-VL), SEQ ID NO:178 (AJOU-77-VL), SEQ ID NO:179 (AJOU-78-VL), SEQ ID NO:180 (AJOU-79-VL), SEQ ID NO:181 (AJOU-80-VL), SEQ ID NO:182 (AJOU-86-VL), SEQ ID NO:183 (AJOU-87-VL), SEQ ID NO:184 (AJOU-88-VL), SEQ ID NO:185 (AJOU-89-VL), SEQ ID NO:186 (AJOU-90-VL), or SEQ ID NO:187 (AJOU-91-VL).

In some embodiments, an anti-IL-4Rα antibody comprises (i) an HCVR comprising the amino acid sequence of SEQ ID NO:188 (REGN-VH-3), SEQ ID NO:189 (REGN-VH-19), SEQ ID NO:190 (REGN-VH-35), SEQ ID NO:191 (REGN-VH-51), SEQ ID NO:192 (REGN-VH-67), SEQ ID NO:193 (REGN-VH-83), SEQ ID NO:194 (REGN-VH-99), SEQ ID NO:195 (REGN-VH-115), SEQ ID NO:196 (REGN-VH-147), or SEQ ID NO:197 (REGN-VH-163); and (ii) an LCVR comprising the amino acid sequence of SEQ ID NO:198 (REGN-VL-11), SEQ ID NO:199 (REGN-VL-27), SEQ ID NO:200 (REGN-VL-43), SEQ ID NO:201 (REGN-VL-59), SEQ ID NO:202 (REGN-VL-75), SEQ ID NO:203 (REGN-VL-91), SEQ ID NO:204 (REGN-VL-107), SEQ ID NO:205 (REGN-VL-123), SEQ ID NO:206 (REGN-VL-155), or SEQ ID NO:207 (REGN-VL-171).

In some embodiments, an anti-IL-4Rα antibody comprises (i) an HCVR comprising the amino acid sequence of SEQ ID NO:208 (STSA-C27-VH), SEQ ID NO:209 (STSA-C27-6-33-VH), SEQ ID NO:210 (STSA-C27-7-33-VH), SEQ ID NO:211 (STSA-C27-24-56-VH), SEQ ID NO:212 (STSA-C27-47-56-VH), SEQ ID NO:213 (STSA-C27-33-33-VH), SEQ ID NO:214 (STSA-C27-56-56-VH), SEQ ID NO:215 (STSA-C27-78-78-VH), SEQ ID NO:216 (STSA-C27-82-58-VH), SEQ ID NO:217 (STSA-C27-54-54-VH), SEQ ID NO:218 (STSA-C27-36-36-VH), SEQ ID NO:219 (STSA-C27-53-53-VH), SEQ ID NO:220 (STSA-C27-67-67-VH), SEQ ID NO:221 (STSA-C27-55-55-VH), SEQ ID NO:222 (STSA-C27-59-59-VH), SEQ ID NO:223 (STSA-C27-58-58-VH), SEQ ID NO:224 (STSA-C27-52-52-VH), or SEQ ID NO:225 (STSA-C27-Y2-Y2-VH); and (ii) an LCVR comprising the amino acid sequence of SEQ ID NO:226 (STSA-C27-VL), SEQ ID NO:227 (STSA-C27-6-33-VL), SEQ ID NO:228 (STSA-C27-7-33-VL), SEQ ID NO:229 (STSA-C27-24-56-VL), SEQ ID NO:230 (STSA-C27-47-56-VL), SEQ ID NO:231 (STSA-C27-33-33-VL), SEQ ID NO:232 (STSA-C27-56-56-VL), SEQ ID NO:233 (STSA-C27-78-78-VL), SEQ ID NO:234 (STSA-C27-82-58-VL), SEQ ID NO:235 (STSA-C27-54-54-VL), SEQ ID NO:236 (STSA-C27-36-36-VL), SEQ ID NO:237 (STSA-C27-53-53-VL), SEQ ID NO:238 (STSA-C27-67-67-VL), SEQ ID NO:239 (STSA-C27-55-55-VL), SEQ ID NO:240 (STSA-C27-59-59-VL), SEQ ID NO:241 (STSA-C27-58-58-VL), SEQ ID NO:242 (STSA-C27-52-52-VL), or SEQ ID NO:243 (STSA-C27-Y2-Y2-VL).

In some embodiments, an anti-IL-4Rα antibody comprises (i) an HCVR comprising the amino acid sequence of SEQ ID NO:244 (Y0188-1 VH), SEQ ID NO:245 (Y0188-2 VH), SEQ ID NO:246 (Y0188-3 VH), SEQ ID NO:247 (Y0188-4 VH), SEQ ID NO:248 (Y0188-6 VH), SEQ ID NO:249 (Y0188-8 VH), SEQ ID NO:250 (Y0188-9 VH), SEQ ID NO:251 (Y0188-10 VH), SEQ ID NO:252 (Y0188-14 VH), SEQ ID NO:253 (HV3-15-14 VH), SEQ ID NO:254 (HV3-48-14 VH), SEQ ID NO:255 (HV3-73*2-14 VH), SEQ ID NO:256 (HV3-72-14 VH), SEQ ID NO:257 (Y01-14 VH), SEQ ID NO:258 (162-14 VH), or SEQ ID NO:259 (VH73-14 VH); and (ii) an LCVR comprising the amino acid sequence of SEQ ID NO:260 (Y0188-1 VL), SEQ ID NO:261 (Y0188-2 VL), SEQ ID NO:262 (Y0188-3 VL), SEQ ID NO:263 (Y0188-4 VL), SEQ ID NO:264 (Y0188-6 VL), SEQ ID NO:265 (Y0188-8 VL), SEQ ID NO:266 (Y0188-9 VL), SEQ ID NO:267 (Y0188-10 VL), SEQ ID NO:268 (Y0188-14 VL), SEQ ID NO:269 (Y01-14 VL), SEQ ID NO:270 (164-14 VL), SEQ ID NO:271 (KV4-14 VL), SEQ ID NO:272 (KV1-27-14 VL), SEQ ID NO:273 (KV1-9-14 VL), SEQ ID NO:274 (KV1-NL1-14 VL), or SEQ ID NO:275 (KV1 D-43-14 VL).

In some embodiments, an anti-IL-4Rα antibody used in the methods of the present disclosure can have pH-dependent binding characteristics. For example, an anti-IL-4Rα antibody for use as disclosed herein may exhibit reduced binding to IL-4Rα at acidic pH as compared to neutral pH. Alternatively, an anti-IL-4Rα antibody for use as disclosed herein may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to IL-4Rα at acidic pH as compared to neutral pH” is expressed in terms of a ratio of the K_{D} value of the antibody binding to IL-4Rα at acidic pH to the K_{D} value of the antibody binding to IL-4Rα at neutral pH (or vice versa). For example, an antibody or antigen-binding fragment thereof may be regarded as exhibiting “reduced binding to IL-4Rα at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

### Preparation of Human Antibodies

Methods for generating human antibodies in transgenic mice are known in the art. Any such known methods can be used in the context of the present disclosure to make human antibodies that specifically bind to human IL-4R.

Using VELOCIMMUNE™ technology (see, for example, U.S. Pat. No. 6,596,541, Regeneron Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate the fully human antibody of the disclosure, for example wild-type or modified IgG1 or IgG4. While the constant region selected may vary according to specific use, high affinity antigen-binding and target specificity characteristics reside in the variable region.

In general, the antibodies that can be used in the methods of the present disclosure possess high affinities, as described above, when measured by binding to antigen either immobilized on solid phase or in solution phase. The mouse constant regions are replaced with desired human constant regions to generate the fully human antibodies of the disclosure. While the constant region selected may vary according to specific use, high affinity antigen-binding and target specificity characteristics reside in the variable region.

In one embodiment, a human antibody or antigen-binding fragment thereof that specifically binds IL-4R and that can be used in the methods disclosed herein comprises the three heavy chain CDRs (HCDR1, HCDR2, and HCDR3) contained within a heavy chain variable region (HCVR) having an amino acid sequence of SEQ ID NO:1, and the three light chain CDRs (LCVR1, LCVR2, and LCVR3) contained within a light chain variable region (LCVR) having an amino acid sequence of SEQ ID NO:2. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani, et al., J. Mol. Biol., 273:927-948 (1997); and Martin, et al., Proc. Natl. Acad. Sci., USA 86:9268-9272 (1989). Public databases are also available for identifying CDR sequences within an antibody.

### Pharmaceutical Compositions and Kits

In one aspect, the present disclosure provides methods that comprise administering an IL-4R antagonist to a subject, wherein the IL-4R antagonist (e.g., an anti-IL-4R antibody) is contained within a pharmaceutical composition that comprises one or more pharmaceutically acceptable vehicle, carriers, and/or excipients. Various pharmaceutically acceptable carriers and excipients are well-known in the art. See, e.g., Remington's Pharmaceutical Sciences, Mack Publishing Company, Easton, PA. In some embodiments, the carrier is suitable for intravenous, intramuscular, oral, intraperitoneal, intrathecal, transdermal, topical, or subcutaneous administration.

Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously.

In some embodiments, the pharmaceutical composition comprises an injectable preparation, such as a dosage form for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The dose of antibody administered to a subject according to the methods of the present disclosure may vary depending upon the age and the size of the subject, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-4R antibodies may be determined empirically; for example, subject progress can be monitored by periodic assessment, and the dose adjusted accordingly. Moreover, interspecies scaling of dosages can be performed using well-known methods in the art (e.g., Mordenti, et al., 1991, Pharmaceut. Res., 8:1351). Specific exemplary doses of anti-IL-4R antibodies, and administration regimens involving the same, that can be used in the context of the present disclosure are disclosed elsewhere herein.

In some embodiments, an IL-4R antagonist or a pharmaceutical composition of the present disclosure is contained within a container. Thus, in another aspect, containers comprising an IL-4R antagonist or a pharmaceutical composition as disclosed herein are provided. For example, in some embodiments, a pharmaceutical composition is contained within a container selected from the group consisting of a glass vial, a syringe, a pen delivery device, and an autoinjector.

In some embodiments, a pharmaceutical composition of the present disclosure is delivered, e.g., subcutaneously or intravenously, with a standard needle and syringe. In some embodiments, the syringe is a pre-filled syringe. In some embodiments, a pen delivery device or autoinjector is used to deliver a pharmaceutical composition of the present disclosure (e.g., for subcutaneous delivery). A pen delivery device can be reusable or disposable. Typically, a reusable pen delivery device utilizes a replaceable cartridge that contains a pharmaceutical composition. Once the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™ OPTIPEN STARLET™, and OPTICLIK™ (sanofi-aventis, Frankfurt, Germany). Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL).

In some embodiments, the pharmaceutical composition is delivered using a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science, 249:1527-1533. Other delivery systems are known and can be used to administer the pharmaceutical composition, e.g., encapsulation in liposomes, microparticles, microcapsules, recombinant cells capable of expressing the mutant viruses, receptor mediated endocytosis (see, e.g., Wu, et al., 1987, J. Biol. Chem. 262:4429-4432).

In some embodiments, a pharmaceutical composition comprising an anti-IL-4R antibody is administered using a drug delivery device that is a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user).

Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

An exemplary sleeve-triggered auto-injector with manual needle insertion is described in International Publication WO2015/004052. Exemplary audible end-of-dose feedback mechanisms are described in International Publications WO2016/193346 and WO2016/193348. An exemplary needle-safety mechanism after using an auto-injector is described in International Publication WO2016/193352. An exemplary needle sheath remover mechanism for a syringe auto-injector is described in International Publication WO2016/193353. An exemplary support mechanism for supporting an axial position of a syringe is described in International Publication WO2016/193355.

In some embodiments, pharmaceutical compositions for use as described herein are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used in the context of the present disclosure are disclosed, e.g., in U.S. Pat. No. 8,945,559.

In another aspect, kits comprising an IL-4R antagonist or pharmaceutical composition as disclosed herein are provided. In some embodiments, the kit comprises an anti-IL-4R antibody, or a pharmaceutical composition comprising an anti-IL-4R antibody, and instructions for the use thereof in treating eosinophilic gastroenteritis in a subject. In some embodiments, the instructions for use comprise administering the anti-IL-4R antibody or pharmaceutical composition in an amount, dosing frequency, and/or for a length of time as disclosed elsewhere herein.

### Dosage and Administration

In some embodiments, an IL-4R antagonist (e.g., anti-IL-4R antibody) is administered to a subject (e.g., a subject having EGE, EoG, or EoD) according to the methods of the present disclosure in a therapeutically effective amount. As used herein with reference to an IL-4R antagonist, the phrase “therapeutically effective amount” means an amount of IL-4R antagonist that results in one or more of: (a) a reduction in the severity or duration of one or more symptoms of eosinophilic gastroenteritis (e.g., EoG and/or EoD); (b) a reduction in the number of eosinophils in a region of the gastrointestinal tract (e.g., stomach or small intestine); (c) an improvement in one or more anatomical, endoscopic, or histological features of the gastrointestinal tract (e.g., stomach or small intestine); (d) normalization of one or more EGE-associated biomarkers or gene expression signatures; and/or (e) a reduction in the use or need for concomitant or rescue treatment with another agent (e.g., reduced or eliminated use of systemic and/or swallowed topical corticosteroids).

In the case of an anti-IL-4R antibody, a therapeutically effective amount can be from about 0.05 mg to about 600 mg, about 50 mg to about 600 mg, about 50 mg to about 300 mg, or about 100 mg to about 300 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-IL-4R antibody. In some embodiments, a therapeutically effective amount is from about 50 mg to about 600 mg, or from about 100 mg to about 600 mg, or from about 50 mg to about 400 mg. In certain embodiments, 50 mg, 75 mg, 100 mg, 125 mg, 150 mg, 200 mg, 250 mg, or 300 mg of an anti-IL-4R antibody is administered to a subject.

The amount of IL-4R antagonist (e.g., anti-IL-4R antibody) contained within the individual doses may be expressed in terms of milligrams of antibody per kilogram of subject body weight (i.e., mg/kg). For example, the IL-4R antagonist may be administered to a subject at a dose of about 0.0001 to about 10 mg/kg of subject body weight, e.g., at a dose of about 1 mg/kg to about 10 mg/kg, at a dose of about 2 mg/kg to about 9 mg/kg, or at a dose of about 3 mg/kg to about 8 mg/kg. In some embodiments, the IL-4R antagonist may be administered to a subject at a dose of about 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg.

In some embodiments, the methods disclosed herein comprise administering an IL-4R antagonist to a subject at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In some embodiments, the methods disclosed herein comprise administering an IL-4R antagonist to a subject once weekly, once every two weeks, once every three weeks, or once every four weeks. In some embodiments, an IL-4R antagonist (e.g., an anti-IL-4R antibody as disclosed herein) is administered once weekly (QW), once every two weeks (Q2W), once every three weeks (Q3W), or once every four weeks (Q4W) in an amount of about 50 mg to about 600 mg, e.g., about 75 mg, 100 mg, 150 mg, 200 mg, or 300 mg.

In some embodiments, multiple doses of an IL-4R antagonist are administered to a subject over a defined time course. In some embodiments, the methods of the present disclosure comprise sequentially administering to a subject multiple doses of an IL-4R antagonist. As used herein, “sequentially administering” means that each dose of IL-4R antagonist is administered to the subject at a different point in time, e.g., on different days separated by a predetermined interval (e.g., hours, days, weeks, or months). In some embodiments, the methods of the disclosure comprise sequentially administering to the patient a single initial dose of an IL-4R antagonist, followed by one or more secondary doses of the IL-4R antagonist, and optionally followed by one or more tertiary doses of the IL-4R antagonist.

The terms “initial dose,” “secondary doses,” and “tertiary doses,” refer to the temporal sequence of administration of the IL-4R antagonist. Thus, the “initial dose” is the dose which is administered at the beginning of the treatment regimen (also referred to as the “loading dose”); the “secondary doses” are the doses which are administered after the initial dose; and the “tertiary doses” are the doses which are administered after the secondary doses. The initial, secondary, and tertiary doses may all contain the same amount of IL-4R antagonist, but generally may differ from one another in terms of frequency of administration. In certain embodiments, however, the amount of IL-4R antagonist contained in the initial, secondary, and/or tertiary doses varies from one another (e.g., adjusted up or down as appropriate) during the course of treatment. In some embodiments, one or more (e.g., 1, 2, 3, 4, or 5) doses are administered at the beginning of the treatment regimen as “loading doses” followed by subsequent doses that are administered on a less frequent basis (e.g., “maintenance doses”).

In some embodiments, the initial or loading dose and the one or more secondary or maintenance doses each contain the same amount of the IL-4R antagonist. In other embodiments, the initial dose comprises a first amount of the IL-4R antagonist, and the one or more secondary doses each comprise a second amount of the IL-4R antagonist. For example, the first amount of the IL-4R antagonist can be 1.5×, 2×, 2.5×, 3×, 3.5×, 4× or 5× or more than the second amount of the IL-4R antagonist. In some embodiments, one or more maintenance doses of the IL-4R antagonist are administered without a loading dose.

In some embodiments, a loading dose is a “split dose” that is administered as two or more doses (e.g., 2, 3, 4, or 5 doses) that are administered on separate days. In some embodiments, a loading dose is administered as a split dose wherein the two or more doses are administered at least about one week apart. In some embodiments, a loading dose is administered as a split dose wherein the two or more doses are administered about 1 week, 2 weeks, 3 weeks, or 4 weeks apart. In some embodiments, the loading dose is split evenly over the two or more doses (e.g., half of the loading dose is administered as the first portion and half of the loading dose is administered as the second portion). In some embodiments, the loading dose is split unevenly over the two or more doses (e.g., more than half of the loading dose is administered as the first portion and less than half of the loading dose is administered as the second portion).

In some embodiments, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of IL-4R antagonist which is administered to a patient prior to the administration of the very next dose in the sequence with no intervening doses.

The methods of the disclosure may comprise administering to a patient any number of secondary and/or tertiary doses of an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In some embodiments involving multiple secondary doses, each secondary dose is administered at the same frequency as the other secondary doses. For example, each secondary dose may be administered to the patient 1 week, 2 weeks, 3 weeks, or 4 weeks after the immediately preceding dose. Similarly, in some embodiments involving multiple tertiary doses, each tertiary dose is administered at the same frequency as the other tertiary doses. For example, each tertiary dose may be administered to the patient 1 week, 2 weeks, 3 weeks, or 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In some embodiments, for a subject having eosinophilic gastroenteritis (e.g., EoG and/or EoD) a therapeutically effective amount of an IL-4R antagonist (e.g., anti-IL-4R antibody) comprises 300 mg administered every week (QW). In some embodiments, no loading dose is administered. In some embodiments, a loading dose is administered, e.g., a loading dose of 600 mg.

In some embodiments, for a subject having eosinophilic gastroenteritis (e.g., EoG and/or EoD) a therapeutically effective amount of an IL-4R antagonist (e.g., anti-IL-4R antibody) comprises 300 mg administered every two weeks (Q2W). In some embodiments, no loading dose is administered. In some embodiments, a loading dose is administered, e.g., a loading dose of 600 mg.

### Therapeutic Dosage Forms

In another aspect, the present disclosure provides therapeutic dosage forms of an IL-4R antagonist (e.g., an anti-IL-4R antibody or antigen-binding fragment thereof) for use in treating a subject having eosinophilic gastroenteritis (e.g., EoG and/or EoD) as disclosed herein. In some embodiments, the IL-4R antagonist is an anti-IL-4R antibody or an antigen-binding fragment thereof having one or more CDR, HCVR, and/or LCVR sequences listed in Table 1. In some embodiments, the IL-4R antagonist is an anti-IL-4Rα antibody or antigen-binding fragment thereof that comprises the HCDRs of an HCVR comprising the amino acid sequence of SEQ ID NO:1 and the LCDRs of an LCVR comprising the amino acid sequence of SEQ ID NO:2 (e.g., dupilumab).

In some embodiments, the therapeutic dose is 300 mg of the IL-4R antagonist (e.g., anti-IL-4R antibody) and is for administration every week (QW).

In some embodiments, the therapeutic dose is 300 mg of the IL-4R antagonist (e.g., anti-IL-4R antibody) and is for administration every two weeks (Q2W).

### EGE-Related Parameters

In some embodiments, the therapeutic methods disclosed herein result in an improvement in one or more endpoints or EGE-related parameters that are used to assess the presence or severity of EGE (e.g., EoG with or without EoD) in a subject. Examples of EGE-related parameters include, but are not limited to: (a) change (e.g., reduction) in eosinophil count (e.g., gastric eosinophil count or duodenal eosinophil count); (b) change in the severity and/or extent of histologic features in the gastrointestinal tract (e.g., stomach or small intestine), e.g., as measured using the EoGHSS or EoDHSS; (c) change in one or more gastrointestinal characteristics, e.g., as measured using the EG-REFS or ED-REFS; (d) change (e.g., normalization) in the levels of one or more EGE-associated biomarkers or in an EGE gene expression signature; (e) change (e.g., reduction) in frequency and/or intensity of symptoms, e.g., as measured using the EoG/EoD Symptom Questionnaire (EoG/EoD-SQ), the Patient Global Impression of Change (PGI-C), the Patient Global Impression of Severity (PGI-S), the Clinical Global Impression of Change (CGI-C), the Clinical Global Impression of Severity (CGI-S), the EoG/EoD Quality of Life Questionnaire (EoG/EoD QoL), or the European Quality of Life 5-Dimension 5-Level Scale (EQ-5D-5L). Methods for assessing these and other EGE-related parameters are described in the Examples section below.

To determine whether an EGE-related parameter has “improved,” the parameter is quantified at baseline (e.g., prior to the start of treatment with the IL-4R antagonist) and at one or more timepoints after administration of the IL-4R antagonist. For example, an EGE-related parameter may be measured at day 1, day 2, day 3, day 4, day 5, day 6, day 7, day 8, day 9, day 10, day 11, day 12, day 14, day 15, day 22, day 25, day 29, day 36, day 43, day 50, day 57, day 64, day 71, day 85; or at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with a pharmaceutical composition of the present disclosure. The difference between the value of the parameter at a particular timepoint following initiation of treatment and the value of the parameter at baseline is used to establish whether there has been an improvement in the EGE-related parameter.

In some embodiments, treatment of a subject with an IL-4R antagonist (e.g., an anti-IL-4R antibody) results in an improvement (e.g., reduction) in peak gastric eosinophil count or peak duodenal eosinophil count. “Peak eosinophil count” refers to the number of eosinophils contained within one high power field (hpf).

In some embodiments, treatment with an IL-4R antagonist results in a decrease in peak gastric eosinophil count relative to baseline (e.g., a subject's peak count prior to the onset of treatment). In some embodiments, treatment with an IL-4R antagonist results in peak gastric eosinophil count of at least 10%, 15%, 20%, 25%, 30%, 35%, 40%, 45%, 50% or more relative to baseline. In some embodiments, treatment with an IL-4R antagonist results in a decrease in peak gastric eosinophil count to less than 30 eos/hpf, e.g., ≤20 eos/hpf, ≤15 eos/hpf, ≤10 eos/hpf, or ≤6 eos/hpf. In some embodiments, treatment with an IL-4R antagonist results in a decrease in peak gastric eosinophil count to ≤6 eos/hpf, ≤5 eos/hpf, ≤4 eos/hpf, ≤3 eos/hpf, ≤2 eos/hpf, or ≤1 eos/hpf. In some embodiments, treatment with an IL-4R antagonist results in histological disease remission. In some embodiments, the change in peak gastric eosinophil count is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

In some embodiments, treatment with an IL-4R antagonist results in a decrease in peak duodenal eosinophil count relative to baseline (e.g., a subject's peak count prior to the onset of treatment). In some embodiments, treatment with an IL-4R antagonist results in peak duodenal eosinophil count of at least 10%, 15%, 20%, 25%, 30%, 35%, 40%, 45%, 50% or more relative to baseline. In some embodiments, treatment with an IL-4R antagonist results in a decrease in peak duodenal eosinophil count to less than 30 eos/hpf, e.g., ≤20 eos/hpf, ≤15 eos/hpf, ≤10 eos/hpf, or ≤6 eos/hpf. In some embodiments, treatment with an IL-4R antagonist results in a decrease in peak duodenal eosinophil count to ≤6 eos/hpf, ≤5 eos/hpf, ≤4 eos/hpf, ≤3 eos/hpf, ≤2 eos/hpf, or ≤1 eos/hpf. In some embodiments, treatment with an IL-4R antagonist results in histological disease remission. In some embodiments, the change in peak duodenal eosinophil count is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

In some embodiments, treatment of a subject with an IL-4R antagonist (e.g., an anti-IL-4R antibody) results in an improvement in one or more histological features of EGE (e.g., EoG and/or EoD). In some embodiments, treatment of a subject with an IL-4R antagonist (e.g., an anti-IL-4R antibody) results in an improvement in EoG scores from the Histology Scoring System (EoGHSS). In some embodiments, treatment of a subject with an IL-4R antagonist (e.g., an anti-IL-4R antibody) results in an improvement in EoD scores from the Histology Scoring System (EoDHSS).

EoGHSS scores evaluate 11 features of gastric tissue: lamina propria eosinophils sheets, periglandular circumferential collars, eosinophils in surface epithelium, eosinophil glandulitis, eosinophil gland abscess, eosinophils in muscularis mucosa and submucosa, lamina propria fibroplasia, lamina propria smooth muscle hyperplasia, reactive epithelial changes, acute inflammation, and surface erosion/ulcer. In some embodiments, each feature is given a score from 0 to 3 (0=normal; 3=maximum change), and the total score is the sum of the features scores divided by the maximum possible score. In some embodiments, treatment with an IL-4R antagonist results in a decrease in EoGHSS score of at least 10%, 15%, 20%, 25%, 30%, 35%, 40%, 45%, 50% or more relative to baseline (e.g., a subject's EoGHSS score prior to the onset of treatment). In some embodiments, the change in EoGHSS score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

EoDHSS scores evaluate 11 features of duodenal tissue: lamina propria eosinophils sheets, pericryptal circumferential collars, eosinophils in surface epithelium, eosinophil cryptitis, subcryptal eosinophil aggregates, subcryptal lymphoplasmacytic inflammation, eosinophil crypt abscess, eosinophils in muscularis mucosa/submucosa, villous atrophy, elongated crypts, reactive epithelial changes, acute inflammatory cells, surface erosion/ulceration, and intraepithelial lymphocytosis. In some embodiments, each feature is given a score from 0 to 3 (0=normal; 3=maximum change), and the total score is the sum of the features scores divided by the maximum possible score. In some embodiments, treatment with an IL-4R antagonist results in a decrease in EoDHSS score of at least 10%, 15%, 20%, 25%, 30%, 35%, 40%, 45%, 50% or more relative to baseline (e.g., a subject's EoDHSS score prior to the onset of treatment). In some embodiments, the change in EoDHSS score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

In some embodiments, treatment of a subject with an IL-4R antagonist (e.g., an anti-IL-4R antibody) results in an improvement in one or more endoscopic features of EGE, e.g., inflammatory and remodeling features in the stomach or duodenum. In some embodiments, treatment of a subject with an IL-4R antagonist (e.g., an anti-IL-4R antibody) results in an improvement in EG-REFS score (for patients with EoG), ED-REFS (for patients with EoD), or EoE-EREFS score (for patients with comorbid EoE).

The EoG Endoscopic Reference Score (EG-REFS) is an endoscopic grading system that was specifically developed to evaluate eosinophilic gastritis and is completed by the endoscopist. It has been used in therapeutic trials and captures granularity (0-2 scale), erosion/ulceration (0-6), raised lesions (0-2), erythema (0-2), friability/bleeding (0-2), and folds (0-1) of the gastric fundus, body, and antrum. Pyloric stenosis (0-1) is also captured. The maximum EG-REFs total score is 46. In some embodiments, treatment with an IL-4R antagonist results in a decrease in EG-REFS score of at least 10%, 15%, 20%, 25%, 30%, 35%, 40%, 45%, 50% or more relative to baseline (e.g., a subject's EG-REFS score prior to the onset of treatment). In some embodiments, the change in EG-REFS score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist. In some embodiments, treatment with an IL-4R antagonist results in an improvement in one or more EG-REFS subscores.

The EoD Endoscopic Reference Score (ED-REFS) is an endoscopic grading system that was specifically developed to evaluate eosinophilic duodenitis and is completed by the endoscopist. It has been used in therapeutic trials and captures granularity (0-2 scale), erythema (0-2), friability/bleeding (0-2), ulceration (0-4), denuded patches (0-2), and stricture (0-2). The maximum ED-REFs total score is 14. In some embodiments, treatment with an IL-4R antagonist results in a decrease in EG-REFS score of at least 10%, 15%, 20%, 25%, 30%, 35%, 40%, 45%, 50% or more relative to baseline (e.g., a subject's ED-REFS score prior to the onset of treatment). In some embodiments, the change in ED-REFS score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist. In some embodiments, treatment with an IL-4R antagonist results in an improvement in one or more ED-REFS subscores.

The EoE-EREFS (edema, rings, exudates, furrows, strictures) is validated scoring system for inflammatory and remodeling features of disease that is used to measure endoscopically identified EoE esophageal mucosal inflammatory and remodeling features (Hirano, et al., Gut, 2013, 62:489-495). In some embodiments, treatment with an IL-4R antagonist results in a decrease in EoE-EREFS score of at least 10%, 15%, 20%, 25%, 30%, 35%, 40%, 45%, 50% or more relative to baseline (e.g., a subject's EoE-EREFS score prior to the onset of treatment). In some embodiments, the change in EoE-EREFS score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist. In some embodiments, treatment with an IL-4R antagonist results in an improvement in one or more EoE-EREFS subscores.

In some embodiments, treatment of a subject with an IL-4R antagonist (e.g., an anti-IL-4R antibody) results in a normalization of one or more EGE-associated biomarkers, an Eosinophilic Gastritis Diagnostic Panel (EGDP) gene signature, a Type 2 inflammation gene signature, and/or a Normalized Enrichment Score (NES) calculated for a set of EGE-associated genes (e.g., EoG-associated genes or EoD-associated genes). In some embodiments, treatment of a subject with an IL-4R antagonist suppresses an EGDP gene signature, a Type 2 inflammation gene signature, and/or an NES calculated for a set of EGE-associated or Type 2 inflammation genes. As used herein, the term “EGE-associated biomarker” refers to a biological response, cell type, parameter, protein, polypeptide, enzyme, enzyme activity, metabolite, nucleic acid, carbohydrate, or other biomolecule which is present or detectable in an EGE patient at a level or amount that is different from (e.g., greater than or less than) the level or amount of the marker present or detectable in a non-EGE patient. Exemplary EGE-associated biomarkers include, but are not limited to, e.g., gastric or duodenal eosinophils, cytokines/chemokines (CCL11, CCL18, CCL24, CCL26, IL13RA2, and IL5), eosinophilia (CLC), cell adhesion (CDH26), antimicrobial defense (KLK7, DEFB1), epithelial-related (MUC4), fibrosis (BMP3 and COL2A1), ion transportation (SLC26A7), neurosensory activity (GABRA1, GLDN, NPY, and TAC1), and stomach-related processes (ATP4A and SST). The term “EGE gene signature” refers to a differential gene expression profile of gastric biopsies of EGE patients (e.g., EoG with or without EoD) compared to healthy controls and is also referred to as an “EGE disease transcriptome” (Caldwell, et al., J Allergy Clin Med, 2014, 134:1114-1124). In some embodiments, the EGE gene signature is a differential gene expression profile associated with EoG, also referred to herein as an “EoG disease transcriptome” or an “EoG gene signature.” In some embodiments, an EGE gene signature is a smaller gene set, such as the EGDP panel (Shoda, et al., J Allergy Clin Med, 2020, 145:255-269). A “Type 2 inflammation gene signature” refers to a transcriptome for a set of genes associated with type 2 inflammation. Exemplary Type 2 inflammation-associated genes include, but are not limited to, CCL26, ALOX15, CCR3, and IL1RL1. An exemplary gene list for a Type 2 inflammation gene signature is shown in WO2021/237110, incorporated by reference herein. A Normalized Enrichment Score (NES) reflects the degree to which the activity level of a set of transcripts is overrepresented at the extremes (top or bottom) of the entire ranked list of transcripts within a sample and is normalized by accounting for the number of transcripts in the set (Subramanian, et al., Proc Natl Acad Sci USA, 2005, 102:15545-50) (Barbie, et al., Nature, 2009, 462:108-112).

In some embodiments, the EGE-associated biomarkers, EGE gene signature, Type 2 inflammation gene signature, and/or NES are determined using a tissue sample from the subject (e.g., gastric or duodenal biopsy samples). In some embodiments, treatment of a subject with an IL-4R antagonist results in a normalization of one or more EGE-associated biomarkers, an EGE gene signature, a Type 2 inflammation gene signature, and/or an NES, relative to baseline (e.g., a subject's level of expression of the EGE-associated biomarker(s), EGE gene signature, or NES prior to the onset of treatment), e.g., as measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist. In some embodiments, treatment of a subject with an IL-4R antagonist suppresses a NES for one or more EGE-associated biomarkers, an EGE gene signature, or a Type 2 inflammation gene signature, relative to baseline (e.g., a subject's NES prior to the onset of treatment), e.g., as measured at day 8, 15, 22, 25, 29, 36, 43, 50, 57, 64, 71, 85, 113 or later following administration of the IL-4R inhibitor, or after 2, 4, 6, 8, 10, 12, 14, 16, 18, 20, or 24 weeks of treatment with the IL-4R antagonist.

In some embodiments, treatment of a subject with an IL-4R antagonist (e.g., an anti-IL-4R antibody) results in an improvement in one or more other signs or symptoms of EGE (e.g., EoG and/or EoD) or in health-related quality of life.

In some embodiments, treatment results in an improvement in EoG/EoD Symptom Questionnaire score. The EoG/EoD-SQ is a novel PRO measure, designed to be collected daily, that has been developed to assess participant-reported symptoms of EoG (with or without EoD) among adults and adolescents. The EoG/EoD-SQ assesses 9 symptoms of EoG (with or without EoD): stomach pain, stomach cramping, nausea, heartburn, bloating, early satiety, loss of appetite, vomiting, and diarrhea. The severity of stomach pain, stomach cramping, nausea, heartburn, bloating, early satiety, and loss of appetite is assessed using an 11-point numerical rating scale (NRS; zero through 10); for vomiting and diarrhea the frequency of episodes are assessed. Higher scores indicate a higher symptom burden. Participant-reported severity scores for each symptom in the Total Symptom Score (TSS) (stomach pain, stomach cramping, nausea, bloating, early satiety, loss of appetite; each reported on a 0-10 scale) are summed on each day with eDiary data (maximum daily score of 60). The TSS is then calculated by averaging daily sum scores over all days with eDiary data in a 7-day period, with a maximum TSS of 60. Heartburn is excluded from the TSS as this symptom may be misinterpreted as esophageal dysfunction whereas other symptoms localize to the stomach/small intestine (site of eosinophilic inflammation in EoG [with or without EoD]). In some embodiments, prior to the start of treatment the subject has a baseline EoG/EoD-SQ TSS of 20, e.g., a baseline TSS of at least 25, 30, 35, or 40. In some embodiments, prior to the onset of treatment the subject has a baseline average severity score of 4 for at least 2 components of the EoG/EoD-SQ TSS (i.e., two or more of the components stomach pain, stomach cramping, nausea, bloating, early satiety, and loss of appetite). In some embodiments, the subject has a baseline average severity score of ≥4 for at least 2, at least 4, at least 5, or all 6 components of the EoG/EoD-SQ TSS. In some embodiments, the subject has a baseline average severity score of ≥5 or ≥6 for at least 2, at least 4, at least 5, or all 6 components of the EoG/EoD-SQ TSS. In some embodiments, treatment with an IL-4R antagonist results in a decrease in EoG/EoD-SQ score of at least 10%, 15%, 20%, 25%, 30%, 35%, 40%, 45%, 50% or more relative to baseline. In some embodiments, the change in EoG/EoD-SQ score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist. In some embodiments, treatment with an IL-4R antagonist results in a decrease in the number of days, or the number of total segments within a day (e.g., night, morning, afternoon, evening) with 1 or more EGE signs as measured by the EoG/EoD-SQ. In some embodiments, treatment with an IL-4R antagonist results in an improvement in one or more signs/symptoms measured by the EoG/EoD-SQ.

In some embodiments, treatment results in an improvement in PGI-C score. The Patient Global Impression of Change (PGI-C) is a 10-item PRO measure that assesses the participant's impression of the overall change (improvement or worsening) in their EoG (with or without EoD) symptoms and change in individual EoG/EoD symptoms since study treatment initiation, using a 7-level response scale (from “very much better” to “very much worse”). In some embodiments, treatment with an IL-4R antagonist results in a decrease in PGI-C score of at least 10%, 15%, 20%, 25%, 30%, 35%, 40%, 45%, 50% or more relative to baseline. In some embodiments, the change in PGi-C score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

In some embodiments, treatment results in an improvement in PGI-S score. The Patient Global Impression of Severity (PGI-S) is a 10-item PRO measure that assesses the participant's impression of the overall severity of their EoG (with or without EoD) symptoms and the severity of individual EoG/EoD symptoms over the past 7 days, using a 5-level response scale (from “none” to “very severe”). In some embodiments, treatment with an IL-4R antagonist results in a decrease in PGI-S score of at least 10%, 15%, 20%, 25%, 30%, 35%, 40%, 45%, 50% or more relative to baseline. In some embodiments, the change in PGI-S score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

In some embodiments, treatment results in an improvement in CGI-C score. The Clinician Global Impression of Change (CGI-C) is a single-item observer-reported outcome measure that assesses the study investigator's/clinician's impression of the overall change (improvement or worsening) in the participant's EoG (with or without EoD) since study treatment initiation, using a 7-level response scale (from “very much improved” to “very much worse”). In some embodiments, treatment with an IL-4R antagonist results in a decrease in CGI-C score of at least 10%, 15%, 20%, 25%, 30%, 35%, 40%, 45%, 50% or more relative to baseline. In some embodiments, the change in CGI-C score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

In some embodiments, treatment results in an improvement in CGI-S score. The Clinician Global Impression of Severity (CGI-S) is a single-item observer-reported outcome measure that assesses the study investigator's/clinician's impression of the overall severity of the participant's EoG (with or without EoD) based on their assessment of the participant on the day of questionnaire completion, using a 4-level response scale (from “mild” to “very severe”). In some embodiments, treatment with an IL-4R antagonist results in a decrease in CGI-S score of at least 10%, 15%, 20%, 25%, 30%, 35%, 40%, 45%, 50% or more relative to baseline. In some embodiments, the change in CGI-S score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

In some embodiments, treatment results in an improvement in EoG/EoD Quality of Life Questionnaire (EoG/EoD QoL) score. The EoG/EoD QoL is a disease-specific measure of health-related QoL in patients with EoG (with or without EoD) developed by the sponsor. The EoG/EoD QoL measures EoG (with or without EoD) impact on emotional, social, functional, work and school, and sleep aspects. Participants assess the degree of impact over the past 7 days using a 5-level scale (ranging from “Not at all” to “Extremely” or from “None of the time” to “All of the time” as appropriate). In some embodiments, treatment with an IL-4R antagonist results in a decrease in EoG/EoD QoL score of at least 10%, 15%, 20%, 25%, 30%, 35%, 40%, 45%, 50% or more relative to baseline. In some embodiments, the change in EoG/EoD QoL score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

In some embodiments, treatment results in an improvement in European Quality of Life 5-Dimension 5-Level scale (EQ-5D-5L) score. The EQ-5D-5L is a standardized questionnaire used to assess health status (Brooks, 1996) (Rabin, et al., Value Health, 2014, 17:70-76). It consists of a descriptive system and the EQ visual analogue scale (EQ VAS). The descriptive system comprises the following 5 dimensions: mobility, self-care, usual activities, pain/discomfort, and anxiety/depression. For each dimension, participants select one of 5 levels: no problems, slight problems, moderate problems, severe problems, and extreme problems. The EQ VAS records the participant's self-rated health on a vertical visual analogue scale where the endpoints are labelled ‘Best imaginable health state’ and ‘Worst imaginable health state’. In some embodiments, treatment with an IL-4R antagonist results in an improvement in EQ-5D-5L score of at least 10%, 15%, 20%, 25%, 30%, 35%, 40%, 45%, 50% or more relative to baseline. In some embodiments, the change in EQ-5D-5L score is measured at day 8, 15, 22, 25, 29, 36, 43, 50, 57, 64, 71, 85, 113 or later following administration of the IL-4R antagonist, or after 2, 4, 6, 8, 10, 12, 14, 16, 18, 20, or 24 weeks of treatment with the IL-4R antagonist.

### Combination Therapies

In some embodiments, the methods of the present disclosure comprise administering to the subject (e.g., a subject having EGE) an IL-4R antagonist according to the disclosure (e.g., an anti-IL-4R antibody) in combination with one or more additional therapeutic agents. As used herein, the expression “in combination with” means that the additional therapeutic agents are administered before, after, or concurrent with the pharmaceutical composition comprising the IL-4R antagonist. The term “in combination with” also includes sequential or concomitant administration of IL-4R antagonist and a second therapeutic agent or therapy.

For example, when administered “before” the pharmaceutical composition comprising the IL-4R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the pharmaceutical composition comprising the IL-4R antagonist. When administered “after” the pharmaceutical composition comprising the IL-4R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours or about 72 hours after the administration of the pharmaceutical composition comprising the IL-4R antagonist. Administration “concurrent” or with the pharmaceutical composition comprising the IL-4R antagonist means that the additional therapeutic agent is administered to the subject in a separate dosage form within less than about 10 minutes (before, after, or at the same time) of administration of the pharmaceutical composition comprising the IL-4R antagonist, or administered to the subject as a single combined dosage formulation comprising both the additional therapeutic agent and the IL-4R antagonist.

In some embodiments, the second therapeutic agent or therapy is an IL-1p inhibitor, an IL-5 or IL-5R inhibitor (e.g., an anti-IL-5 or anti-IL-5R antibody such as benralizumab, mepolizumab, or reslizumab), an IL-9 inhibitor, an IL-13 inhibitor (e.g., an anti-IL-13 antibody such as tralokinumab, RPC4046, or QAX576), an IL-17 inhibitor, an IL-25 inhibitor, a TNFα inhibitor (e.g., an anti-TNFα antibody such as infliximab or adalimumab), an eotaxin-3 inhibitor, an IgE inhibitor (e.g., an anti-IgE antibody such as omalizumab), a TSLP inhibitor (e.g., an anti-TSLP antibody such as tezepelumab), a CRTH2 inhibitor, a Siglec-8 inhibitor, a prostaglandin D2 inhibitor, an integrin inhibitor (e.g., an integrin α4β7 inhibitor such as vedolizumab), an eotaxin inhibitor, an immunosuppressant, a topical corticosteroid, an oral corticosteroid, a systemic corticosteroid, an inhaled corticosteroid, a glucocorticoid, a PPI, a decongestant, an antihistamine, a leukotriene inhibitor, a non-steroidal anti-inflammatory drug (NSAID), allergen removal, or diet management. In some embodiments, the IL-4R antagonist is used in combination with diet management. In some embodiments, the IL-4R antagonist is used in combination with a PPI, e.g., omeprazole, esomeprazole, lansoprazole, dexlansoprazole, rabeprazole, or pantoprazole. In some embodiments, the IL-4R antagonist is used in combination with a swallowed topical corticosteroid, e.g., budesonide or fluticasone. In some embodiments, the IL-4R antagonist is used in combination with a systemic corticosteroid, e.g., prednisone or prednisolone.

In some embodiments, administration of the IL-4R antagonist reduces dependence on or the need for using a concurrent therapy (e.g., a systemic corticosteroid or swallowed topical corticosteroid). In some embodiments, administration of the IL-4R antagonist in combination with the second therapy reduces the amount of the second therapy used by the patient by at least 20%, at least 30%, at least 40% or at least 50% as compared to the amount used by the subject before treatment with the IL-4R antagonist. In some embodiments, administration of the IL-4R antagonist eliminates the need for the second therapy.

In some embodiments, the methods of the present disclosure comprise administering to the subject (e.g., a subject having EGE, EoG, or EoD) a combination therapy comprising (i) an IL-4R antagonist according to the disclosure (e.g., an anti-IL-4R antibody), and (ii) a systemic corticosteroid or a swallowed topical corticosteroid. In some embodiments, the combination therapy comprises an IL-4R antagonist according to the disclosure (e.g., an anti-IL-4R antibody) and a systemic corticosteroid (e.g., prednisone or prednisolone). In some embodiments, the combination therapy comprises an IL-4R antagonist according to the disclosure (e.g., an anti-IL-4R antibody) and a swallowed topical corticosteroid (e.g., budesonide or fluticasone).

TABLE 1
Informal Sequence Listing

| SEQ | | |
|---|---|---|
| ID NO | Sequence | Description |
| 1 | EVQLVESGGGLEQPGGSLRLSCAGSGFTFRDYAMTWVRQAPGKGLEWVSSISGSG | Dupilumab HCVR amino |
| | GNTYYADSVKGRFTISRDNSKNTLYLQMNSLRAEDTAVYYCAKDRLSITIRPRYYGLD | acid sequence |
| | VWGQGTTVTVS | |
| 2 | DIVMTQSPLSLPVTPGEPASISCRSSQSLLYSIGYNYLDWYLQKSGQSPQLLIYLGSNR | Dupilumab LCVR amino |
| | ASGVPDRFSGSGSGTDFTLKISRVEAEDVGFYYCMQALQTPYTFGQGTKLEIK | acid sequence |
| 3 | GFTFRDYA | Dupilumab HCDR1 |
| | | amino acid sequence |
| 4 | ISGSGGNT | Dupilumab HCDR2 |
| | | amino acid sequence |
| 5 | AKDRLSITIRPRYYGLDV | Dupilumab HCDR3 |
| | | amino acid sequence |
| 6 | QSLLYSIGYNY | Dupilumab LCDR1 amino |
| | | acid sequence |
| | LGS | Dupilumab LCDR2 amino |
| | | acid sequence |
| 8 | MQALQTPYT | Dupilumab LCDR3 amino |
| | | acid sequence |
| 9 | EVQLVESGGGLEQPGGSLRLSCAGSGFTFRDYAMTWVRQAPGKGLEWVSSISGSG | Dupilumab heavy chain |
| | GNTYYADSVKGRFTISRDNSKNTLYLQMNSLRAEDTAVYYCAKDRLSITIRPRYYGLD | amino acid sequence |
| | VWGQGTTVTVSSASTKGPSVFPLAPCSRSTSESTAALGCLVKDYFPEPVTVSWNSGA | |
| | LTSGVHTFPAVLQSSGLYSLSSVVTVPSSSLGTKTYTCNVDHKPSNTKVDKRVESKYGP | |
| | PCPPCPAPEFLGGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSSIEKTISKA | |
| | KGQPREPQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSLGK | |
| 10 | DIVMTQSPLSLPVTPGEPASISCRSSQSLLYSIGYNYLDWYLQKSGQSPQLLIYLGSNR | Dupilumab light chain |
| | ASGVPDRFSGSGSGTDFTLKISRVEAEDVGFYYCMQALQTPYTFGQGTKLEIKRTVAA | amino acid sequence |
| | PSVFIFPPSDEQLKSGTASVVCLLNNFYPREAKVQWKVDNALQSGNSQESVTEQDSK | |
| | DSTYSLSSTLTLSKADYEKHKVYACEVTHQGLSSPVTKSFNRGEC | |
| 11 | MKVLQEPTCVSDYMSISTCEWKMNGPTNCSTELRLLYQLVFLLSEAHTCIPENNGGA | Human IL-4Rα |
| | GCVCHLLMDDVVSADNYTLDLWAGQQLLWKGSFKPSEHVKPRAPGNLTVHTNVS | |
| | DTLLLTWSNPYPPDNYLYNHLTYAVNIWSENDPADFRIYNVTYLEPSLRIAASTLKSGI | |
| | SYRARVRAWAQCYNTTWSEWSPSTKWHNSYREPFEQH | |
| 12 | EIVLTQSPGTLSLSPGERATLSCRASQSVSNSYLAWYQQKPGQAPRLLIFGASSRATGI | SCB-VL-39 |
| | PDRFSGSGSGTDFTLTISRLEPEDFAVYYCQQYGSSPPWTFGQGTKVEIK | |
| 13 | EIVLTQSPGTLSLSPGERATLSCRASQSVSSSYLAWYQQKPGQAPRLLIYGASSRATGI | SCB-VL-40 |
| | PDRFSGSGSGTDFTLTISRLEPEDFAVYYCQQYGSSPPWTFGQGTKVEIK | |
| 14 | EIVLTQSPGTLSLSPGERATLSCRASQSVSSSYLAWYQQKPGQAPRLLIFGASSRAPGI | SCB-VL-41 |
| | PDRFSGSGSGTDFTLTISRLEPEDFAVYYCQQYGSSPPWTFGQGTKVEIK | |
| 15 | EIVLTQSPGTLSLSPGERATLSCRASQSVSNSYLAWYQQKPGQAPRLLIYGASSRATGI | SCB-VL-42 |
| | PDRFSGSGSGTDFTLTISRLEPEDFAVYYCQQYGSSPPWTFGQGTKVEIK | |
| 16 | EIVLTQSPGTLSLSPGERATLSCRASQSVSNSYLAWYQQKPGQAPRLLIFGASSRAPGI | SCB-VL-43 |
| | PDRFSGSGSGTDFTLTISRLEPEDFAVYYCQQYGSSPPWTFGQGTKVEIK | |
| 17 | EIVLTQSPGTLSLSPGERATLSCRASQSVSSSYLAWYQQKPGQAPRLLIYGASSRAPGI | SCB-VL-44 |
| | PDRFSGSGSGTDFTLTISRLEPEDFAVYYCQQYGSSPPWTFGQGTKVEIK | |
| 18 | EIVLTQSPGTLSLSPGERATLSCRASQSVSSSYLAWYQQKPGQAPRLLIFGASSRATGI | SCB-VL-45 |
| | PDRFSGSGSGTDFTLTISRLEPEDFAVYYCQQYDHSPPWTFGQGTKVEIK | |
| 19 | EIVLTQSPGTLSLSPGERATLSCRASQSVSSSYLAWYQQKPGQAPRLLIFGASSRATGI | SCB-VL-46 |
| | PDRFSGSGSGTDFTLTISRLEPEDFAVYYCQQYGSSAGWTFGQGTKVEIK | |
| 20 | EIVLTQSPGTLSLSPGERATLSCRASQSVSSSYLAWYQQKPGQAPRLLIFGASSRATGI | SCB-VL-47 |
| | PDRFSGSGSGTDFTLTISRLEPEDFAVYYCQQYDHSAGWTFGQGTKVEIK | |
| 21 | EIVLTQSPGTLSLSPGERATLSCRASQSVSNSYLAWYQQKPGQAPRLLIFGASSRATGI | SCB-VL-48 |
| | PDRFSGSGSGTDFTLTISRLEPEDFAVYYCQQYDHSPPWTFGQGTKVEIK | |
| 22 | EIVLTQSPGTLSLSPGERATLSCRASQSVSSSYLAWYQQKPGQAPRLLIYGASSRATGI | SCB-VL-49 |
| | PDRFSGSGSGTDFTLTISRLEPEDFAVYYCQQYDHSPPWTFGQGTKVEIK | |
| 23 | EIVLTQSPGTLSLSPGERATLSCRASQSVSSSYLAWYQQKPGQAPRLLIFGASSRAPGI | SCB-VL-50 |
| | PDRFSGSGSGTDFTLTISRLEPEDFAVYYCQQYDHSPPWTFGQGTKVEIK | |
| 24 | EIVLTQSPGTLSLSPGERATLSCRASQSVSSSYLAWYQQKPGQAPRLLIYGASSRAPGI | SCB-VL-51 |
| | PDRFSGSGSGTDFTLTISRLEPEDFAVYYCQQYDHSAGWTFGQGTKVEIK | |
| 25 | EIVLTQSPGTLSLSPGERATLSCRASQSVSNSYLAWYQQKPGQAPRLLIFGASSRAPGI | SCB-VL-52 |
| | PDRFSGSGSGTDFTLTISRLEPEDFAVYYCQQYDHSAGWTFGQGTKVEIK | |
| 26 | EIVLTQSPGTLSLSPGERATLSCRASQSVSNSYLAWYQQKPGQAPRLLIYGASSRATGI | SCB-VL-53 |
| | PDRFSGSGSGTDFTLTISRLEPEDFAVYYCQQYDHSAGWTFGQGTKVEIK | |
| 27 | EIVLTQSPGTLSLSPGERATLSCRASQSVSSSYLAWYQQKPGQAPRLLIFGASSRAPGI | SCB-VL-54 |
| | PDRFSGSGSGTDFTLTISRLEPEDFAVYYCQQYDHSAGWTFGQGTKVEIK | |
| 28 | EIVLTQSPGTLSLSPGERATLSCRASQSVSSSYLAWYQQKPGQAPRLLIYGASSRATGI | SCB-VL-55 |
| | PDRFSGSGSGTDFTLTISRLEPEDFAVYYCQQYDHSAGWTFGQGTKVEIK | |
| 29 | EIVLTQSPGTLSLSPGERATLSCRASQSVSNSYLAWYQQKPGQAPRLLIFGASSRATGI | SCB-VL-56 |
| | PDRFSGSGSGTDFTLTISRLEPEDFAVYYCQQYDHSAGWTFGQGTKVEIK | |
| 30 | EIVLTQSPGTLSLSPGERATLSCRASQSVSSSYLAWYQQKPGQAPRLLIFGASSRATGI | SCB-VL-57 |
| | PDRFSGSGSGTDFTLTISRLEPEDFAVYYCQQYGSSPPWTFGQGTKVEIK | |
| 31 | EIVLTQSPGT1LSLSPGERATLSCRASQSVSNSYLAWYQQKPGQAPRLLIYGASSRAPGI | SCB-VL-58 |
| | PDRFSGSGSGTDFTLTISRLEPEDFAVYYCQQYDHSAGWTFGQGTKVEIK | |
| 32 | EVQLVESGGGLVHPGGSLRLSCAGSGFTFSRNAMFWVRQAPGKGLEWVSGIGTGG | SCB-VH-59 |
| | ATNYADSVKGRFTISRDNAKNSLYLQMNSLRAEDMAVYYCARGRYYFDYWGQGTL | |
| | VTVSS | |
| 33 | EVQLVQSGGGLVQPGGSLRLSCAGSGFTFSRNAMFWVRQAPGKGLEWVSGIGTG | SCB-VH-60 |
| | GATNYADSVKGRFTISRDNAKNSLYLQMNSLRAEDMAVYYCARGRYYFDYWGQGT | |
| | LVTVSS | |
| 34 | EVQLVQSGGGLVHPGGSLRLSCAASGFTFSRNAMFWVRQAPGKGLEWVSGIGTGG | SCB-VH-61 |
| | ATNYADSVKGRFTISRDNAKNSLYLQMNSLRAEDMAVYYCARGRYYFDYWGQGTL | |
| | VTVSS | |
| 35 | EVQLVQSGGGLVHPGGSLRLSCAGSGFTFSRNAMFWVRQAPGKGLEWVSGIGTGG | SCB-VH-62 |
| | ATSYADSVKGRFTISRDNAKNSLYLQMNSLRAEDMAVYYCARGRYYFDYWGQGTLV | |
| | TVSS | |
| 36 | EVQLVQSGGGLVHPGGSLRLSCAGSGFTFSRNAMFWVRQAPGKGLEWVSGIGTGG | SCB-VH-63 |
| | ATNYADSVKGRFTISRDNAKNSLYLQMNSLRAEDTAVYYCARGRYYFDYWGQGTLV | |
| | TVSS | |
| 37 | EVQLVESGGGLVQPGGSLRLSCAGSGFTFSRNAMFWVRQAPGKGLEWVSGIGTGG | SCB-VH-64 |
| | ATNYADSVKGRFTISRDNAKNSLYLQMNSLRAEDMAVYYCARGRYYFDYWGQGTL | |
| | VTVSS | |
| 38 | EVQLVESGGGLVHPGGSLRLSCAASGFTFSRNAMFWVRQAPGKGLEWVSGIGTGG | SCB-VH-65 |
| | ATNYADSVKGRFTISRDNAKNSLYLQMNSLRAEDMAVYYCARGRYYFDYWGQGTL | |
| | VTVSS | |
| 39 | EVQLVQSGGGLVQPGGSLRLSCAASGFTFSRNAMFWVRQAPGKGLEWVSGIGTGG | SCB-VH-66 |
| | ATNYADSVKGRFTISRDNAKNSLYLQMNSLRAEDMAVYYCARGRYYFDYWGQGTL | |
| | VTVSS | |
| 40 | EVQLVQSGGGLVHPGGSLRLSCAGSGFTFSRNAMFWVRQAPGKGLEWVSGIGTGG | SCB-VH-67 |
| | ATSYADSVKGRFTISRDNAKNSLYLQMNSLRAEDTAVYYCARGRYYFDYWGQGTLV | |
| | TVSS | |
| 41 | EVQLVQSGGGLVHPGGSLRLSCAGSGFTFSRNAMFWVRQAPGKGLEWVSGIGTGG | SCB-VH-68 |
| | ATNYADSVKGRFTISRDNAKNSLYLQMNSLRAEDMAVYYCARGRYYFPWWGQGTL | |
| | VTVSS | |
| 42 | EVQLVESGGGLVHPGGSLRLSCAGSGFTFSRNAMFWVRQAPGKGLEWVSGIGTGG | SCB-VH-69 |
| | ATNYADSVKGRFTISRDNAKNSLYLQMNSLRAEDMAVYYCARGRYYFPWWGQGTL | |
| | VTVSS | |
| 43 | EVQLVQSGGGLVQPGGSLRLSCAGSGFTFSRNAMFWVRQAPGKGLEWVSGIGTG | SCB-VH-70 |
| | GATNYADSVKGRFTISRDNAKNSLYLQMNSLRAEDMAVYYCARGRYYFPWWGQG | |
| | TLVTVSS | |
| 44 | EVQLVQSGGGLVHPGGSLRLSCAASGFTFSRNAMFWVRQAPGKGLEWVSGIGTGG | SCB-VH-71 |
| | ATNYADSVKGRFTISRDNAKNSLYLQMNSLRAEDMAVYYCARGRYYFPWWGQGTL | |
| | VTVSS | |
| 45 | EVQLVQSGGGLVHPGGSLRLSCAGSGFTFSRNAMFWVRQAPGKGLEWVSGIGTGG | SCB-VH-72 |
| | ATSYADSVKGRFTISRDNAKNSLYLQMNSLRAEDMAVYYCARGRYYFPWWGQGTL | |
| | VTVSS | |
| 46 | EVQLVQSGGGLVHPGGSLRLSCAGSGFTFSRNAMFWVRQAPGKGLEWVSGIGTGG | SCB-VH-73 |
| | ATNYADSVKGRFTISRDNAKNSLYLQMNSLRAEDTAVYYCARGRYYFPWWGQGTL | |
| | VTVSS | |
| 47 | EVQLVQSGGGLVHPGRSLRLSCAGSGFTFSRNAMFWVRQAPGKGLEWVSGIGTGG | SCB-VH-74 |
| | ATNYADSVKGRFTISRDNAKNSLYLQMNSLRAEDMAVYYCARGRYYFDYWGQGTL | |
| | VTVSS | |
| 48 | EVQLVQSGGGLVHPGGSLRLTCAGSGFTFSRNAMFWVRQAPGKGLEWVSGIGTG | SCB-VH-75 |
| | GATNYADSVKGRFTISRDNAKNSLYLQMNSLRAEDMAVYYCARGRYYFDYWGQGT | |
| | LVTVSS | |
| 49 | EVQLVQSGGGLVHPGGSLRLSCAGSGFTFSRNAMHWVRQAPGKGLEWVSGIGTG | SCB-VH-76 |
| | GATNYADSVKGRFTISRDNAKNSLYLQMNSLRAEDMAVYYCARGRYYFDYWGQGT | |
| | LVTVSS | |
| 50 | EVQLVQSGGGLVHPGGSLRLSCAGSGFTFSRNAMFWVRQAPGEGLEWVSGIGTGG | SCB-VH-77 |
| | ATNYADSVKGRFTISRDNAKNSLYLQMNSLRAEDMAVYYCARGRYYFDYWGQGTL | |
| | VTVSS | |
| 51 | EVQLVQSGGGLVHPGGSLRLSCAGSGFTFSRNAMFWVRQAPGKGLEWVSGIGTGG | SCB-VH-78 |
| | ATNYADSVKGRFTISRDEAKNSLYLQMNSLRAEDMAVYYCARGRYYFDYWGQGTLV | |
| | TVSS | |
| 52 | EVQLVQSGGGLVHPGGSLRLSCAGSGFTFSRNAMFWVRQAPGKGLEWVSGIGTGG | SCB-VH-79 |
| | ATNYADSVKGRFTISRDNAKNSLYLQMNSLRAGDMAVYYCARGRYYFDYWGQGTL | |
| | VTVSS | |
| 53 | EVQLVQSGGGLVHPGGSLRLSCAGSGFTFDDYAMFWVRQAPGKGLEWVSGIGTG | SCB-VH-80 |
| | GATNYADSVKGRFTISRDNAKNSLYLQMNSLRAEDMAVYYCARGRYYFDYWGQGT | |
| | LVTVSS | |
| 54 | EVQLVQSGGGLVQPGGSLRLSCAASGFTFSRNAMFWVRQAPGKGLEWVSGIGTGG | SCB-VH-81 |
| | ATSYADSVKGRFTISRDNAKNSLYLQMNSLRAEDTAVYYCARGRYYFPWWGQGTLV | |
| | TVSS | |
| 55 | EVQLVESGGGLVHPGGSLRLSCAASGFTFSRNAMFWVRQAPGKGLEWVSGIGTGG | SCB-VH-82 |
| | ATSYADSVKGRFTISRDNAKNSLYLQMNSLRAEDTAVYYCARGRYYFPWWGQGTLV | |
| | TVSS | |
| 56 | EVQLVESGGGLVQPGGSLRLSCAGSGFTFSRNAMFWVRQAPGKGLEWVSGIGTGG | SCB-VH-83 |
| | ATSYADSVKGRFTISRDNAKNSLYLQMNSLRAEDTAVYYCARGRYYFPWWGQGTLV | |
| | TVSS | |
| 57 | EVQLVESGGGLVQPGGSLRLSCAASGFTFSRNAMFWVRQAPGKGLEWVSGIGTGG | SCB-VH-84 |
| | ATNYADSVKGRFTISRDNAKNSLYLQMNSLRAEDTAVYYCARGRYYFPWWGQGTL | |
| | VTVSS | |
| 58 | EVQLVESGGGLVQPGGSLRLSCAASGFTFSRNAMFWVRQAPGKGLEWVSGIGTGG | SCB-VH-85 |
| | ATSYADSVKGRFTISRDNAKNSLYLQMNSLRAEDMAVYYCARGRYYFPWWGQGTL | |
| | VTVSS | |
| 59 | EVQLVQSGGGLVHPGGSLRLSCAASGFTFSRNAMFWVRQAPGKGLEWVSGIGTGG | SCB-VH-86 |
| | ATSYADSVKGRFTISRDNAKNSLYLQMNSLRAEDTAVYYCARGRYYFPWWGQGTLV | |
| | TVSS | |
| 60 | EVQLVQSGGGLVQPGGSLRLSCAGSGFTFSRNAMFWVRQAPGKGLEWVSGIGTG | SCB-VH-87 |
| | GATSYADSVKGRFTISRDNAKNSLYLQMNSLRAEDTAVYYCARGRYYFPWWGQGTL | |
| | VTVSS | |
| 61 | EVQLVESGGGLVHPGGSLRLSCAGSGFTFSRNAMFWVRQAPGKGLEWVSGIGTGG | SCB-VH-88 |
| | ATSYADSVKGRFTISRDNAKNSLYLQMNSLRAEDTAVYYCARGRYYFPWWGQGTLV | |
| | TVSS | |
| 62 | EVQLVQSGGGLVHPGGSLRLSCAGSGFTFSRNAMFWVRQAPGKGLEWVSGIGTGG | SCB-VH-89 |
| | ATSYADSVKGRFTISRDNAKNSLYLQMNSLRAEDTAVYYCARGRYYFPWWGQGTLV | |
| | TVSS | |
| 63 | EVQLVESGGGLVQPGGSLRLSCAASGFTFSRNAMFWVRQAPGKGLEWVSGIGTGG | SCB-VH-90 |
| | ATNYADSVKGRFTISRDNAKNSLYLQMNSLRAEDMAVYYCARGRYYFPWWGQGTL | |
| | VTVSS | |
| 64 | EVQLVESGGGLVQPGGSLRLSCAASGFTFSRNAMFWVRQAPGKGLEWVSGIGTGG | SCB-VH-91 |
| | ATSYADSVKGRFTISRDNAKNSLYLQMNSLRAEDTAVYYCARGRYYFDYWGQGTLV | |
| | TVSS | |
| 65 | EVQLVQSGGGLVHPGGSLRLSCAGSGFTFSRNAMFWVRQAPGKGLEWVSGIGTGG | SCB-VH-92 |
| | ATNYADSVKGRFTISRDNAKNSLYLQMNSLRAEDMAVYYCARGRYYFDYWGQGTL | |
| | VTVSS | |
| 66 | EVQLVESGGGLVQPGGSLRLSCAASGFTFSRNAMFWVRQAPGKGLEWVSGIGTGG | SCB-VH-93 |
| | ATSYADSVKGRFTISRDNAKNSLYLQMNSLRAEDTAVYYCARGRYYFPWWGQGTLV | |
| | TVSS | |
| 67 | QVQLVQSGAEVKKPGASVKVSCKASGYAFTSYYMHWARQAPGQGLEWMGIINPS | MEDI-1-VH |
| | GGSTSYAQKFQGRVTMTRDTSTSTVYMELSSLRSEDTAVYYCARGKWWLDYWGKG | |
| | TLVTVSS | |
| 68 | QSVLTQPPSVSAAPGQKVTISCSGGSSNIGNSYVSWYQQLPGTAPKLLIYDNNKRPS | MEDI-1-VL |
| | GIPDRFSGSKSGTSATLAITGLQTGDEADYYCGTWDTSLSANYVFGTGTKLTVL | |
| 69 | QVQLVQSGAEVKKPGASVKVSCKASGYAFTSYYMHWARQAPGQGLEWMGIINPS | MEDI-2-VH |
| | GGSTSYAQKFQGRVTMTRDTSTSTVYMELSSLRSEDTAVYYCARGKWWLYNWGKG | |
| | TLVTVSS | |
| 70 | QSVLTQPPSVSAAPGQKVTISCSGGSSNIGNSYVSWYQQLPGTAPKLLIYDNNKRPS | MEDI-2-VL |
| | GIPDRFSGSKSGTSATLAITGLQTGDEADYYCGTWDTSQPPNPLFGTGTKLTVL | |
| 71 | QVQLVQSGAEVKKPGASVKVSCKASGYAFTSYYMHWARQAPGQGLEWMGIINPS | MEDI-3-VH |
| | GGSTSYAQKFQGRVTMTRDTSTSTVYMELSSLRSEDTAVYYCARGKLLKNPWGKGT | |
| | LVTVSS | |
| 72 | QSVLTQPPSVSAAPGQKVTISCSGGSSNIGNSYVSWYQQLPGTAPKLLIYDNNKRPS | MEDI-3-VL |
| | GIPDRFSGSKSGTSATLAITGLQTGDEADYYCGTWFGTPASNYVFGTGTKLTVL | |
| 73 | QVQLVQSGAEVKKPGASVKVSCKASGYAFTSYYMHWARQAPGQGLEWMGIINPS | MEDI-4-VH |
| | GGSTSYAQKFQGRVTMTRDTSTSTVYMELSSLRSEDTAVYYCARGKWWLYNWGKG | |
| | TLVTVSS | |
| 74 | QSVLTQPPSVSAAPGQKVTISCSGGSSNIGNSYVSWYQQLPGTAPKLLIYDNNKRPS | MEDI-4-VL |
| | GIPDRFSGSKSGTSATLAITGLQTGDEADYYCGTWDTSSPPQPIFGTGTKLTVL | |
| 75 | QVQLVQSGAEVKKPGASVKVSCKASGYAFTSYYMHWARQAPGQGLEWMGIINPS | MEDI-5-VH |
| | GGSTSYAQKFQGRVTMTRDTSTSTVYMELSSLRSEDTAVYYCARGKWWLYDWGKG | |
| | TLVTVSS | |
| 76 | QSVLTQPPSVSAAPGQKVTISCSGGSSNIGNSYVSWYQQLPGTAPKLLIYDNNKRPS | MEDI-5-VL |
| | GIPDRFSGSKSGTSATLAITGLQTGDEADYYCGTWDTSSPPQPIFGTGTKLTVL | |
| 77 | QVQLVQSGAEVKKPGASVKVSCKASGYAFTSYYMHWARQAPGQGLEWMGIINPS | MEDI-6-VH |
| | GGSTSYAQKFQGRVTMTRDTSTSTVYMELSSLRSEDTAVYYCARGKYWMYDWGKG | |
| | TLVTVSS | |
| 78 | QSVLTQPPSVSAAPGQKVTISCSGGSSNIGNSYVSWYQQLPGTAPKLLIYDNNKRPS | MEDI-6-VL |
| | GIPDRFSGSKSGTSATLAITGLQTGDEADYYCGTWDTSTTYHPIFGTGTKLTVL | |
| 79 | QVQLVQSGAEVKKPGASVKVSCKASGYAFTSYYMHWARQAPGQGLEWMGIINPS | MEDI-7-VH |
| | GGSTSYAQKFQGRVTMTRDTSTSTVYMELSSLRSEDTAVYYCARGKWWWQYWGK | |
| | GTLVTVSS | |
| 80 | QSVLTQPPSVSAAPGQKVTISCSGGSSNIGNSYVSWYQQLPGTAPKLLIYDNNKRPS | MEDI-7-VL |
| | GIPDRFSGSKSGTSATLAITGLQTGDEADYYCGTWDTSSPPQPIFGTGTKLTVL | |
| 81 | QVQLVQSGAEVKKPGASVKVSCKASGYAFTSYYMHWARQAPGQGLEWMGIINPS | MEDI-8-VH |
| | GGSTSYAQKFQGRVTMTRDTSTSTVYMELSSLRSEDTAVYYCARGKWWWQYWGK | |
| | GTLVTVSS | |
| 82 | QSVLTQPPSVSAAPGQKVTISCSGGSSNIGNSYVSWYQQLPGTAPKLLIYDNNKRPS | MEDI-8-VL |
| | GIPDRFSGSKSGTSATLAITGLQTGDEADYYCGTWDTSTTYHPIFGTGTKLTVL | |
| 83 | QVQLVQSGAEVKKPGASVKVSCKASGYAFTSYYMHWARQAPGQGLEWMGIINPS | MEDI-9-VH |
| | GGSTSYAQKFQGRVTMTRDTSTSTVYMELSSLRSEDTAVYYCARGKWWLYNWGKG | |
| | TLVTVSS | |
| 84 | QSVLTQPPSVSAAPGQKVTISCSGGSSNIGNSYVSWYQQLPGTAPKLLIYDNNKRPS | MEDI-9-VL |
| | GIPDRFSGSKSGTSATLAITGLQTGDEADYYCGTWDTSTTMYPLFGTGTKLTVL | |
| 85 | QVQLVQSGAEVKKPGASVKVSCKASGYAFTSYYMHWARQAPGQGLEWMGIINPS | MEDI-10-VH |
| | GGSTSYAQKFQGRVTMTRDTSTSTVYMELSSLRSEDTAVYYCARGKWWLYDWGKG | |
| | TLVTVSS | |
| 86 | QSVLTQPPSVSAAPGQKVTISCSGGSSNIGNSYVSWYQQLPGTAPKLLIYDNNKRPS | MEDI-10-VL |
| | GIPDRFSGSKSGTSATLAITGLQTGDEADYYCGTWDTSTVLTPIFGTGTKLTVL | |
| 87 | QVQLVQSGAEVKKPGASVKVSCKASGYAFTSYYMHWARQAPGQGLEWMGIINPS | MEDI-11-VH |
| | GGSTSYAQKFQGRVTMTRDTSTSTVYMELSSLRSEDTAVYYCARGKWWFYDWGKG | |
| | TLVTVSS | |
| 88 | QSVLTQPPSVSAAPGQKVTISCSGGSSNIGNSYVSWYQQLPGTAPKLLIYDNNKRPS | MEDI-11-VL |
| | GIPDRFSGSKSGTSATLAITGLQTGDEADYYCGTWDTSPSMIPLFGTGTKLTVL | |
| 89 | QVQLVQSGAEVKKPGASVKVSCKASGYAFTSYYMHWARQAPGQGLEWMGIINPS | MEDI-12-VH |
| | GGSTSYAQKFQGRVTMTRDTSTSTVYMELSSLRSEDTAVYYCARGKWWFYDWGKG | |
| | TLVTVSS | |
| 90 | QSVLTQPPSVSAAPGQKVTISCSGGSSNIGNSYVSWYQQLPGTAPKLLIYDNNKRPS | MEDI-12-VL |
| | GIPDRFSGSKSGTSATLAITGLQTGDEADYYCGTWDTSTTMYPLFGTGTKLTVL | |
| 91 | QVQLVQSGAEVKKPGASVKVSCKASGYAFTSYYMHWARQAPGQGLEWMGIINPS | MEDI-13-VH |
| | GGSTSYAQKFQGRVTMTRDTSTSTVYMELSSLRSEDTAVYYCARGKWWLYDWGKG | |
| | TLVTVSS | |
| 92 | QSVLTQPPSVSAAPGQKVTISCSGGSSNIGNSYVSWYQQLPGTAPKLLIYDNNKRPS | MEDI-13-VL |
| | GIPDRFSGSKSGTSATLAITGLQTGDEADYYCGTWDTSTTLQPLFGTGTKLTVL | |
| 93 | QVQLVQSGAEVKKPGASVKVSCKASGYAFTSYYMHWARQAPGQGLEWMGIINPS | MEDI-14-VH |
| | GGSTSYAQKFQGRVTMTRDTSTSTVYMELSSLRSEDTAVYYCARGKWWLYNWGKG | |
| | TLVTVSS | |
| 94 | QSVLTQPPSVSAAPGQKVTISCSGGSSNIGNSYVSWYQQLPGTAPKLLIYDNNKRPS | MEDI-14-VL |
| | GIPDRFSGSKSGTSATLAITGLQTGDEADYYCGTWDTSPPTKPLFGTGTKLTVL | |
| 95 | QVQLVQSGAEVKKPGASVKVSCKASGYAFTSYYMHWARQAPGQGLEWMGIINPS | MEDI-15-VH |
| | GGSTSYAQKFQGRVTMTRDTSTSTVYMELSSLRSEDTAVYYCARGKWWLYNWGKG | |
| | TLVTVSS | |
| 96 | QSVLTQPPSVSAAPGQKVTISCSGGSSNIGNSYVSWYQQLPGTAPKLLIYDNNKRPS | MEDI-15-VL |
| | GIPDRFSGSKSGTSATLAITGLQTGDEADYYCGTWDTSTHRHPLFGTGTKLTVL | |
| 97 | QVQLVQSGAEVKKPGASVKVSCKASGYAFTSYYMHWARQAPGQGLEWMGIINPS | MEDI-16-VH |
| | GGSTSYAQKFQGRVTMTRDTSTSTVYMELSSLRSEDTAVYYCARGKWWLYNWGKG | |
| | TLVTVSS | |
| 98 | QSVLTQPPSVSAAPGQKVTISCSGGSSNIGNSYVSWYQQLPGTAPKLLIYDNNKRPS | MEDI-16-VL |
| | GIPDRFSGSKSGTSATLAITGLQTGDEADYYCGTWDTSTTYHPIFGTGTKLTVL | |
| 99 | QVQLVQSGAEVKKPGASVKVSCKASGYAFTSYYMHWARQAPGQGLEWMGIINPS | MEDI-17-VH |
| | GGSTSYAQKFQGRVTMTRDTSTSTVYMELSSLRSEDTAVYYCARGKWWWQHWGK | |
| | GTLVTVSS | |
| 100 | QSVLTQPPSVSAAPGQKVTISCSGGSSNIGNSYVSWYQQLPGTAPKLLIYDNNKRPS | MEDI-17-VL |
| | GIPDRFSGSKSGTSATLAITGLQTGDEADYYCGTWDTSPVDRPIFGTGTKLTVL | |
| 101 | QVQLVQSGAEVKKPGASVKVSCKASGYAFTSYYMHWARQAPGQGLEWMGIINPS | MEDI-18-VH |
| | GGSTSYAQKFQGRVTMTRDTSTSTVYMELSSLRSEDTAVYYCARGKWWWQHWGK | |
| | GTLVTVSS | |
| 102 | QSVLTQPPSVSAAPGQKVTISCSGGSSNIGNSYVSWYQQLPGTAPKLLIYDNNKRPS | MEDI-18-VL |
| | GIPDRFSGSKSGTSATLAITGLQTGDEADYYCGTWDTSTTPMPVFGTGTKLTVL | |
| 103 | QVQLVQSGAEVKKPGASVKVSCKASGYAFTSYYMHWARQAPGQGLEWMGIINPS | MEDI-19-VH |
| | GGSTSYAQKFQGRVTMTRDTSTSTVYMELSSLRSEDTAVYYCARGKWWWQHWGK | |
| | GTLVTVSS | |
| 104 | QSVLTQPPSVSAAPGQKVTISCSGGSSNIGNSYVSWYQQLPGTAPKLLIYDNNKRPS | MEDI-19-VL |
| | GIPDRFSGSKSGTSATLAITGLQTGDEADYYCGTWDTSTTYHPIFGTGTKLTVL | |
| 105 | QVQLVQSGAEVKKPGASVKVSCKASGYAFTSYYMHWARQAPGQGLEWMGIINPS | MEDI-20-VH |
| | GGSTSYAQKFQGRVTMTRDTSTSTVYMELSSLRSEDTAVYYCARGKYWMYDWGKG | |
| | TLVTVSS | |
| 106 | QSVLTQPPSVSAAPGQKVTISCSGGSSNIGNSYVSWYQQLPGTAPKLLIYDNNKRPS | MEDI-20-VL |
| | GIPDRFSGSKSGTSATLAITGLQTGDEADYYCGTWDTSTVWEWPFGTGTKLTVL | |
| 107 | QVQLVQSGAEVKKPGASVKVSCKASGYAFTSYYMHWARQAPGQGLEWMGIINPS | MEDI-21-VH |
| | GGSASYAQKFQGRVTMTRDTSTSTVYMELSSLRSEDTAVYYCARGKYWMYDWGK | |
| | GTLVTVSS | |
| 108 | QSVLTQPPSVSAAPGQKVTISCSGGSSNIGNSYVSWYQQLPGTAPKLLIYDNNKRPS | MEDI-21-VL |
| | GIPDRFSGSKSGTSATLAITGLQTGDEAVYFCGTWDTSTVWEWPFGTGTKLTVL | |
| 109 | QVQLVQSGAEVKKPGASVKVSCKASGYAFTSYYMHWARQAPGQGLEWMGIINPS | MEDI-22-VH |
| | GGSTSYAQKFQGRVTMTRDTSTSTVYMELSSLRSEDTAVYYCARGKYWMYDWGKG | |
| | TLVTVSS | |
| 110 | QPVLTQPPSVSAAPGQKVTISCSGGSSNIGNSYVSWYQQLPGTAPKLLIYDNNKRPS | MEDI-22-VL |
| | GIPDRFSGSKSGTSATLAITGLQTGDEADYFCGTWDTSTVWEWPFGTGTKLTVL | |
| 111 | QVQLVQSGAEVRKPGASVKVSCKASGYAFTSYYMHWARQAPGQGLEWMGIINPS | MEDI-23-VH |
| | GGSTSYAQKFQGRVTMTRDTSTSTVYMELSSLRSEDTAVYYCARGKYWMYDWGKG | |
| | TLVTVSS | |
| 112 | QSVLTQPPSVSAAPGQKVTISCSGGSSNIGNNYVSWYQQLPGTAPKLLIYDNNKRPP | MEDI-23-VL |
| | GIPDRFSGSKSGTSATLAITGLQTGDEADYYCGTWDTSTVWEWPFGTGTKLTVL | |
| 113 | QVQLVQSGAEVKKPGASVKVSCKASGYAFTSYYMHWARQAPGQGLEWMGIINPR | MEDI-24-VH |
| | GGSTSYAQKFQGRVTMTRDTSTSTVYMELSSLRSEDTAVYYCARGKYWMYDWGKG | |
| | TLVTVSS | |
| 114 | QSVLTQPPSVSAAPGQKVTISCSGGSSNIGNSYVSWYQQLPGTAPKLLIYDNNKRPS | MEDI-24-VL |
| | GIPDRFSGSKSGTSATLAITGLQTGDEADYFCGTWDTSTVWEWPFGTGTKLTVL | |
| 115 | QVQLVQSGAEVKKPGASVKVSCKASGYAFTSYYMHWARQAPGQGLEWMGIINPR | MEDI-25-VH |
| | GGSASYAQKFQGRVSMTRDTSTSTVYMELSSLRSEDTAVYYCARGKYWMYDWGK | |
| | GTLVTVSS | |
| 116 | QSVLTQPPSVSAAPGQKVTISCSGGSSNIGNSYVSWYQQLPGTAPKLLIYDNNKRPS | MEDI-25-VL |
| | GIPDRFSGSKSGTTATLAITGLQTGDEADYYCGTWVTSTVWEWPFGTGTKLTVL | |
| 117 | QVQLVQSGAEVKKPGASVKVSCKASGYAFTSYYMHWARQAPGQGLEWMGIINPS | MEDI-26-VH |
| | GGSTSYAQKFQGRVTMTRDTSTSTVYMELSSLRSEDTAVYYCARGKYWMYDWGKG | |
| | TLVTVSS | |
| 118 | QSVLTQPPSVSAAPGQKVTISCSGGSSNIGNSYVSWYQQLPGTAPKLLIYDNNKRPS | MEDI-26-VL |
| | GIPDRFSGSKSGTSATLAITGLQTGDEADYFCGTWDTSTVWEWPFGTGTKLTVL | |
| 119 | QVQLVQSGAEVRKPGASVKVSCKASGYAFTSYYMHWARQAPGQGLEWMGIINPS | MEDI-27-VH |
| | GGSTSYAQKFQGRVTMTRDTSTSTVYMELSSLRPEDTAVYYCARGKYWMYDWGK | |
| | GTQVTVSS | |
| 120 | QSVLTQPPLVSAAPGQKVTISCSGGSSNIGNSYVSWYQRLPGTAPKLLIYDNNKRPSG | MEDI-27-VL |
| | IPDRFSGSKSGTSATLAITGLQTGDEADYYCGTWDTSTVWEWPFGTGTKLTVL | |
| 121 | QVQLVQSGAEVKKPGASVKVSCKASGYAFTSYYMHWARQAPGQGLEWMGIINPS | MEDI-28-VH |
| | GGSTSYAQKFQGRVTMTRDTSTSTVYMELSSLRSEDTAVYYCARGKYWMYDWGN | |
| | GTLVTVSS | |
| 122 | LPVLTQPPSVSAAPGQKVTISCSGGSSSIGNSYVSWYQQLPGAAPKLLIYDNNKRPSG | MEDI-28-VL |
| | IPDRFSGFRSGTSATLAITGLQTGDEADYYCGTWDTSPVWEWPFGTGTKLTVL | |
| 123 | QVQLVQSGAEVKKPGASVKVSCKASGYAFTSYYMHWARQAPGQGLEWMGIINPS | MEDI-29-VH |
| | GGSTSYAQKFQGRVTMTRDTSTSTVYMELSSLRSEDTAVYYCARGKYWMYDWGKG | |
| | TRVTVSS | |
| 124 | QSVLTQPPSVSAAPGQKVTISCSGGSSNIGNSYVSWYQQLPGTAPKLLIYDNNKRPS | MEDI-29-VL |
| | GIPDRFSGSKSGTSATLAITGLQTGDEADYYCGTWDTSPVWEWPFGTGTKLTVL | |
| 125 | QVQLVQSGAEVKKPGASVKVSCKASGYAFTSYYMHWARQAPGQGLEWMGIINPS | MEDI-30-VH |
| | GGSTSYAQKFQGRVTMTRDTSTSTVYMELSSLRSEDTAVYYCARGKYWMYDWGKG | |
| | TLVTVSS | |
| 126 | QSVLTQPPSVSAAPGQKVTISCSGGSSNIGNSYVSWYQRLPGAAPKLLIYDNNKRPS | MEDI-30-VL |
| | GIPDRFSGSKSGTSATLAITGLQTGDEADYYCGTWDTSTVWEWPFGTGTKLTVL | |
| 127 | QVQLVQSGAEVKKPGASVKVSCKASGYAFTSYYMHWARQAPGQGLEWMGIINPS | MEDI-31-VH |
| | GGSTSYAQKFQGRVTMTRDTSTSTVYMELSSLRSEDTAVYYCARGKYWMYDWGKG | |
| | TLVTVSS | |
| 128 | QSVLTQPPSVSAAPGQKVTISCSGGSSSIGNSYVSWYQQLPGTAPKLLIYDNNKRPSG | MEDI-31-VL |
| | IPDRFSGSKSGTSATLAITGLQTGDEADYYCGTWATSPVWEWPFGTGTKLTVL | |
| 129 | QVQLVQSGAEVKKPGASVKVSCKASGYAFTSYYMHWARQAPGQGLEWMGIINPS | MEDI-32-VH |
| | GGSTSYAQKFQGRVTMTRDTSTSTVYMELSSLRSEDTAVYYCARGKYWMYDWGKG | |
| | TLVTVSS | |
| 130 | QSVLTQPPSVSAAPGQKVTISCSGGSSNIGNSYVSWYQQLPGTAPKLLIYDNNKRPS | MEDI-32-VL |
| | GIPDRFSGSKSGTSATLAITGLQTGDEADYFCGTWDTSTAWEWPFGTGTKLTVL | |
| 131 | QVQLVQSGAEEKKPGASVKVSCKASGYAFTSYYMHWARQAPGQGLEWMGIINPS | MEDI-33-VH |
| | GGSTSYAQKFQGRVTMTRDTSTSTVYMELSSLRSEDTAVYYCARGKYWMYDWGKG | |
| | TLVTVSS | |
| 132 | QSALTQPPSVSAAPGQKVTISCSGGSSNIGNSYVSWYQQLPGTAPKLLIYDNNKRPS | MEDI-33-VL |
| | GIPDRFSGSKSGTSATLAITGLQTGDEADYFCGTWDTSTVWEWPFGTGTKLTVL | |
| 133 | QVQLVQSGAEVKKPGASVKVSCKASGYAFTSYYMHWARQAPGQGLEWMGIINPS | MEDI-34-VH |
| | GGSTSYAQKFQGRVSMTRDTSTSTVYMELSSLRSEDTAVYYCARGKYWMYDWGKG | |
| | TLVTVSS | |
| 134 | QSVLTQPPSVSAAPGQKVTISCSGGSSNIGNSYVSWYQQLPGTAPKLLIYDNNKRPS | MEDI-34-VL |
| | GIPDRFSGSKSGTSATLAITGLQTGDEADYFCGTWDTSTVWEWPFGTGTKLTVL | |
| 135 | QVQLVQSGAEVKKPGASVKVSCKASGYAFTSYYMHWARQAPGQGLEWMGIINPS | MEDI-35-VH |
| | GGSTSYAQKFQGRVTMTRDTSTSTVYMELSSLRSEDTAVYYCARGKYWMYDWGKG | |
| | TLVTVSS | |
| 136 | QSVLTQPPSVSAAPGQKVTISCSGGSSNIGNSYVSWYQQLPGTAPKLLIYDNNKRPS | MEDI-35-VL |
| | GIPDRFSGSKSGTSATLAITGLQTGDEADYYCGTWDTSPVWEWPFGTGTKLTVL | |
| 137 | QVQLVQSGAEVKKPGASVKVSCKASGYAFTSYYMHWARQAPGQGLEWMGIINPS | MEDI-36-VH |
| | GGSASYAQKFQGRVTMTRDTSTSTVYMELSSLRSEDTAVYYCARGKYWMYDWGK | |
| | GTLVTVSS | |
| 138 | QSVLTQPPSVSAAPGQKVTISCSGGSSNIGNSYVSWYQQLPGTAPKLLIYDNNKRPS | MEDI-36-VL |
| | GIPDRFSGSKSGTSATLAITGLQTGDEADYYCGTWDSSTVWEWPFGTGTKLTVL | |
| 139 | QVQLVQSGAEVKKPGASVKVSCKASGYAFTSYYMHWARQAPGQGLEWMGIINPR | MEDI-37-VH |
| | GGSTSYAQKFQGRVAMTRDTSTSTVYMELSSLRPEDTAVYYCARGKYWMYDWGK | |
| | GTLVTVSS | |
| 140 | QSVLTQPPSVSAAPGQKVTISCSGGGSSIGNSYVSWYQQLPGTAPKLLIYDNNKRPS | MEDI-37-VL |
| | GVPDRFSGSKSGTSATLAITGLQTGDEADYYCGTWDTSPVWEWPFGTGTKLTVL | |
| 141 | QVQLVQSGAEVKKPGASVKVSCKASGYAFTSYYMHWARQAPGQGLEWMGIINPS | MEDI-38-VH |
| | GGSASYAQKFQGRVTMTRDTSTSTVYMELSSLRSEDTAVYYCARGKYWMYDWGK | |
| | GTLVTVSS | |
| 142 | QSVLTQPPSVSAAPGQKVTISCSGGSSNIGNSYVSWYQQLPGTAPKLLIYDNNKRPS | MEDI-38-VL |
| | GIPDRFSGSKSGTSATLAITGLQTGDEADYFCGTWDTSTVWEWPFGTGTKLTVL | |
| 143 | QVQLVQSGAEVKKPGASVKVSCKASGYAFTSYYMHWARQAPGQGLEWMGIINPR | MEDI-39-VH |
| | GGSTSYAQKFQGRVTMTRDTSTSTVYMELSSLRSEDTAVYYCARGKYWMYDWGKG | |
| | TLVTVSS | |
| 144 | QSVLTQPPSVSAAPGQKVTISCSGGSSNIGNSYVSWYQQLPGTAPKLLIYDNNKRPS | MEDI-39-VL |
| | GIPDRFSGSKSGTSATLAITGLQTGDEADYYCGTWDTSTAWEWPFGTGTKLTVL | |
| 145 | QVQLVQSGAEVKKPGASVKVSCKASGYAFTSYYMHWARQAPGQGLEWMGIINPS | MEDI-40-VH |
| | GGSTSYAQKFQGRVTMTRDTSTSTVYMELSSLRSEDTAVYYCARGKYWMYDWGKG | |
| | TLVTVSS | |
| 146 | QSVLTQPPSVSAAPGQKVTISCSGGSSNIGNSYVSWYQQLPGTAPKLLIYDNNKRPS | MEDI-40-VL |
| | GIPDRFSGSKSGTSATLAITGLQTGDEADYYCGTWDSSTVWEWPFGTGTKLTVL | |
| 147 | QVQLVQSGAEVRKPGASVKVSCKASGYAFTSYYMHWARQAPGQGLEWMGIINPS | MEDI-41-VH |
| | GGSTSYAQKFQGRVTMTRDTSTSTVYMELSSLRPEDTAVYYCARGKYWMYDWGK | |
| | GTLVTVSG | |
| 148 | QSVLTQPPSVSAAPGQKVTISCSGGSTNIGNSYVSWYQRLPGTAPKLLIYDNNKRPP | MEDI-41-VL |
| | GIPDRFSGSKSGTSATLAITGLQTGDEADYYCGTWDTSTVWEWPFGTGTKLTVL | |
| 149 | QVQLVQSGAEVKKPGASVKVSCKASGYAFTSYYMHWARQAPGQGLEWVGIINPSG | MEDI-42-VH |
| | GSTSYAQKFQGRVTMTRDTSTSTVYMELSSLRSGDTAVYYCARGKYWMYDWGKGT | |
| | LVTVSS | |
| 150 | QAVLTQPPSVSAAPGQKVTISCSGGSSNIGNSYVSWYQRLPGAAPKLLIYDNNKRPS | MEDI-42-VL |
| | GIPDRFSGSKSGTSATLAITGLQTGDEADYYCGTWDTSTGWEWPFGTGTKLTVL | |
| 151 | QVQLVQSGAEVKKPGASVKVSCKASGYAFTSYYMHWVRQAPGQGLEWMGIINPR | MEDI-37GL-VH |
| | GGSTSYAQKFQGRVTMTRDTSTSTVYMELSSLRSEDTAVYYCARGKYWMYDWGKG | |
| | TLVTVSS | |
| 152 | QSVLTQPPSVSAAPGQKVTISCSGGGSSIGNSYVSWYQQLPGTAPKLLIYDNNKRPS | MEDI-37GL-VL |
| | GIPDRFSGSKSGTSATLGITGLQTGDEADYYCGTWDTSPVWEWPFGTGTKLTVL | |
| 153 | EVQLLESGGGLVQPGGSLRLSCAVSGFTFSNYAMSWVRQAPGKGLEWVSAISSGGG | AJOU-1-VH |
| | NIYYADSVKGRFTISRDNSKNTLYLQMNSLRAEDTAVYYCAKLRRYFDYWGQGTLVT | |
| | VSS | |
| 154 | EVQLLESGGGLVQPGGSLRLSCAASGFTFSDYAMSWVRQAPGKGLEWVSAISSGGS | AJOU-2-VH |
| | SIYYADSVKGRFTISRDNSKNTLHLQMNSLRAEDTAVYYCARGPQRSATAVFDYWG | |
| | QGTLVTVSS | |
| 155 | EVQLLESGGGLVQPGGSLRLSCAASGFTFSNYAMSWVRQAPGKGLEWVSWISPNS | AJOU-3-VH |
| | GNIYYADSVKGRFTISRDNSKNTLYLQMNSLRAEDTAVYYCARRPLSAAWSHSSYYN | |
| | AMDVWGQGTLVTVSS | |
| 156 | EVQLLESGGGLVQPGGSLRLSCAASGFTFSGYAMSWVRQAPGKGLEWVSLISHSGS | AJOU-4-VH |
| | NTYYADSVKGRFTISRDNSKNTLYLQMNSLRAEDTAVYYCARPHRAFDYWGQGTLV | |
| | TVSS | |
| 157 | EVQLLESGGGLVQPGGSLRLSCAASGFTFSNYAMSWVRQAPGKGLEWVSGISHGS | AJOU-5-VH |
| | GSIYYADSVKGRFTISRDNSKNTLYLQMNSLRAEDTAVYYCARPHRAFDYWGQGTLV | |
| | TVSS | |
| 158 | EVQLLESGGGLVQPGGSLRLSCAASGFTFSNYAMSWVRQAPGKGLEWVSGISHGN | AJOU-6-VH |
| | GSIYYADSVKGRFTISRDNSKNTLYLQMNSLRAEDTAVYYCAKTGRHFDYWGQGTLV | |
| | TVSS | |
| 159 | EVQLLESGGGLVQPGGSLRLSCAASGFTFSNYAMSWVRQAPGKGLEWVSSISPSGS | AJOU-7-VH |
| | SIYYADSVKGRFTISRDNSKNTLYLQMNSLRAEDTAVYYCARSYRAFDYWGQGTLVT | |
| | VSS | |
| 160 | EVQLLESGGGLVQPGGSLRLSCAASGFTFSNYAMSWVRQAPGKGLEWVSAISPSGG | AJOU-8-VH |
| | SIYYADSVKGRFTISRDNSKNTLYLQMNSLRAEDTAVYYCARAKRAFDYWGQGTLVT | |
| | VSS | |
| 161 | EVQLLESGGGLVQPGGSLRLSCAASGFTFSNYAMSWVRQAPGKGLEWVSAISPGSG | AJOU-9-VH |
| | STYYADSVKGRFTISRDNSKNTLYLQMNSLRAEDTAVYYCAKFRRHFDYWGQGTLVT | |
| | VSS | |
| 162 | EVQLLESGGGLVQPGGSLRLSCAASGFTFSNYAMSWVRQAPGKGLEWVSAISSGGG | AJOU-10-VH |
| | NIYYADSVKGRFTISRDNSKNTLYLQMNSLRAEDTAVYYCARVHRAFDYWGQGTLV | |
| | TVSS | |
| 163 | EVQLLESGGGLVQPGGSLRLSCAASGFTFSNYAMSWVRQAPGKGLEWVSAITSSGR | AJOU-69-VH |
| | SIYYADSVKGRFTISRDNSKNTLYLQMNSLRAEDTAVYYCARVHRAFDYWGQGTLVT | |
| | VSS | |
| 164 | EVQLLESGGGLVQPGGSLRLSCAASGFTFSNYAMSWVRQAPGKGLEWVSAITSSGA | AJOU-70-VH |
| | NIYYADSVKGRFTISRDNSKNTLYLQMNSLRAEDTAVYYCARVHRAFDYWGQGTLV | |
| | TVSS | |
| 165 | EVQLLESGGGLVQPGGSLRLSCAASGFTFSNYAMSWVRQAPGKGLEWVSAITSSGG | AJOU-71-VH |
| | NIYYADSVKGRFTISRDNSKNTLYLQMNSLRAEDTAVYYCARVHRAFDYWGQGTLV | |
| | TVSS | |
| 166 | EVQLLESGGGLVQPGGSLRLSCAASGFTFSNYAMSWVRQAPGKGLEWVSAITAGG | AJOU-72-VH |
| | GSIYYADSVKGRFTISRDNSKNTLYLQMNSLRAEDTAVYYCARVHRAFDYWGQGTLV | |
| | TVSS | |
| 167 | EVQLLESGGGLVQPGGSLRLSCAASGFTFSRHAMAWVRQAPGKGLEWVSAITSSGR | AJOU-83-VH |
| | SIYYADSVKGRFTISRDNSKNTLYLQMNSLRAEDTAVYYCARVHRAFDYWGQGTLVT | |
| | VSS | |
| 168 | QSVLTQPPSASGTPGQRVTISCSGSSSNIGNNYVNWYQQLPGTAPKLLIYDNSHRPS | AJOU-33-VL |
| | GVPDRFSGSKSGTSASLAISGLRSEDEADYYCGTWDASLSAYVFGGGTKLTVL | |
| 169 | QSVLTQPPSASGTPGQRVTISCSGSSSNIGNNNVSWYQQLPGTAPKLLIYANSKRPS | AJOU-34-VL |
| | GVPDRFSGSKSGTSASLAISGLRSEDEADYYCGSWDDSLSAYVFGGGTKLTVL | |
| 170 | QSVLTQPPSAPGTPGQRVTISCTGSSSNIGSNSVNWYQQLPGTAPKLLIYDDSHRPS | AJOU-35-VL |
| | GVPDRFSGSKSGTSASLAISGLRSEDEADYYCDAWDSSLSAYVFGGGTKLTVL | |
| 171 | QSVLTQPPSASGTPGQRVTLSCTGSSSNIGSNYVSWYQQLPGTAPKLLIYADSQRPSG | AJOU-36-VL |
| | VPDRFSGSKSGTSASLAISGLRSEDEADYYCGTWDDSLSGYVFGGGTKLTVL | |
| 172 | QSVLTQPPSASGTPGQRVTISCSSSSSNIGSNYVSWYQQLPGTAPKLLIYSDSHRPSG | AJOU-37-VL |
| | VPDRFSGSKSGTSASLAISGLRSEDEADYYCGSWDYSLSAYVFGGGTKLTVL | |
| 173 | QSVLTQPPSASGTPGQRVTISCTGSSSNIGNNTVSWYQQLPGTAPKLLIYDNSHRPS | AJOU-38-VL |
| | GVPDRFSGSKSGTSASLAISGLQSEDEADYYCGSWDYSLSAYVFGGGTKLTVL | |
| 174 | QSVLTQPPSASGTPGQRVTISCTGSSSNIGNNDVNWYQQLPGTAPKLLIYYDSQRPS | AJOU-39-VL |
| | GVPDRFSGSKSGTSASLAISGLRSEDEADYYCATWDASLSAYVFGGGTKLTVL | |
| 175 | QSVLTQPPSASGTPGQRVTISCSGSSSNIGSNAVNWYQQLPGTAPKLLIYYDNQRPS | AJOU-40-VL |
| | GVPDRFSGSKSGTSASLAISGLRSEDEADYYCGTWDDSLNGYVFGGGTKLTVL | |
| 176 | QSVLTQPPSASGTPGQRVTISCSGSSSNIGNNAVTWYQQLPGTAPKLLIYDDSHRPS | AJOU-41-VL |
| | GVPDRFSGSKSGTSASLAISGLRSEDEADYYCGSWDYSLSAYVFGGGTKLTVL | |
| 177 | QSVLTQPPSASGTPGQRVTISCSGSSSNIGSNTFNWYQQLPGTAPKLLIYADSHRPSG | AJOU-42-VL |
| | VPDRFSGSKSGTSASLAISGLRSEDEADYYCGTWDYSLSGYVLGGGTKLTVL | |
| 178 | QSVLTQPPSASGTPGQRVTISCSGSSSNIGSNTFNWYQQLPGTAPKLLIYADSHRPSG | AJOU-77-VL |
| | VPDRFSGSKSGTSASLAISGLRSEDEADYYCGTWDYSLSGYVLGGGTKLTVL | |
| 179 | QSVLTQPPSASGTPGQRVTISCSGSSSNIGSNTFNWYQQLPGTAPKLLIYADSHRPSG | AJOU-78-VL |
| | VPDRFSGSKSGTSASLAISGLRSEDEADYYCGTWDYSLRGYVLGGGTKLTVL | |
| 180 | QSVLTQPPSASGTPGQRVTISCSGSSSNIGSNTFNWYQQLPGTAPKLLIYADSHRPSG | AJOU-79-VL |
| | VPDRFSGSKSGTSASLAISGLRSEDEADYYCGYWDYSLSGYVLGGGTKLTVL | |
| 181 | QSVLTQPPSASGTPGQRVTISCSGSSSNIGSNTFNWYQQLPGTAPKLLIYADSHRPSG | AJOU-80-VL |
| | VPDRFSGSKSGTSASLAISGLRSEDEADYYCGTWDYSLSGYVLGGGTKLTVL | |
| 182 | QSVLTQPPSASGTPGQRVTISCSGSSANSRTDGFNWYQQLPGTAPKLLIYADSHRPS | AJOU-86-VL |
| | GVPDRFSGSKSGTSASLAISGLRSEDEADYYCGTWDYSLSGYVLGGGTKLTVLG | |
| 183 | QSVLTQPPSASGTPGQRVTISCSGSAQFGSRDNFNWYQQLPGTAPKLLIYADSHRPS | AJOU-87-VL |
| | GVPDRFSGSKSGTSASLAISGLRSEDEADYYCGTWDYSLSGYVLGGGTKLTVLG | |
| 184 | QSVLTQPPSASGTPGQRVTISCSGSTKQMHNYQFNWYQQLPGTAPKLLIYADSHRP | AJOU-88-VL |
| | SGVPDRFSGSKSGTSASLAISGLRSEDEADYYCGTWDYSLSGYVLGGGTKLTVLG | |
| 185 | QSVLTQPPSASGTPGQRVTISCSGSLLRGENLQFNWYQQLPGTAPKLLIYADSHRPS | AJOU-89-VL |
| | GVPDRFSGSKSGTSASLAISGLRSEDEADYYCGTWDYSLSGYVLGGGTKLTVLG | |
| 186 | QSVLTQPPSASGTPGQRVTISCSGSPLFPDSGSFNWYQQLPGTAPKLLIYADSHRPSG | AJOU-90-VL |
| | VPDRFSGSKSGTSASLAISGLRSEDEADYYCGTWDYSLSGYVLGGGTKLTVLG | |
| 187 | QSVLTQPPSASGTPGQRVTISCSGSAALDLSPSFNWYQQLPGTAPKLLIYADSHRPSG | AJOU-91-VL |
| | VPDRFSGSKSGTSASLAISGLRSEDEADYYCGTWDYSLSGYVLGGGTKLTVLG | |
| 188 | QVQLVQSGAEVKKPGASVKVSCKASGYTFTNYGISWVRQAPGQGLEWMGWISVY | REGN-VH-3 |
| | NGKTNYAQKLQGRVTMTTDTSTTTAYMEMRSLRSDDTAVYYCARGSGYDLDYWG | |
| | QGTLVSVSS | |
| 189 | EVQLVESGGGLVQPGGSLRLSCAASGFTFSSFWMTWVRQAPGKGLEWVANIKQD | REGN-VH-19 |
| | GSEKYYVDSVKGRFTISRDNAKNSLYLQMNSLRAEDTAVYYCARDPGRTMVRGGIRY | |
| | YYGMDVWGQGTTVTVSS | |
| 190 | EVKLAESGGGLVQPGGSLRLSCAASGFTFSSHWMNWVRQAPGKGLEWVANIKQD | REGN-VH-35 |
| | GSDKYYVDSVKGRFTISRDNAKNSLYLQLNSLIAEDTAVYYCARDRGVRPPRGAFDIW | |
| | GQGTMVTVSS | |
| 191 | QVQLVQSGAEVKKPGASVKVSCKASGYTFNSYGISWVRQAPGQGLEWMGWIRTY | REGN-VH-51 |
| | NGNTNYAQKLQGRVTMTTDTSTSTAYMELRSLRSDDTAVYYCARDEARIVVAGTTP | |
| | YYYGMDVWGQGTTVTVSS | |
| 192 | QVQLVESGGGLVQPGGSLRLSCAVSGFTISDHYMSWIRQAPGKGLEWISYISSSGSKI | REGN-VH-67 |
| | YYADSVKGRFTISRDNAKNSLFLQMNSLRAEDTAVYYCARTRQLVGDYWGQGTLVT | |
| | VSS | |
| 193 | EVQLVESGGGLVQPGRSLRLSCAASGFTFDNYAMHWVRQAPGKGLEWVSGIRWN | REGN-VH-83 |
| | SGSIGYADSVKGRFTISRDNAKNSLYLQMNSLRAEDTALYYCAKEGGYSGYRPGPFFD | |
| | YWGQGTLVTVSS | |
| 194 | QVQLVQSGAEVKKPGASVKVSCKASGYTFTNYGISWVRQAPGQGLEWMGWISVY | REGN-VH-99 |
| | NGHTNYAQKLQGRVTMTTDTSTSTAYMELRSLRSDDTAVYYCARGSGYDFDSWGQ | |
| | GTLVTVSS | |
| 195 | QVQLVQSGAEVKKPGASVKVSCKASRYTFTSYDINWVRQATGQGLEWMGWMNP | REGN-VH-115 |
| | NSGNTGYAQKFQGRVTMTRNTSTSTAYMELSSLRSEDTAVYYCARVRRFFDYWGQ | |
| | GTLVTVSS | |
| 196 | QVQLVQSGPEVKKPGASVKVSCKASGYTFTNYGISWVRQAPGQGLEWMGWISVY | REGN-VH-147 |
| | NGNINYAQKLQGRVTMTTDTSTSTAYMDLRSLRSDDTAVYYCARGSGYDFDYWGQ | |
| | GTLVTVSS | |
| 197 | QVQLVQSGAEVKKPGASVKVSCKDSAYTFNRYGISWVRQAPGQGLEWMGWISAY | REGN-VH-163 |
| | TGNTVYAQKLQGRVTMTTDNSTSTAYMELRSLRSDDTAVYYCARDKSIFGVVRGFD | |
| | YWGQGTLVTVSS | |
| 198 | AIQMTQSPSSLSASVGDRVTITCRASQGIRNALGWYQQKPGKAPKLLIYAASSLQSG | REGN-VL-11 |
| | VPSRFSGSGSGTDFTLTFSSLQPEDFATYYCLQDFNYPYTFGQGTKLEIK | |
| 199 | DIQMTQSPSSVSASVGDRVTISCRASQGVSSWLAWYQQKPGNAPKLLISAASSIQSG | REGN-VL-27 |
| | VPSRFSGSGSGTDFTLTISSLQPEDFATYYCQQANSFPLTFGGGTKVEIK | |
| 200 | DIQMTQSPSSVSASVGDRVTITCRASQGISSWLAWYQQKPGKAPKLLIYAASSFQSG | REGN-VL-43 |
| | VPSRFSGSGSGTDFTLTISSLQPEDFATYFCQQANSFPLTFGGGTTVEIK | |
| 201 | DIQMTQSPSSVSASVGDRVTITCRASQDISIWLAWYQQSPGKAPKLLINVASRLQSG | REGN-VL-59 |
| | VPSRFSGSGSGTDFTLTINSLQPEDFVTYYCQQANSFPITFGQGTRLATK | |
| 202 | DIQLTQSPSFLSASVGDRVTITCWASQGISSYLAWYQQKPGKAPKLLIFAASTLQSGV | REGN-VL-75 |
| | PSRFSGSGSGTEFTLTISSLQPEDFATYYCQQLNSYPLTFGGGTKVEIR | |
| 203 | EIVMTQSPATLSVSPGERATLSCRASQSVNYNLAWYQHKPGQAPRLLIYGASTRATGI | REGN-VL-91 |
| | PARFSGSGSGTEFTLTISSLQSEDFAVYYCQQYNNWPLTFGGGTKVEIK | |
| 204 | AIQMTQSSSSLSASVGDRVTITCRASQAIRNALGWYQQKPGKAPKVLIYAASSLQSGI | REGN-VL-107 |
| | PSRFSGSGSGTDFTLTISSLQPEDFATYYCLQDYDYPYTFGQGTKLEIK | |
| 205 | DIQLTQSPSFLSASVGDRVTITCWASQGIISYLAWYQQKPGKAPKLLIYAASTLHSGVP | REGN-VL-123 |
| | SRFSGSGSGTEFTLTISSLQPEDFATYYCHQLKSYPITFGQGTRLEIK | |
| 206 | AIQMTQSPSSLSASVGDRVTITCRASQDIRNALGWYQQKPGKAPKLLIYAASSLQSG | REGN-VL-155 |
| | VPSRFSGSASGTDFTLTISSLQPEDFAAYYCLQDYNYPYTFGQGTKLEIK | |
| 207 | EIVMTQSPVTLSLSPGERATLPCRASQSVSSSLAWYQQKAGQSPRLLIYGASTRATGI | REGN-VL-171 |
| | PARFSGSGSGTEFTLTISNLQSEDFAVYYCQQYNNWPLTFGGGTKVEIK | |
| 208 | EVQLLESGGGLVQPGGSLRLSCAASGFTLSSYAMHWVRQAPGKGLEYVSGISSNGG | STSA-C27-VH |
| | STYYANSVKGRFTISRDNPKNTLFLQMSSLRAEDTAVYYCVRVKVGYRGGMDVWG | |
| | QGTTVTVSS | |
| 209 | EVQLLESGGGLVQPGGSLRLSCAASGFTLSSYAMHWVRQAPGKGLEYVSGISPSGSS | STSA-C27-6-33-VH |
| | TYYANSVKGRFTISRDNPKNTLFLQMSSLRAEDTAVYYCVRSKVRYRGGMDVWGQ | |
| | GTTVTVSS | |
| 210 | EVQLLESGGGLVQPGGSLRLSCAASGFTLSSYAMHWVRQAPGKGLEYVSGISPSGVS | STSA-C27-7-33-VH |
| | TYYANSVKGRFTISRDNPKNTLFLQMSSLRAEDTAVYYCVRVKVKYRGGMDVWGQ | |
| | GTTVTVSS | |
| 211 | EVQLLESGGGLVQPGGSLRLSCAASGFTLSSYAMHWVRQAPGKGLEYVSGISPTSGS | STSA-C27-24-56-VH |
| | TYYANSVKGRFTISRDNPKNTLFLQMSSLRAEDTAVYYCVRVKVRYRGGMDVWGQ | |
| | GTTVTVSS | |
| 212 | EVQLLESGGGLVQPGGSLRLSCAASGFTLSSYAMHWVRQAPGKGLEYVSGISPTGTS | STSA-C27-47-56-VH |
| | TYYANSVKGRFTISRDNPKNTLFLQMSSLRAEDTAVYYCVRVKGAYRGGMDVWGQ | |
| | GTTVTVSS | |
| 213 | EVQLLESGGGLVQPGGSLRLSCAASGFTLSSYAMHWVRQAPGKGLEYVSGISSSGSS | STSA-C27-33-33-VH |
| | TYYANSVKGRFTISRDNPKNTLFLQMSSLRAEDTAVYYCVRVKVAYRGGMDVWGQ | |
| | GTTVTVSS | |
| 214 | EVQLLESGGGLVQPGGSLRLSCAASGFTLSSYAMHWVRQAPGKGLEYVSGISPSSTS | STSA-C27-56-56-VH |
| | TYYANSVKGRFTISRDNPKNTLFLQMSSLRAEDTAVYYCVRVKVLYRGGMDVWGQ | |
| | GTTVTVSS | |
| 215 | EVQLLESGGGLVQPGGSLRLSCAASGFTLSSYAMHWVRQAPGKGLEYVSGISPSSAS | STSA-C27-78-78-VH |
| | TYYANSVKGRFTISRDNPKNTLFLQMSSLRAEDTAVYYCVRVKSKYRGGMDVWGQ | |
| | GTTVTVSS | |
| 216 | EVQLLESGGGLVQPGGSLRLSCAASGFTLSSYAMHWVRQAPGKGLEYVSGISGNSAS | STSA-C27-82-58-VH |
| | TYYANSVKGRFTISRDNPKNTLFLQMSSLRAEDTAVYYCVRVKLKYRGGMDVWGQG | |
| | TTVTVSS | |
| 217 | EVQLLESGGGLVQPGGSLRLSCAASGFTLSSYAMHWVRQAPGKGLEYVSGISHSGTS | STSA-C27-54-54-VH |
| | TYYANSVKGRFTISRDNPKNTLFLQMSSLRAEDTAVYYCVRVRVLYRGGMDVWGQ | |
| | GTTVTVSS | |
| 218 | EVQLLESGGGLVQPGGSLRLSCAASGFTLSSYAMHWVRQAPGKGLEYVSGISPSGVS | STSA-C27-36-36-VH |
| | TYYANSVKGRFTISRDNPKNTLFLQMSSLRAEDTAVYYCVRVKVKYRGGMDVWGQ | |
| | GTTVTVSS | |
| 219 | EVQLLESGGGLVQPGGSLRLSCAASGFTLSSYAMHWVRQAPGKGLEYVSGISSNGG | STSA-C27-53-53-VH |
| | STYYANSVKGRFTISRDNPKNTLFLQMSSLRAEDTAVYYCVRVFVRYRGGMDVWGQ | |
| | GTTVTVSS | |
| 220 | EVQLLESGGGLVQPGGSLRLSCAASGFTLSSYAMHWVRQAPGKGLEYVSGISPTSAS | STSA-C27-67-67-VH |
| | TYYANSVKGRFTISRDNPKNTLFLQMSSLRAEDTAVYYCVRVKGRYRGGMDVWGQ | |
| | GTTVTVSS | |
| 221 | EVQLLESGGGLVQPGGSLRLSCAASGFTLSSYAMHWVRQAPGKGLEYVSGISPTGGS | STSA-C27-55-55-VH |
| | TYYANSVKGRFTISRDNPKNTLFLQMSSLRAEDTAVYYCVRVKGRYRGGMDVWGQ | |
| | GTTVTVSS | |
| 222 | EVQLLESGGGLVQPGGSLRLSCAASGFTLSSYAMHWVRQAPGKGLEYVSGISHSGN | STSA-C27-59-59-VH |
| | STYYANSVKGRFTISRDNPKNTLFLQMSSLRAEDTAVYYCVRVKRRYRGGMDVWGQ | |
| | GTTVTVSS | |
| 223 | EVQLLESGGGLVQPGGSLRLSCAASGFTLSSYAMHWVRQAPGKGLEYVSGISPSSNS | STSA-C27-58-58-VH |
| | TYYANSVKGRFTISRDNPKNTLFLQMSSLRAEDTAVYYCVRVKVRYRGGMDVWGQ | |
| | GTTVTVSS | |
| 224 | EVQLLESGGGLVQPGGSLRLSCAASGFTLSSYAMHWVRQAPGKGLEYVSGISSSGSS | STSA-C27-52-52-VH |
| | TYYANSVKGRFTISRDNPKNTLFLQMSSLRAEDTAVYYCVRVKPAYRGGMDVWGQ | |
| | GTTVTVSS | |
| 225 | EVQLLESGGGLVQPGGSLRLSCAASGFTLSSYAMHWVRQAPGKGLEYVSGISYSSAS | STSA-C27-Y2-Y2-VH |
| | TYYANSVKGRFTISRDNPKNTLFLQMSSLRAEDTAVYYCVRVKVRYRGGMDVWGQ | |
| | GTTVTVSS | |
| 226 | ETTLTQSPDTLPLSPGDRASLSCRASQSVSSAYLAWYQQKPGQAPRLLIYGTSRRATG | STSA-C27-VL |
| | VPGRFSGSGSGTDFTLTISRLEPEDFAVYYCQLYGSSSVTFGQGTKLEIK | |
| 227 | EIVLTQSPGTLSLSPGERATLSCRASQGISSAYLAWYQQKPGQAPRLLIYGTSRRATGI | STSA-C27-6-33-VL |
| | PDRFSGSGSGTDFTLTISRLEPEDFAVYYCQLYGATSVTFGQGTKLEIK | |
| 228 | EIVLTQSPGTLSLSPGERATLSCRASQGISSAYLAWYQQKPGQAPRLLIYGTSRRATGI | STSA-C27-7-33-VL |
| | PDRFSGSGSGTDFTLTISRLEPEDFAVYYCQLYGATSVTFGQGTKLEIK | |
| 229 | EIVLTQSPGTLSLSPGERATLSCRASQSVSSAYLAWYQQKPGQAPRLLIYGTSRRATGI | STSA-C27-24-56-VL |
| | PDRFSGSGSGTDFTLTISRLEPEDFAVYYCQLYGASSVTFGQGTKLEIK | |
| 230 | EIVLTQSPGTLSLSPGERATLSCRASQSVSSAYLAWYQQKPGQAPRLLIYGTSRRATGI | STSA-C27-47-56-VL |
| | PDRFSGSGSGTDFTLTISRLEPEDFAVYYCQLYGASSVTFGQGTKLEIK | |
| 231 | EIVLTQSPGTLSLSPGERATLSCRASQGISSAYLAWYQQKPGQAPRLLIYGTSRRATGI | STSA-C27-33-33-VL |
| | PDRFSGSGSGTDFTLTISRLEPEDFAVYYCQLYGATSVTFGQGTKLEIK | |
| 232 | EIVLTQSPGTLSLSPGERATLSCRASQSVSSAYLAWYQQKPGQAPRLLIYGTSRRATGI | STSA-C27-56-56-VL |
| | PDRFSGSGSGTDFTLTISRLEPEDFAVYYCQLYGASSVTFGQGTKLEIK | |
| 233 | EIVLTQSPGTLSLSPGERATLSCRASQSISTAYLAWYQQKPGQAPRLLIYGTSRRATGIP | STSA-C27-78-78-VL |
| | DRFSGSGSGTDFTLTISRLEPEDFAVYYCQLYGASSVTFGQGTKLEIK | |
| 234 | EIVLTQSPGTLSLSPGERATLSCRASQDISSAYLAWYQQKPGQAPRLLIYGTSRRATGI | STSA-C27-82-58-VL |
| | PDRFSGSGSGTDFTLTISRLEPEDFAVYYCQLYGATSVTFGQGTKLEIK | |
| 235 | EIVLTQSPGTLSLSPGERATLSCRASQDVSSAYLAWYQQKPGQAPRLLIYGTSRRATGI | STSA-C27-54-54-VL |
| | PDRFSGSGSGTDFTLTISRLEPEDFAVYYCQLYGATSVTFGQGTKLEIK | |
| 236 | EIVLTQSPGTLSLSPGERATLSCRASQNISTAYLAWYQQKPGQAPRLLIYGTSRRATGI | STSA-C27-36-36-VL |
| | PDRFSGSGSGTDFTLTISRLEPEDFAVYYCQLYGATSVTFGQGTKLEIK | |
| 237 | EIVLTQSPGTLSLSPGERATLSCRASQDASNAYLAWYQQKPGQAPRLLIYGTSRRATG | STSA-C27-53-53-VL |
| | IPDRFSGSGSGTDFTLTISRLEPEDFAVYYCQLYGSSSVTFGQGTKLEIK | |
| 238 | EIVLTQSPGTLSLSPGERATLSCRASQGVSSAYLAWYQQKPGQAPRLLIYGTSRRATGI | STSA-C27-67-67-VL |
| | PDRFSGSGSGTDFTLTISRLEPEDFAVYYCQLYGRSSVTFGQGTKLEIK | |
| 239 | EIVLTQSPGTLSLSPGERATLSCRASQNISTAYLAWYQQKPGQAPRLLIYGTSRRATGI | STSA-C27-55-55-VL |
| | PDRFSGSGSGTDFTLTISRLEPEDFAVYYCQLYGTSSVTFGQGTKLEIK | |
| 240 | EIVLTQSPGTLSLSPGERATLSCRASQSVSTAYLAWYQQKPGQAPRLLIYGTSRRATGI | STSA-C27-59-59-VL |
| | PDRFSGSGSGTDFTLTISRLEPEDFAVYYCQLYGATSVTFGQGTKLEIK | |
| 241 | EIVLTQSPGTLSLSPGERATLSCRASQDISSAYLAWYQQKPGQAPRLLIYGTSRRATGI | STSA-C27-58-58-VL |
| | PDRFSGSGSGTDFTLTISRLEPEDFAVYYCQLYGATSVTFGQGTKLEIK | |
| 242 | EIVLTQSPGTLSLSPGERATLSCRASQGVSTAYLAWYQQKPGQAPRLLIYGTSRRATGI | STSA-C27-52-52-VL |
| | PDRFSGSGSGTDFTLTISRLEPEDFAVYYCQLYGATSVTFGQGTKLEIK | |
| 243 | EIVLPQSPGTLSLSPGERATLSCRASQGVSSAYLAWYQQKPGQAPRLLIYGTSRRATGI | STSA-C27-Y2-Y2-VL |
| | PDRFSGSGSGTDFTLTISRLEPEDFAVYYCQLYGSTSVTFGQGTKLEIK | |
| 244 | EVQLVESGGGLVQPKGSLKLSCAASGFTFNTYGMHWVRQAPGKGLEWVAHIRSKS | Y0188-1 VH |
| | SNYATYYADSVKDRFTISRDDSQSMLYLQMNNLKTEDTAMYYCVRWFRAMDYWG | |
| | QGTSVTVSS | |
| 245 | EVQLIESGGGLVQPKGSLKLSCAASGFTFNMYAMDWVRQAPGKGLEWVARIRSKG | Y0188-2 VH |
| | SNFETNYADSVKDRFTISRDDSQSMVYLQMINLKTEDTAMYYCVRHRGGAWFAYW | |
| | GQGTLVSVSA | |
| 246 | QVQLVETGGGLVRPGNSLKLSCVTSGFTFSNYRMHWLRQPPGKRLEWIAVITVKSN | Y0188-3 VH |
| | NYGANYAESVKGRFAISRDDSKSSVYLEMNRLREEDTATYFCSRERAYGNPFDYWG | |
| | QGTTLTVSS | |
| 247 | EVQLVESGGGLVQPKGSLKLSCAASGFTFNMYAMNWVRQAPGQGLEWVARIRSKS | Y0188-4 VH |
| | NNYATYYADSVKDRFIISRDDSESMVYLQMSNLRAADTAMYYCVRHLRAMDYWG | |
| | QGTSVTVSS | |
| 248 | EVQLVESGGGLVQPKGSLKLSCAASGFSFNMYAMNWVRQAPGKGLEWVARIRTKS | Y0188-6 VH |
| | NHYSTYYADSVKDRFTISRDDSASMFYLQMNNLKTEDTAMYFCVRHLRAMDYWG | |
| | QGTSVTVSS | |
| 249 | EVQLIESGGGLVQPKGSLKLSCAASGFTFNMYAMDWVRQAPGKGLEWVARIRSKG | Y0188-8 VH |
| | SNFETNYADSVKDRFTISRDDSQSMVYLQMNNLKTEDTAMYYCVRHRGGAWFAY | |
| | WGQGTLVTVSA | |
| 250 | EVQLVESGGGLVRPKGSLKLSCAASGFSFNTYAMNWVRQAPGKGLEWIVWIRSKSH | Y0188-9 VH |
| | NYATYYADSVKDRFTISRDDSESMLYLQMNNLKTEDTAMYYCVRHLRAMDYWGQ | |
| | GTSVTVSS | |
| 251 | EVRLVESGGGLVQPKGSLKLSCEASGFSFNMYAMNWVRQAPGKGLEWITHIRSKSN | Y0188-10 VH |
| | NYATYYADSVKDRFIISRDDSESMVYLQMNNLKTEDTAMYYCVRLLRALDYWGQGT | |
| | SVTVSS | |
| 252 | EVQLVESGGGLVQPKGSLKLSCAASGFTFNMYGMHWVRQAPGKGLEWVAHIRSKS | Y0188-14 VH |
| | SNYATYYADSVKDRLTISRDDSQSMLYLQMNNLKTEDTAMYYCVRWFRAMDYWG | |
| | QGTSVTVSS | |
| 253 | EVQLVESGGGLVKPGGSLRLSCAASGFTFSMYGMHWVRQAPGKGLEWVGHIRSKS | HV3-15-14 VH |
| | SNYATYYADSVKDRFTISRDDSKNTLYLQMNSLKTEDTAVYYCTTWFRAMDYWGQ | |
| | GTLVTVSS | |
| 254 | EVQLVESGGGLVQPGGSLRLSCAASGFTFSMYGMHWVRQAPGKGLEWVSHIRSKS | HV3-48-14 VH |
| | SNYATYYADSVKDRFTISRDNAKNSLYLQMNSLRAEDTAVYYCARWFRAMDYWGQ | |
| | GTLVTVSS | |
| 255 | EVQLVESGGGLVQPGGSLKLSCAASGFTFSMYGMHWVRQASGKGLEWVGHIRSKS | HV3-73*2-14 VH |
| | SNYATYYADSVKDRFTISRDDSKNTAYLQMNSLKTEDTAVYYCTRWFRAMDYWGQ | |
| | GTLVTVSS | |
| 256 | EVQLVESGGGLVQPGGSLRLSCAASGFTFSMYGMHWVRQAPGKGLEWVGHIRSKS | HV3-72-14 VH |
| | SNYATYYADSVKDRFTISRDDSKNSLYLQMNSLKTEDTAVYYCARWFRAMDYWGQ | |
| | GTLVTVSS | |
| 257 | EVQLVESGGGLVQPGGSLRLSCAASGFTFSMYGMHWVRQAPGKGLEWVSHIRSKS | Y01-14 VH |
| | SNYATYYADSVKDRFTISRDNAKNSLYLQMNSLRAEDTAVYYCARWFRAMDYWGQ | |
| | GTLVTVSS | |
| 258 | EVQLVESGGGLEQPGGSLRLSCAGSGFTFRMYGMHWVRQAPGKGLEWVSHIRSKS | 162-14 VH |
| | SNYATYYADSVKDRFTISRDNSKNTLYLQMNSLRAEDTAVYYCAKWFRAMDYWGQ | |
| | GTTVTVSS | |
| 259 | EVQLVESGGGLVQPGGSLKLSCAASGFTFSMYGMHWVRQASGKGLEWVGHIRSKS | VH73-14 VH |
| | SNYATYYADSVKDRFTISRDDSKNTAYLQMNSLKTEDTAVYYCTRWFRAMDYWGQ | |
| | GTTVTVSS | |
| 260 | DIVMTQSHKFMSTSVGDRVSITCKASQDVSTAVAWYQEKPGQSPKLLIYWASTRHT | Y0188-1 VL |
| | GVPDRFTGSGSGTDYTLTISSVQAEDLALYYCQQHYSTPLTFGAGTKLELK | |
| 261 | DIVVTQSPASLAVSLGQRATISCRASKSVSTSGYSYMHWYQQKPGQPPKLLIYLASNL | Y0188-2 VL |
| | ESGVPARFSGSGSGTDFTLNIHPVEEEDVAIYYCQHSRELPLTFGAGTKLELK | |
| 262 | DIQMTQSPSSLSASLGERVSLTCRASQEISGYLSWLQQKPDGTIKRLIYAASTLDSGVP | Y0188-3 VL |
| | KRFSGSRSGSDYSLTISSLESEDFADYYCLQYGSYPYTFGGGTKLEIK | |
| 263 | DIVLTQSPASLTVSLGQRATISCRASKSVSTSGYSYMHWYQQKPGQPPKLLIYLASNLE | Y0188-4 VL |
| | SGVPARFSGSGSGTDFTLNIHPVEEEDAATYYCQHSRELPITFGSGTKLEIK | |
| 264 | DIVLTQSPASLVVSLGQRATISCRASQSVSTSGYSYMHWYQQKPGQPPKLLIYLASNV | Y0188-6 VL |
| | QSGVPARFSGSGSGTDFTLNIHPVEEEDVATYYCHHNRDLPFTFGSGTKLEIK | |
| 265 | DIVVTQSPASLAVSLGQRATISCRASKSVSTSGYSYMHWYQQKPGQPPKLLIYLASNL | Y0188-8 VL |
| | ESGVPARFSGSGSGTDFTLNIHPVEEEDVAIYYCQHSRELPLTFGAGTKLELK | |
| 266 | DIVLTQSPASLAVSLGQRATISCRASKSVSASGYSYMHWYQQKPGQPPKLLIYLASNL | Y0188-9 VL |
| | QSGVPARFSGSGSGTDFTLNIHPVEEEDAATYYCQHSRELPPTFGGGTKLEIK | |
| 267 | DIVLTQSPASLAVFLGQRATISCRASKSVSTSGYSYMHWYQQKAGQPPKLLIYLASNL | Y0188-10 VL |
| | ESGVPARFSGSGSGTDFTLNIHPVEEEDAATYYCHHSRELPITFGSGTKLEMK | |
| 268 | DIVMTQSHKFMSTSVGDRVSITCKASQDVSTAVAWYQEKPGQSPKLLIYWASTRHT | Y0188-14 VL |
| | GVPDRFTGSGSGTDYTLTISSVQAEDLALYYCQQHYSTPLTFGAGTKLELK | |
| 269 | EIVLTQSPGTLSLSPGERATLSCKASQDVSTAVAWYQQKPGQAPRLLIYWASTRHTGI | Y01-14 VL |
| | PDRFSGSGSGTDFTLTISRLEPEDFAVYYCQQHYSTPLTFGQGTKVEIK | |
| 270 | DIVMTQSPLSLPVTPGEPASISCKASQDVSTAVAWYLQKSGQSPQLLIYWASTRHTG | 164-14 VL |
| | VPDRFSGSGSGTDFTLKISRVEAEDVGFYYCQQHYSTPLTFGQGTKLEIK | |
| 271 | DIVMTQSPDSLAVSLGERATINCKASQDVSTAVAWYQQKPGQPPKLLIYWASTRHT | KV4-14 VL |
| | GVPDRFSGSGSGTDFTLTISSLQAEDVAVYYCQQHYSTPLTFGGGTKVEIK | |
| 272 | DIQMTQSPSSLSASVGDRVTITCKASQDVSTAVAWYQQKPGKVPKLLIYWASTRHT | KV1-27-14 VL |
| | GVPSRFSGSGSGTDFTLTISSLQPEDVATYYCQQHYSTPLTFGGGTKVEIK | |
| 273 | DIQLTQSPSFLSASVGDRVTITCKASQDVSTAVAWYQQKPGKAPKLLIYWASTRHTG | KV1-9-14 VL |
| | VPSRFSGSGSGTEFTLTISSLQPEDFATYYCQQHYSTPLTFGGGTKVEIK | |
| 274 | DIQMTQSPSSLSASVGDRVTITCKASQDVSTAVAWYQQKPGKAPKLLLYWASTRHT | KV1-NL1-14 VL |
| | GVPSRFSGSGSGTDYTLTISSLQPEDFATYYCQQHYSTPLTFGGGTKVEIK | |
| 275 | AIRMTQSPFSLSASVGDRVTITCKASQDVSTAVAWYQQKPAKAPKLFIYWASTRHTG | KV1D-43-14 VL |
| | VPSRFSGSGSGTDYTLTISSLQPEDFATYYCQQHYSTPLTFGGGTKVEIK | |
| 276 | EVQLQQSGPELVKPGASVRMSCKASGYTFTDYNIHWVKQSHGKSLEWIGYIYPNNG | HCVR of REGN1103 |
| | DNGYNQKFRGKATLTVDKSSSTAYMELRSLTSDDSAVYYCARGRLRYFDVWGTGTT | (anti-mIL-4Rα) |
| | VTVSS | |
| 277 | NIVLTQSPASLAVSLGQRATISCRASESVDNYGHSFMHWYQQKPGQPPKLLIYLASN | LCVR of REGN1103 (anti- |
| | LESGVPARFSGSGSRTDFTLTLDPVEADDAATYYCQQYNEDPPTFGSGTKLEIK | mIL-4Rα) |

### EXAMPLES

The following examples are put forth so as to provide those of ordinary skill in the art with a complete disclosure and description of how to make and use the methods and compositions of the disclosure and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Effects of IL-4R Inhibition in a Mouse Model of Eosinophilic Gastritis

An in vivo experiment was performed to evaluate the effects of prophylactic blockade of IL-4Rα in a mouse model of eosinophilic gastritis generated by overexpression of mouse interleukin-25 (mIL-25). Overexpression of mIL-25 induces eosinophilia and type-2-driven pathologies in stomach tissue. Therefore, mIL-25 overexpression was used to generate a mouse model of eosinophilia, including eosinophilic gastritis.

Overexpression of mIL-25 was induced using hydrodynamic delivery (HDD) of 25 μg plasmid DNA. Mice were administered mIL-25 or vector control via HDD on day 0. Blockade of IL-4Rα in this mouse model was mediated by using an antibody to mouse IL-4Rα, REGN1103, that was administered via subcutaneous (SC) injection on days −4, 1, and 3; REGN1103 is a monoclonal antibody specific for mouse IL-4Rα that has binding characteristics and functional properties similar to those of dupilumab in humans. REGN1103 has the HCVR sequence of SEQ ID NO:276 and the LCVR sequence of SEQ ID NO:277. The mice were sacrificed on day 8 (i.e., 8 days after induction of mIL-25 overexpression and 5 days after the last antibody dose) and pathologies associated with mIL-25 overexpression in stomach tissue were assayed by flow cytometry, determination of mRNA levels of eotaxins, and histology.

Flow cytometry: To enable flow cytometric analysis of circulating versus tissue-infiltrating immune cells in the lung, 5 minutes prior to sacrifice, mice were injected IV with BV650 anti-CD45 to selectively label immune cells still in the vasculature while leaving cells that had infiltrated the stomach tissue unlabeled. The dissociated stomach tissue was then subsequently stained with a mix of antibodies that included an anti-CD45 antibody of the same clone that was injected prior to sacrifice but conjugated to a different fluorophore (BV786 anti-CD45). This ex vivo CD45 staining preferentially labels cells that were in the stomach tissue and were protected from the injected, in vivo, CD45 antibody.

A longitudinal section of stomach tissue was isolated and placed in ice-cold enzymatic digest solution (0.6% FBS, 0.5 mg/ml CollagenaseD, 0.1 mg/ml DNase I, 1 mg/ml Dispase II in HBSS without calcium and magnesium). The stomach tissue was minced with scissors into fine pieces, transferred to a conical tube with additional enzymatic digest solution, and incubated with shaking at 37° C. for 45 minutes. After incubation, samples were dissociated, filtered through a 70 μm mesh, collected, and pelleted by centrifugation at 4° C. The supernatant was removed, and sample cells were resuspended in Dulbecco's phosphate-buffered saline (DPBS) for staining.

Cells were stained with Live/Dead Fixable Dead Cell Stain in DPBS to allow exclusion of dead cells. Cells were incubated with anti-mouse CD16/CD32 (Fc shield) followed by incubation with APC anti-CD11c, BV421 anti-Siglec-F, BV786 anti-CD45, PE anti-CD200R3, and APC-eFluor CD117 monoclonal antibody cocktail. Samples were acquired on a LSR Fortessa X 20 or a FACSymphony cell analyzer using the HTS attachment. Eosinophils were defined as intact cells, singlets, live (low LIVE/DEAD viability dye signal), CD45+ (BV786), CDC11^{lo-int}, SiglecF^{hi}. Data analysis was performed using FlowJo v10 software.

Real-time PCR analysis: Tissues were homogenized, and chloroform was used for phase separation. The aqueous phase, containing total RNA, was purified using MagMAX™-96 for Microarrays Total RNA Isolation Kit according to manufacturer's specifications. Genomic DNA was removed using RNase-Free DNase Set. Beta-actin was used as a housekeeping gene. Gene expression was normalized to Actb (Beta-actin).

Histology and pathology scoring: Sections of stomach tissue were collected at necroscopy and processed for hematoxylin and eosin staining. Following staining, whole slide images of tissue sections were blindly evaluated by a board-certified veterinary pathologist. A semi-quantitative grading system (0—no lesions, 1—minimal lesions, 2—mild lesions, 3-moderate lesions, 4—severe lesions) was employed to score the following histological features in the stomach: orthokeratotic hyperkeratosis, squamous epithelial hyperplasia, vacuolation of squamous epithelium, and cytoplasmic inclusions in squamous epithelium in non-glandular stomach, hypertrophy/hyperplasia of mucous cell in the glandular stomach, mixed cell inflammation (mononuclear cells, neutrophils, and eosinophils), edema, and hyperplasia/hypertrophy of the tunica muscularis. Total pathology score was determined by summing the scores of individual histological features, with a maximum score of 36.

Statistical analyses: All data are presented as group mean±standard deviation (SD). For the experiment showing eosinophils as a frequency of CD45+ cells, normality was tested using Shapiro-Wilk test, and p values were determined using Brown-Forsythe and Welch analysis of variance (ANOVA) tests and multiple comparisons performed using Dunnett's T3 multiple comparisons test. For the experiment showing levels of Ccl11 and Ccl24 mRNA in stomach tissue, normality was tested using Shapiro-Wilk test, and p-values were determined using Kruskal-Wallis test and multiple comparisons done using Dunn's multiple comparisons test. For pathology scoring, statistical analysis of variance of the data was performed by one way ANOVA followed Tukey's Honest Significance Difference (HSD) test. P-values less than 0.5 were considered statistically significant, and statistical analyses were performed with GraphPad Prism software.

### Results

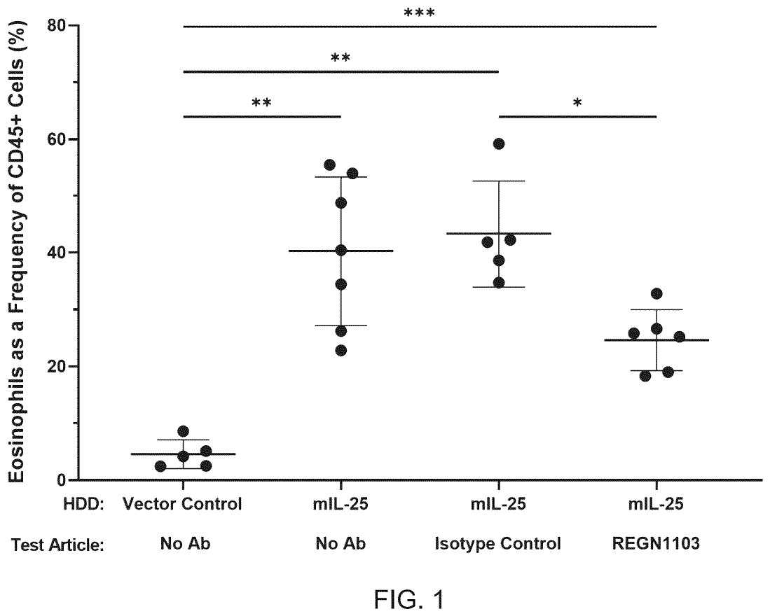

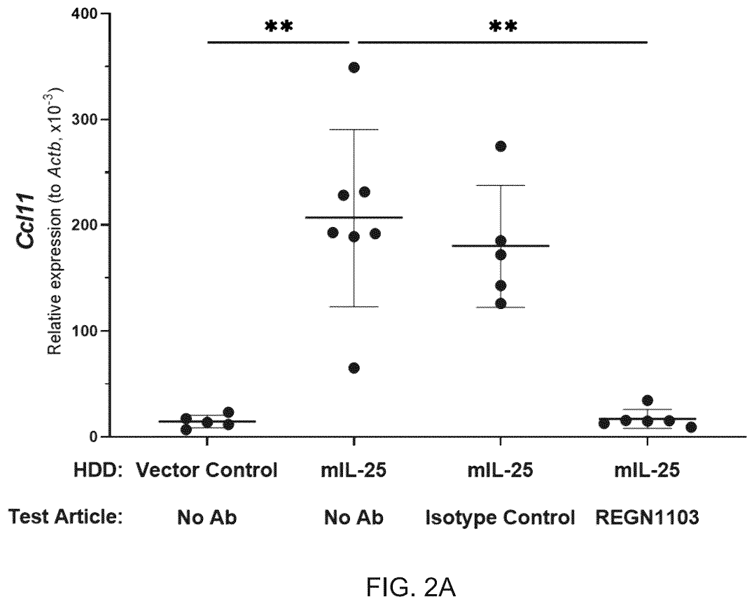

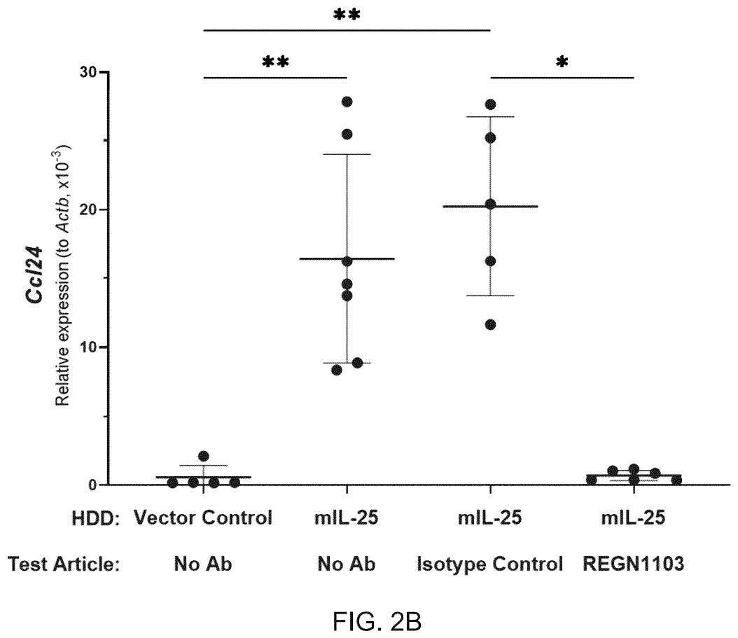

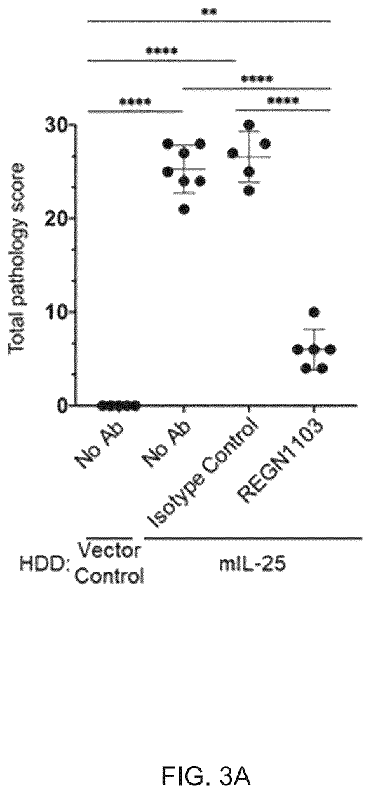

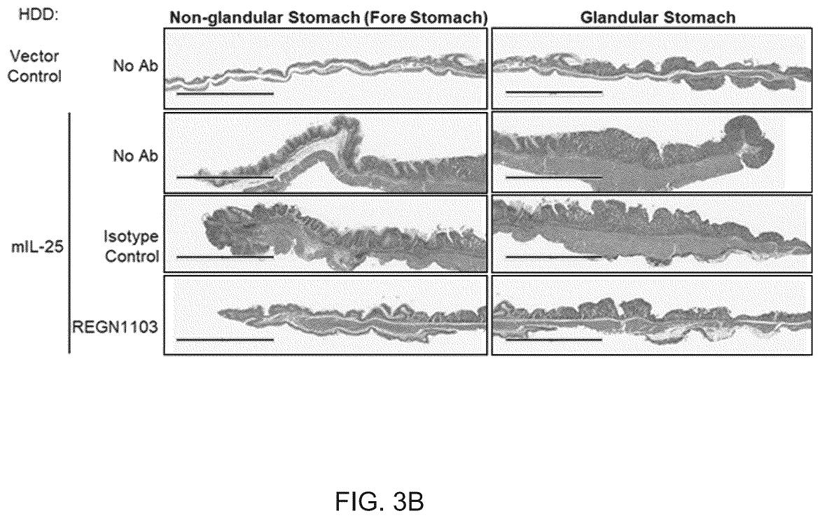

The effect of REGN1103 on the frequency of eosinophils as a percentage of CD45+ cells, found in stomach tissue of a mouse model of eosinophilic gastritis was measured. Overexpression of mouse IL-25 significantly increased the frequency of eosinophils, and, among mice overexpressing IL-25, administration of 25 mg/kg REGN1103 significantly reduced the frequency of eosinophils found in stomach tissue compared with administration of isotype control antibody (FIG. 1). Additionally, among mice overexpressing IL-25, administration of REGN1103 significantly reduced the expression of Ccl11 and Ccl24 mRNA in stomach tissue compared to mice administered isotype control antibody or no Ab control, and REGN1103 reduced Ccl11 and Ccl24 mRNA in stomach tissues of mice overexpressing mIL-25 to levels indistinguishable from mice administered vector control via HDD (FIGS. 2A and 2B). Stained sections of stomach tissue were evaluated to determine the degree of pathology. Mice overexpressing IL-25 and administered either isotype control antibody or no Ab control exhibited significantly higher average pathology scores compared with mice overexpressing IL-25 that were administered REGN1103 (FIGS. 3A and 3B). Mice without exogenous IL-25 expression and administered no Ab control showed no pathology.

In summary, in the stomach tissue of a mouse model of eosinophilic gastritis, administration of 25 mg/kg REGN1103 via SC injection on days −4, 1, and 3 induced a statistically significant reduction in the frequency of eosinophils, Ccl11 and Ccl24 mRNA levels, and the development of lesions.

### Example 2: Clinical Study to Investigate the Efficacy and Safety of Dupilumab in Adult and Adolescent Patients with Eosinophilic Gastritis with or without Eosinophilic Duodenitis

This example describes a Phase 2/3, randomized, 3-part study to investigate the efficacy and safety of dupilumab in adult and adolescent participants with eosinophilic gastritis (EoG) with or without eosinophilic duodenitis (EoD). Part A (phase 2) and Part B (phase 3) are 24-week, randomized, double-blind, placebo-controlled study periods and Part C is a 28-week, extended active treatment period that will enroll participants from Part A and Part B (i.e., overall exposure of approximately 52 weeks).

For Part A, the primary objective is to determine the treatment effect of dupilumab treatment compared with placebo in adult and adolescent participants with EoG with or without EoD after 24 weeks of treatment as assessed by histological measures and to inform/confirm the final sample size determination for Part B.

For Part B, the primary objective is to demonstrate the efficacy of dupilumab treatment compared with placebo in adult and adolescent participants with EoG (with or without EoD) after 24 weeks of treatment as assessed by histological and clinical measures.

For Part C, the primary objective is to assess the safety and efficacy of dupilumab treatment in adult and adolescent participants with EoG (with or without EoD) after up to 52 weeks of treatment as assessed by histological and clinical measures.

The secondary objectives of the study are:
To determine the treatment effect of dupilumab treatment compared with placebo in adult and adolescent participants with EoG (with or without EoD) after 24 weeks of treatment as assessed by clinical measures (Part A only).
To evaluate the safety, tolerability, and immunogenicity of dupilumab treatment for up to 52 weeks in adult and adolescent participants with EoG (with or without EoD)
To characterize the trough concentrations of functional dupilumab over time following administration of dupilumab in adult and adolescent participants with EoG (with or without EoD)
To evaluate the effects of dupilumab on transcriptomic signatures associated with EoG (with or without EoD) and type 2 inflammation.

Overall, approximately 279 participants will be enrolled in the study.

In Part A, approximately 54 adult and adolescent participants will be randomized in a 1:1 ratio to either dupilumab 300 mg once weekly (QW) or matching placebo administered subcutaneously (SC). At the end of the double-blind treatment visit (week 24) in Part A, eligible participants may enter a 28-week extended active treatment period (Part C). All participants from Part A will receive active treatment with dupilumab 300 mg QW during Part C. During Part C, participants from Part A will remain blinded to their treatment assignment in Part A.

In Part B, approximately 225 adult and adolescent participants will be randomized in a 1:1:1 ratio to either dupilumab 300 mg QW, dupilumab 300 mg every 2 weeks (Q2W), or matching placebo administered SC. A sample size re-estimation for Part B will be assessed, which may result in an adjustment to the Part B sample size. At the end of the double-blind treatment visit (week 24) in Part B, eligible participants may enter a 28-week extended active treatment period (Part C). During Part C, participants from Part B will remain blinded to their treatment assignment in Part B and treatment regimen in Part C.

Participants from Part B who are randomized to placebo during the double-blind treatment period will be re-randomized in a 1:1 ratio to dupilumab 300 mg QW or dupilumab 300 mg Q2W during Part C. All other participants will remain on the same dupilumab dose regimen to which they are randomized during the double-blind treatment period. Participants randomized to dupilumab 300 mg Q2W (in Part B or Part C) will also receive matching placebo administered Q2W in Part C alternating with dupilumab doses so the injection frequency will match the other group for regimen-blinding purposes.

Inclusion Criteria: The key inclusion criteria for Parts A and B include:
Male or female 12 years of age. (Note: Adolescent participants will only be enrolled at study sites in countries/regions as permitted by local regulatory authorities and ECs.)
Documented endoscopic biopsy supporting a pathologic diagnosis of EoG at least 3 months prior to screening. (Note: There should be sufficient documentation to support a pathologic diagnosis of EoG.)
Screening endoscopic biopsies (which will serve as baseline) with a demonstration of eosinophilic infiltration for a diagnosis of EoG (≥30 eos/hpf in at least 5 distinct hpfs in the stomach) as determined by central histology assessment of biopsies collected during the screening endoscopy without any other significant cause for the gastric eosinophilia (e.g., parasitic infections, malignancy).
Completed at least 11 of 14 days of EoG/EoD-SQ eDiary data entry in the 2 weeks prior to the baseline visit
History (by participant report) of at least 2 episodes of EoG symptoms (e.g., stomach pain, stomach cramping, nausea, bloating, early satiety, loss of appetite) per week in the 8 weeks prior to screening
An average TSS of ≥20 calculated using data collected via the EoG/EoD-SQ eDiary per week for the 2 weeks prior to baseline. An average severity score of 4 (on a scale of 0-10) per week for the 2 weeks prior to baseline for at least 2 of the following 6 symptoms: stomach cramping, stomach pain, nausea, bloating, early satiety, loss of appetite.

Exclusion criteria: The key exclusion criteria for Parts A and B include:
Body weight<40 kg
Prior participation in a dupilumab clinical trial, or past or current treatment with dupilumab
Helicobacter pylori infection
Any esophageal stricture unable to be passed with a standard, diagnostic, upper endoscope or any critical esophageal stricture that requires dilation at screening
History of achalasia, Crohn's disease, eosinophilic colitis, ulcerative colitis, celiac disease, and prior gastric or duodenal surgery
Other causes of gastric and, if applicable, duodenal eosinophilia or the following conditions: eosinophilic granulomatosis with polyangiitis (Churg-Strauss syndrome) or hyper-eosinophilic syndrome
History of bleeding disorders, esophageal or gastric varices that, in the opinion of the investigator, would put the participant at undue risk for significant complications from an endoscopy procedure
Initiation or change of a food-elimination diet regimen or re-introduction of a previously eliminated food group in the 4 weeks prior to screening. Participants on a food-elimination diet must remain on the same diet throughout the study.
Receiving enteral (tube feeding) or parenteral nutrition at screening
Treatment with swallowed topical corticosteroids not for the treatment of EoG (with or without EoD) and/or EoE within 8 weeks prior to baseline

Study Treatment: The dosing regimens of the study are as follows:

Part A: 24-week double-blind treatment period
Dupilumab 300 mg administered QW SC
Placebo administered QW SC containing the same formulation as dupilumab without active substance

Part B: 24-week double-blind treatment period
Dupilumab 300 mg administered QW SC
Dupilumab 300 mg administered Q2W SC
   For participants randomized to dupilumab 300 mg Q2W, there will be an SC injection of matching placebo alternating with dupilumab doses so the injection frequency will be identical (QW) for both groups for regimen-blinding purposes.
Placebo administered QW SC containing the same formulation as dupilumab without active substance

Part C: 28-week extended active treatment period
All participants from Part A will receive dupilumab 300 mg QW SC during Part C.
All participants from Part B will receive either dupilumab 300 mg QW or Q2W SC during Part C.
   For participants randomized to dupilumab 300 mg Q2W, there will be an SC injection of matching placebo alternating with dupilumab doses so the injection frequency will be identical (QW) for both groups for regimen-blinding purposes.

Randomization for Part A will also be stratified by use of systemic corticosteroids or STC (with or without EoD) at randomization (yes vs. no) and organ involvement at baseline (stomach only vs. stomach and duodenum). Randomization for Part B will be stratified by region (Japan vs. rest of world), age (≥18 vs. ≥12 to <18 years of age), use of systemic corticosteroids or STCs for EoG (with or without EoD) at randomization (yes vs. no), and organ involvement at baseline (stomach only vs. stomach and duodenum). Participants from study sites in Japan will not be further stratified by the other 3 stratification factors (age, use of systemic or STCs, organ involvement at baseline).

### Primary and Secondary Endpoints:

The primary endpoint for Part A of the study is the proportion of participants achieving a peak gastric eosinophil count of 56 eos/hpf at week 24 (i.e., histological responder).

The two primary endpoints for Part B of the study are: proportion of participants achieving a peak gastric eosinophil count of 56 eos/hpf at week 24 (i.e., histological responder), and absolute change in the EoG/EoD-SQ TSS from baseline to week 24.

The secondary efficacy endpoints for Part A, Part B, and Part C of the study are:
proportion of participants achieving both a peak gastric eosinophil count of ≤6 eos/hpf and a peak duodenal eosinophil count of ≤15 eos/hpf at week 24 (assessed for those with both gastric and duodenal involvement)
proportion of participants achieving a peak duodenal eosinophil count of ≤15 eos/hpf at week 24 (assessed for those with duodenal involvement)
absolute change in the EoG/EoD-SQ TSS from baseline to week 24 (Part A only [second primary endpoint for Part B])
percent change in the EoG/EoD-SQ TSS from baseline to week 24
percent change in peak gastric tissue eosinophil count (eos/hpf) from baseline to week 24
proportion of participants achieving a peak gastric tissue eosinophil count of <30 eos/hpf at week 24
percent change in peak duodenal tissue eosinophil count (eos/hpf) from baseline to week 24 (assessed for only those with duodenal involvement)
proportion of participants achieving a peak duodenal tissue eosinophil count of <30 eos/hpf at week 24 (assessed for only those with duodenal involvement)
absolute change in EoG scores from the EoG Histology Scoring System (EoGHSS) from baseline to week 24
change from baseline in frequency of diarrhea episodes at week 24 (assessed for only those with diarrhea at baseline)
change from baseline in frequency of vomiting episodes at week 24 (assessed for only those with vomiting at baseline)
change from baseline to week 24 in the normalized Enrichment Scores (NES) for the type 2 inflammation transcriptome signature (assessed on gastric tissue)
change from baseline to week 24 in the NES for the type 2 inflammation transcriptome signature (assessed on duodenal tissue from participants with EoD)
change from baseline to week 24 in the NES for the EoG disease signature (EGDP) transcriptome signature (assessed on gastric tissue); and
proportion of participants who receive rescue medications or procedures during the 24-week placebo-controlled treatment period.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method of treating, preventing, or ameliorating at least one symptom of eosinophilic gastroenteritis, the method comprising administering to a subject having eosinophilic gastroenteritis one or more doses of an interleukin-4 receptor (IL-4R) antagonist.

2. The method of claim 1, wherein the subject has eosinophilic gastritis (EoG) with eosinophilic duodenitis (EoD).

3. The method of claim 1, wherein the subject has eosinophilic gastritis (EoG) without eosinophilic duodenitis (EoD).

4. The method of claim 1, wherein the subject has eosinophilic duodenitis (EoD) without eosinophilic gastritis (EoG).

5. The method of claim 1, wherein the subject has been previously treated with a systemic corticosteroid or a swallowed topical corticosteroid.

6. The method of claim 1, wherein the subject is unresponsive, inadequately responsive, or intolerant to treatment with a systemic corticosteroid or a swallowed topical corticosteroid, or wherein standard of care treatment is contraindicated.

7. A method of reducing the use of a systemic corticosteroid or a swallowed topical corticosteroid in a subject having eosinophilic gastroenteritis, the method comprising administering to the subject one or more doses of an interleukin-4 receptor (IL-4R) antagonist.

8. The method of claim 7, wherein the subject has eosinophilic gastritis (EoG) with eosinophilic duodenitis (EoD).

9. The method of claim 7, wherein the subject has eosinophilic gastritis (EoG) without eosinophilic duodenitis (EoD).

10. The method of claim 7, wherein the subject has eosinophilic duodenitis (EoD) without eosinophilic gastritis (EoG).

11. The method of claim 1, wherein at the start of treatment with the IL-4R antagonist, the subject is on a stable dose of a maintenance systemic corticosteroid or a swallowed topical corticosteroid.

12. The method of claim 1, wherein the subject is ≥12 years old.

13. The method of claim 1, wherein the subject is an adult.

14. The method of claim 1, wherein the subject has a concomitant atopic disease.

15. The method of claim 14, wherein the concomitant atopic disease is a food allergy, atopic dermatitis, asthma, chronic rhinosinusitis, allergic rhinitis, or allergic conjunctivitis.

16. The method of claim 1, wherein prior to the onset of treatment with the IL-4R antagonist the subject:
(i) has an eosinophil count≥30 eos/hpf as measured by endoscopic biopsy in at least five distinct regions of the stomach;
(ii) has an eosinophil count≥30 eos/hpf as measured by endoscopic biopsy in at least three distinct regions of the small intestine;
(iii) has a baseline total symptom score (TSS) of ≥20 as measured by the EoG/EoD Symptom Questionnaire (EoG/EoD-SQ);
(iv) has a baseline average severity score of ≥4 per week for at least two weeks for at least two components of the EoG/EoD-SQ, wherein the components are selected from the group consisting of stomach pain, stomach cramping, nausea, bloating, early satiety, and loss of appetite; and/or
(v) has a history of at least two episodes of EoG symptoms per week for at least 8 weeks, wherein the symptoms are selected from the group consisting of stomach pain, stomach cramping, nausea, bloating, early satiety, and loss of appetite.

17. The method of claim 1, wherein the IL-4R antagonist is an anti-IL-4R antibody or an antigen-binding fragment thereof.

18. The method of claim 1, wherein the IL-4R antagonist is an anti-IL-4R antibody or an antigen-binding fragment thereof that comprises three heavy chain complementarity determining regions (HCDR1, HCDR2 and HCDR3) and three light chain complementarity determining regions (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence of LGS, and the LCDR3 comprises the amino acid sequence of SEQ ID NO:8.

19. The method of claim 1, wherein the IL-4R antagonist is an anti-IL-4R antibody or an antigen-binding fragment thereof that comprises a heavy chain variable region (HCVR) comprising the amino acid sequence of SEQ ID NO:1 and comprises a light chain variable region (LCVR) comprising the amino acid sequence of SEQ ID NO:2.

20. The method of claim 1, wherein the IL-4R antagonist is an anti-IL-4R antibody that comprises a heavy chain comprising the amino acid sequence of SEQ ID NO:9 and a light chain comprising the amino acid sequence of SEQ ID NO:10.

21. The method of claim 1, wherein the IL-4R antagonist is dupilumab.

22. The method of claim 1, wherein the IL-4R antagonist is administered at a dose of about 50 mg to about 600 mg.

23. The method of claim 1, wherein the IL-4R antagonist is administered once a week, once every two weeks, once every three weeks, or once every four weeks.

24. The method of claim 22, wherein the IL-4R antagonist is administered at a dose of about 300 mg QW.

25. The method of claim 22, wherein the IL-4R antagonist is administered at a dose of about 300 mg Q2W.

26. The method of claim 1, wherein the IL-4R antagonist is administered subcutaneously.

27. The method of claim 1, wherein the IL-4R antagonist is administered in combination with a second therapeutic agent or therapy.

28. A method of treating eosinophilic gastroenteritis in a subject, the method comprising administering to the subject a combination therapy comprising (i) an interleukin-4 receptor (IL-4R) antagonist, and (ii) a systemic corticosteroid or a swallowed topical corticosteroid.

29. The method of claim 28, wherein the combination therapy comprises an IL-4R antagonist and a systemic corticosteroid.

30. The method of claim 29, wherein the systemic corticosteroid is prednisone or prednisolone.

31. The method of claim 28, wherein the combination therapy comprises an IL-4R antagonist and a swallowed topical corticosteroid.

32. The method of claim 31, wherein the swallowed topical corticosteroid is budesonide or fluticasone.

33. The method of claim 28, wherein the subject has eosinophilic gastritis (EoG) with eosinophilic duodenitis (EoD).

34. The method of claim 28, wherein the subject has eosinophilic gastritis (EoG) without eosinophilic duodenitis (EoD).

35. The method of claim 28, wherein the subject has eosinophilic duodenitis (EoD) without eosinophilic gastritis (EoG).

36. The method of claim 28, wherein the subject is ≥12 years old.

37. The method of claim 28, wherein the subject is an adult.

38. The method of claim 28, wherein the subject has a concomitant atopic disease.

39. The method of claim 38, wherein the concomitant atopic disease is a food allergy, atopic dermatitis, asthma, chronic rhinosinusitis, allergic rhinitis, or allergic conjunctivitis.

40. The method of claim 28, wherein the subject to be treated with the combination therapy:
(i) has an eosinophil count≥30 eos/hpf as measured by endoscopic biopsy in at least five distinct regions of the stomach;
(ii) has an eosinophil count≥30 eos/hpf as measured by endoscopic biopsy in at least three distinct regions of the small intestine;
(iii) has a baseline total symptom score (TSS) of ≥20 as measured by the EoG/EoD Symptom Questionnaire (EoG/EoD-SQ);
(iv) has a baseline average severity score of ≥4 per week for at least two weeks for at least two components of the EoG/EoD-SQ, wherein the components are selected from the group consisting of stomach pain, stomach cramping, nausea, bloating, early satiety, and loss of appetite; and/or
(v) has a history of at least two episodes of EoG symptoms per week for at least 8 weeks, wherein the symptoms are selected from the group consisting of stomach pain, stomach cramping, nausea, bloating, early satiety, and loss of appetite.

41. The method of claim 28, wherein the IL-4R antagonist is an anti-IL-4R antibody or an antigen-binding fragment thereof.

42. The method of claim 28, wherein the IL-4R antagonist is an anti-IL-4R antibody or an antigen-binding fragment thereof that comprises three heavy chain complementarity determining regions (HCDR1, HCDR2 and HCDR3) and three light chain complementarity determining regions (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence of LGS, and the LCDR3 comprises the amino acid sequence of SEQ ID NO:8.

43. The method of claim 28, wherein the IL-4R antagonist is an anti-IL-4R antibody or an antigen-binding fragment thereof that comprises a heavy chain variable region (HCVR) comprising the amino acid sequence of SEQ ID NO:1 and comprises a light chain variable region (LCVR) comprising the amino acid sequence of SEQ ID NO:2.

44. The method of claim 28, wherein the IL-4R antagonist is an anti-IL-4R antibody that comprises a heavy chain comprising the amino acid sequence of SEQ ID NO:9 and a light chain comprising the amino acid sequence of SEQ ID NO:10.

45. The method of claim 28, wherein the IL-4R antagonist is dupilumab.

46. The method of claim 28, wherein the IL-4R antagonist is administered at a dose of about 50 mg to about 600 mg.

47. The method of claim 28, wherein the IL-4R antagonist is administered once a week, once every two weeks, once every three weeks, or once every four weeks.

48. The method of claim 46, wherein the IL-4R antagonist is administered at a dose of about 300 mg QW.

49. The method of claim 4, wherein the IL-4R antagonist is administered at a dose of about 300 mg Q2W.

50. The method of claim 28, wherein the IL-4R antagonist is administered subcutaneously.

51. The method of claim 1, wherein the IL-4R antagonist is contained in a container selected from the group consisting of a glass vial, a syringe, a pre-filled syringe, a pen delivery device, and an autoinjector.

52. The method of claim 51, wherein the IL-4R antagonist is contained in a pre-filled syringe.

53. The method of claim 52, wherein the pre-filled syringe is a single-dose pre-filled syringe.

54. The method of claim 51, wherein the IL-4R antagonist is contained in an autoinjector.

55. The method of claim 51, wherein the IL-4R antagonist is contained in a pen delivery device.
